(19) United States
(12) Patent Application Publication
(10) Pub. No.: US 2006/0063782 A1
(43) Pub. Date: Mar. 23, 2006

(54) 3-Hetero arylmethoxy ! pyridines and their analogues as p38 map kinase inhibitors

(76) Inventors: Christopher William Murray, Cambridge (GB); Michael John Hartshorn, Cambridge (GB); Martyn Frederickson, Cambridge (GB); Miles Stuart Congreve, Cambridge (GB); Alessandro Padova, Roma (IT); Steven John Woodhead, Cambridge (GB); Adrian Liam Gill, Cambridge (GB); Andrew James Woodhead, Cambridge (GB)

Correspondence Address: NIXON & VANDERHYE, PC, 901 NORTH GLEBE ROAD, 11TH FLOOR, ARLINGTON, VA, 22203, US

(21) Appl. No.: 10/519,922

(22) PCT Filed: Jul. 3, 2003
(86) PCT No.: PCT/GB03/02864

§ 371 Date: Jan. 3, 2005

Related U.S. Application Data
(60) Provisional application No. 60/393,121, filed on Jul. 3, 2002.

(30) Foreign Application Priority Data
Jul. 3, 2002 (GB) 0215383.1
Nov. 8, 2002 (GB) 0226149.3

(51) Int. Cl. A61K 31/4965; C07D 213/63; A61K 31/44

(52) U.S. Cl. 514/255.06; 514/345; 544/406; 546/291

(57) ABSTRACT

Compounds of the formula (I), wherein: —X═Y— is selected from —CR<2>=CR<3>— and —CR<2>═N—; R<1> is selected from H, halo, NRR′, NHC(═O)R, NHC(═O)NRR′, NH2SO2R, and C(═O)NRR′; R<2> and R<3> (where present) are independently selected from H, optionally substituted C1-7 alkyl, optionally substituted C5-20 aryl, optionally substituted C3-20 heterocyclyl, halo, amino, amido, hydroxy, ether, thio, thioether, acylamido, ureido and sulfonamino; R<4> is an optionally substituted C5-20 aryl or C5-20 heteroaryl group; and R<5> is selected from R<5′>, halo, NHR<5′>, C(═O)NHR<5′>, OR<5′>, SR<5′>, NHC(═O)R<5′>, NHC(═O)NHR<5′>, NHS(═O)R<5′>, wherein R<5′> is H or C1-3 alkyl (optionally substituted by halo, NH2, OH, SH) are disclosed for use in therapy and for treating diseases ameliorated by inhibiting p38 MAP kinase.

### RELATED APPLICATIONS

This application claims priority to U.S. Provisional Application No. 60/393,121 filed 3 Jul. 2002, United Kingdom Application Number 0215383.1 filed 3 Jul. 2002 and United Kingdom Application Number 0226149.3 filed 8 Nov. 2002, the contents of which are incorporated herein by reference in their entirety.

### TECHNICAL FIELD

This invention relates to pyridine and pyrazine derivatives which inhibit the activity of p38 MAP kinase, and the use of these compounds as pharmaceuticals.

### BACKGROUND

Mitogen-activated protein (MAP) kinases are proline-directed kinases that mediate the effects of numerous extracellular stimuli on a wide array of biological processes, such as cell proliferation, differentiation and death. Three groups of mammalian MAP kinases have been studied in detail: the extracellular signal-regulated kinases (ERK), the c-Jun NH₂— terminal kinases (JNK) and the p38 MAP kinases.

There are five known human isoforms of p38 MAP kinase, p38α, p38β, p38β2, p38γ and p38δ. The p38 kinases, which are also known as cytokine suppressive anti-inflammatory drug binding proteins (CSBP), stress activated protein kinases (SAPK) and RK, are responsible for phosphorylating and activating transcription factors as well as other kinases, and are themselves activated by physical and chemical stress (e.g. UV, osmotic stress), pro-inflammatory cytokines and bacterial lipopolysaccharide (LPS) (Herlaar, E & Brown, Z., Molecular Medicine Today, 5: 439-447 (1999)). The products of p38 phosphorylation have been shown to mediate the production of inflammatory cytokines, including TNF and IL-1, and cyclooxygenase-2 (COX-2). Each of these cytokines has been implicated in numerous disease states and conditions. IL-1 and TNF are also known to stimulate the production of other proinflammatory cytokines such as IL-6 and IL-8.

Interleukin-1 (IL-1) and Tumor Necrosis Factor (TNF) are biological substances produced by a variety of cells, such as monocytes or macrophages. IL-1 has been demonstrated to mediate a variety of biological activities thought to be important in immunoregulation and other physiological conditions such as inflammation (e.g. Dinarello, et al., Rev. Infect. Disease, 6: 51 (1984)). The myriad of known biological activities of IL-1 include the activation of T helper cells, induction of fever, stimulation of prostaglandin or collagenase production, neutrophil chemotaxis, induction of acute phase proteins and the suppression of plasma iron levels.

There are many disease states in which excessive or unregulated IL-1 production is implicated in exacerbating and/or causing the disease. These include rheumatoid arthritis, osteoarthritis, endotoxemia and/or toxic shock syndrome, other acute or chronic inflammatory disease states such as the inflammatory reaction induced by endotoxin or inflammatory bowel disease; tuberculosis, atherosclerosis, muscle degeneration, cachexia, psoriatic arthritis, Reiter's syndrome, gout, traumatic arthritis, rubella arthritis, and acute synovitis. Evidence also links IL-1 activity to diabetes and pancreatic B cells (Dinarello, J. Clinical Immunology, 5: 287-297 (1985)).

Excessive or unregulated TNF production has been implicated in mediating or exacerbating a number of diseases including rheumatoid arthritis, rheumatoid spondylitis, osteoarthritis, gouty arthritis and other arthritic conditions; sepsis, septic shock, endotoxic shock, gram negative sepsis, toxic shock syndrome, adult respiratory distress syndrome, cerebral malaria, chronic pulmonary inflammatory disease, silicosis, pulmonary sarcoisosis, bone resorption diseases, reperfusion injury , graft vs. host reaction, allograft rejections, fever and myalgias due to infection, such as influenza, cachexia secondary to infection or malignancy, cachexia, secondary to acquired immune deficiency syndrome (AIDS), AIDS, ARC (AIDS related complex), keloid formation, scar tissue formation, Crohn's disease, ulcerative colitis, or pyresis.

Interleukin-8 (IL-8) is a chemotactic factor produced by several cell types including mononuclear cells, fibroblasts, endothelial cells, and keratinocytes. Its production from endothelial cells is induced by IL-1, TNF, or lipopolysachharide (LPS). IL-8 stimulates a number of functions in vitro. It has been shown to have chemoattractant properties for neutrophils, T-lymphocytes, and basophils. In addition it induces histamine release from basophils from both normal and atopic individuals as well as lysozomal enzyme release and respiratory burst from neutrophils. IL-8 has also been shown to increase the surface expression of Mac-1 (CD 11 blCD 18) on neutrophils without de novo protein synthesis, this may contribute to increased adhesion of the neutrophils to vascular endothelial cells. Many diseases are characterized by massive neutrophil infiltration. Conditions associated with an increased in IL-8 production (which is responsible for chemotaxis of neutrophil into the inflammatory site) would benefit by compounds which are suppressive of IL-8 production. Recently Chronic Obstructive Pulmonary Disease (COPD) has been linked to raised levels of IL-8 (Barnes et al., Curr. Opin. Pharmacol., 1: 242-7 (2001)). Other conditions linked to IL-8 include acute respiratory distress syndrome (ARDS), asthma, pulmonary fibrosis and bacterial pneumonia.

IL-1 and TNF affect a wide variety of cells and tissues and these cytokines as well as other leukocyte derived cytokines are important and critical inflammatory mediators of a wide variety of disease states and conditions. The inhibition of these cytokines is of benefit in controlling, reducing and alleviating many of these disease states.

Inhibition of signal transduction via p38, which in addition to IL-1, TNF and IL-8 described above is also required for the synthesis and/or action of several additional pro-inflammatory proteins (i.e., IL-6, GM-CSF, COX-2, collagenase and stromelysin), is expected to be a highly effective mechanism for regulating the excessive and destructive activation of the immune system. This expectation is supported by the potent and diverse anti-inflammatory activities described for p38 kinase inhibitors (Badger, et al., J. Pharm. Exp. Thera., 279: 1453-1461(1996); Griswold, et at., Pharmacol. Comm., 7: 323-229 (1996)).

Activation of immune cells by antigens, cytokines and other regulatory molecules can lead to activation of p38. In disease conditions where for example lymphocyte activation occurs inappropriately to self (auto-immune diseases) or foreign (e.g. allergic diseases) antigens then suppression of the cell response by p38 inhibitors could be beneficial in treating the disease. Other acute and chronic inflammatory diseases resulting from excessive leucocyte activation may also benefit from inhibition of this pathway using raf inhibitors for example contact hypersensitivity, arthritis, eczema, COPD, Alzheimers disease.

A number of inhibitors of p38 MAP kinase have been previously disclosed. Smith-Kline Beecham's SB 203580 (see WO 93/14081) has the structure:

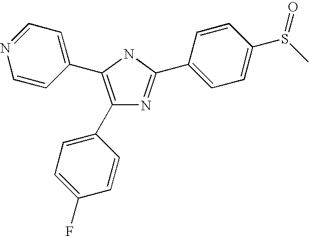

Zeneca have derived (WO 99/15164) compounds having structures related to: 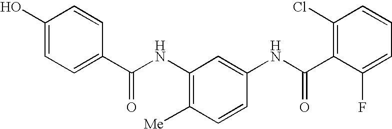
which exhibit inhibition of p38 activity.

Bayer have disclosed a series of compounds which act as p38 MAP kinase inhibitors (WO 99/32111); one such compound has the structure:

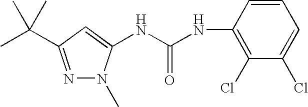

Vertex have developed compounds as p38 MAP kinase inhibitors, with structures such as that shown below (WO 99/00357).

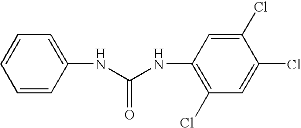

Boehringer Ingelheim have disclosed numerous compounds said to inhibit proinflammatory cytokines, such as TNF and IL-1, in, for example WO 00/43384. An example of a compound disclosed in that patent application is:

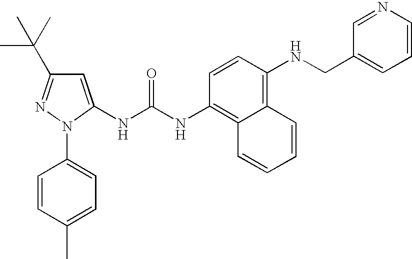

### SUMMARY OF THE INVENTION

The present inventors have discovered that certain pyridine and pyrazine derivatives can be used as pharmaceuticals, and in particular can be used to inhibit the activity of p38 MAP kinase.

Accordingly, the first aspect of the present invention provides a compound of the formula I: 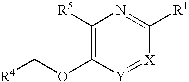
wherein: P —X═Y— is selected from —CR²═CR³— and —CR²═N—;

R¹ is selected from H, halo, NRR′, NHC(═O)R, NHC(═O)NRR′, NH₂SO₂R, and C(═O)NRR′, where R and R′ are independently selected from H and C₁₋₄ alkyl, and are optionally substituted by OH, NH₂, SO₂—NH₂, C₅₋₂₀ carboaryl, C₅₋₂₀ heteroaryl and C₃₋₂₀ heterocyclyl, or may together form, with the nitrogen atom to which they are attached, an optionally substituted nitrogen containing C₅₋₇ heterocyclyl group;

R² and R³ (where present) are independently selected from H, optionally substituted C₁₋₇ alkyl, optionally substituted C₅₋₂₀ aryl, optionally substituted C₃₋₂₀ heterocyclyl, halo, amino, amido, hydroxy, ether, thio, thioether, acylamido, ureido and sulfonamino;

R⁴ an optionally substituted C₅₋₂₀ carboaryl or C₅₋₂₀ heteroaryl group; and

R⁵ is selected from R^{5′}, halo, NHR^{5′}, C(═O)NHR^{5′}, OR^{5′}, SR^{5′}, NHC(═O)R^{5′}, NHC(═O)NHR^{5′}, NHS(═O)₂R^{5′}, wherein R^{5′} is H or C₁₋₃ alkyl (optionally substituted by halo, NH₂, OH, SH);

and pharmaceutically acceptable salts thereof for use in a method of therapy.

The two possibilities for —X═Y— result in compounds of formulae Ia and Ib: 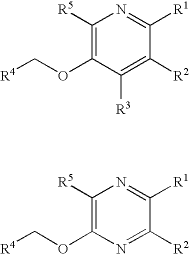
where R¹, R², R³, R⁴ and R⁵ are as defined above.

Particularly preferred compounds of the present invention are of formulae IIa and IIb: 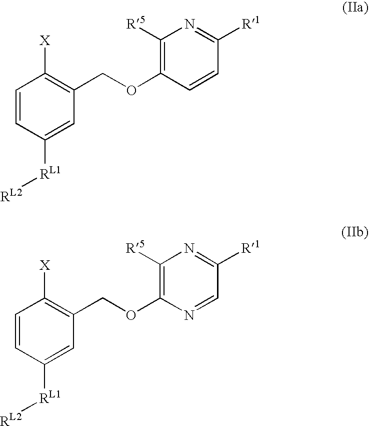
wherein:

R′¹ is selected from H, NR^{C1}R^{C2}, NHC(═O)R^{C1}, NHC(═O)NR^{C1}R^{C2}, NH₂SO₂R^{C1}, and C(═O)NR^{C1}R^{C2}, where R^{C1} and R^{C2} are independently selected from H and C₁₋₄ alkyl, and are optionally substituted by OH, NH₂, C₅₋₂₀ carboaryl, and C₅₋₂₀ heteroaryl, or may together form, with the nitrogen atom to which they are attached, an optionally substituted nitrogen containing C₅₋₇ heterocyclyl group;

R′⁵ is selected from H and NH₂;

X is selected from H and halo;

R^{L1} is selected from —NH—C(═O)—, —NH—C(═O)—NH—, —NH—C(═O)—O— or —O—C(═O)—NH—;

R^{L2} is selected from H, optionally substituted C₅₋₂₀ carboaryl and optionally substituted C₅₋₂₀ heteroaryl, except that R^{L2} cannot be H when R^{L1} is —NH—C(═O)—O—.

A second aspect of the present invention provides a compound of formula IIa or IIb, and isomer, salts, solvates and prodrugs thereof.

A third aspect of the present invention provides a composition comprising a compound of the first aspect and a pharmaceutically acceptable carrier or diluent.

A fourth aspect of the present invention provides the use of a compound of the first aspect of the invention for the manufacture of a medicament for use in the treatment of condition ameliorated by the inhibition of p38 MAP kinase.

Conditions ameliorated by the inhibition of p38 MAP kinase are discussed above, and include, but are not limited to, rheumatoid arthritis, osteoarthritis, rheumatoid spondylitis, gouty arthritis, traumatic arthritis, rubella arthritis, psoriatic arthritis, and other arthritic conditions; Alzheimer's disease; toxic shock syndrome, the inflammatory reaction induced by endotoxin or inflammatory bowel disease; tuberculosis, atherosclerosis, muscle degeneration, Reiter's syndrome, gout, acute synovitis, sepsis, septic shock, endotoxic shock, gram negative sepsis, adult respiratory distress syndrome, cerebral malaria, chronic pulmonary inflammatory disease, silicosis, pulmonary sarcoisosis, bone resorption diseases, reperfusion injury , graft vs. host reaction, allograft rejections, fever and myalgias due to infection, such as influenza, cachexia, in particular cachexia secondary to infection or malignancy, cachexia secondary to acquired immune deficiency syndrome (AIDS), AIDS, ARC (AIDS related complex), keloid formation, scar tissue formation, Crohn's disease, ulcerative colitis, pyresis, chronic obstructive pulmonary disease (COPD), acute respiratory distress syndrome (ARDS), asthma, pulmonary fibrosis and bacterial pneumonia.

Thus, further aspects of the present invention provide the use of a compound of the first aspect of the invention for the manufacture of a medicament for use in the treatment of: arthritic conditions, including rheumatoid arthritis and rheumatoid spondylitis; or inflammatory bowel disease, including Crohn's disease and ulcerative colitis.

Another aspect of the invention provides a compound of the first aspect of the invention for use in a method of treatment of the human or animal body.

Another aspect of the invention provides a method of inhibiting p38 MAP kinase, in vitro or in vivo, comprising contacting a cell with an effective amount of a compound of the first aspect of the invention.

Another aspect of the invention pertains to a method for the treatment of a condition ameliorated by the inhibition of p38 MAP kinase comprising administering to a subject suffering from said a condition ameliorated by the inhibition of p38 MAP kinase a therapeutically-effective amount of a compound of the first aspect of the invention.

Definitions

The phrase “optionally substituted,” as used herein, pertains to a parent group which may be unsubstituted or which may be substituted.

Unless otherwise specified, the term “substituted,” as used herein, pertains to a parent group which bears one or more substituents. The term “substituent” is used herein in the conventional sense and refers to a chemical moiety which is covalently attached to, appended to, or if appropriate, fused to, a parent group. A wide variety of substituents are well known, and methods for their formation and introduction into a variety of parent groups are also well known.

The substituents, and groups listed above, are described in more detail below.

C₁₋₇ alkyl: The term “C₁₋₇ alkyl”, as used herein, pertains to a monovalent moiety obtained by removing a hydrogen atom from a carbon atom of a hydrocarbon compound having from 1 to 7 carbon atoms, which may be aliphatic or alicyclic, and which may be saturated, partially unsaturated, or fully unsaturated. Thus, the term “alkyl” includes the sub-classes alkenyl, alkynyl, cycloalkyl, etc., discussed below.

Examples of saturated alkyl groups include, but are not limited to, methyl (C₁), ethyl (C₂), propyl (C₃), butyl (C₄), pentyl (C₅), hexyl (C₆) and heptyl (C₇)

Examples of saturated linear alkyl groups include, but are not limited to, methyl (C₁), ethyl (C₂), n-propyl (C₃), n-butyl (C₄), n-pentyl (amyl) (C₅), n-hexyl (C₆), and n-heptyl (C₇) .

Examples of saturated branched alkyl groups include iso-propyl (C₃), iso-butyl (C₄), sec-butyl (C₄), tert-butyl (C₄), iso-pentyl (C₅), and neo-pentyl (C₅).

C₃₋₇ Cycloalkyl: The term “C₃₋₇ cycloalkyl” as used herein, pertains to an alkyl group which is also a cyclyl group; that is, a monovalent moiety obtained by removing a hydrogen atom from an alicyclic ring atom of a cyclic hydrocarbon (carbocyclic) compound, which moiety has from 3 to 7 ring atoms. Preferably, each ring has from 3 to 7 ring atoms.

Examples of saturated cylcoalkyl groups include, but are not limited to, those derived from: cyclopropane (C₃), cyclobutane (C₄), cyclopentane (C₅), cyclohexane (C₆) and cycloheptane (C₇) .

C₂₋₇ Alkenyl: The term “C₂₋₇ alkenyl” as used herein, pertains to an alkyl group having one or more carbon-carbon double bonds.

Examples of unsaturated alkenyl groups include, but are not limited to, ethenyl (vinyl, —CH═CH₂), 1-propenyl (—CH═CH—CH₃), 2-propenyl (allyl, —CH—CH═CH₂), isopropenyl (—C(CH₃)═CH₂), butenyl (C₄), pentenyl (C₅), and hexenyl (C₆).

Examples of unsaturated cyclic alkenyl groups, which are also referred to herein as “cycloalkenyl” groups, include, but are not limited to, cyclopropenyl (C₃), cyclobutenyl (C₄), cyclopentenyl (C₅), and cyclohexenyl (C₆).

C₂₋₇ Alkynyl: The term “C₂₋₇ alkynyl”, as used herein, pertains to an alkyl group having one or more carbon-carbon triple bonds.

Examples of unsaturated alkynyl groups include, but are not limited to, ethynyl (ethinyl, —C≡CH) and 2-propynyl (propargyl, —CH₂—C≡CH).

C₁₋₄ alkyl: The term “C₁₋₄ alkyl”, as used herein, pertains to a monovalent moiety obtained by removing a hydrogen atom from a carbon atom of a hydrocarbon compound having from 1 to 4 carbon atoms, which may be aliphatic or alicyclic, and which may be saturated, partially unsaturated, or fully unsaturated. Thus, the term “C₁₋₄ alkyl” includes the sub-classes “C₂₋₄ alkenyl”, “C₂₋₄ alkynyl” and “C₂₋₄ cycloalkyl”. Examples of these moieties are given above.

C₃₋₂₀ Heterocyclyl: The term “C₃₋₂₀ heterocyclyl” as used herein, pertains to a monovalent moiety obtained by removing a hydrogen atom from a ring atom of a heterocyclic compound, which moiety has from 3 to 20 ring atoms, of which from 1 to 10 are ring heteroatoms. Preferably, each ring has from 3 to 7 ring atoms, of which from 1 to 4 are ring heteroatoms, which include N, O and S.

Examples of monocyclic heterocyclyl groups include, but are not limited to, those derived from:

N₁: aziridine (C₃), azetidine (C₄), pyrrolidine (tetrahydropyrrole) (C₅), pyrroline (e.g., 3-pyrroline, 2,5-dihydropyrrole) (C₅), 2H-pyrrole or 3H-pyrrole (isopyrrole, isoazole) (C₅), piperidine (C₆), dihydropyridine (C₆), tetrahydropyridine (C₆), azepine (C₇);

O₁: oxirane (C₃), oxetane (C₄), oxolane (tetrahydrofuran) (C₅), oxole (dihydrofuran) (C₅), oxane (tetrahydropyran) (C₆), dihydropyran (C₆), pyran (C₆), oxepin (C₇);

S₁: thiirane (C₃), thietane (C₄), thiolane (tetrahydrothiophene) (C₅), thiane (tetrahydrothiopyran) (C₆), thiepane (C₇);

O₂: dioxolane (C₅), dioxane (C₆), and dioxepane (C₇);

O₃: trioxane (C₆);

N₂: imidazolidine (C₅), pyrazolidine (diazolidine) (C₅), imidazoline (C₅) pyrazoline (dihydropyrazole) (C₅), piperazine (C₆);

N₁O₁: tetrahydrooxazole (C₅), dihydrooxazole (C₅), tetrahydroisoxazole (C₅), dihydroisoxazole (C₅), morpholine (C₆), tetrahydrooxazine (C₆), dihydrooxazine (C₆), oxazine (C₆);

N₁S₁: thiazoline (C₅), thiazolidine (C₅), thiomorpholine (C₆);

N₂O₁: oxadiazine (C₆);

O₁S₁: oxathiole (C₅) and oxathiane (thioxane) (C₆); and,

N₁O₁S₁: oxathiazine (C₆).

Nitrogen containing C₅₋₇ heterocyclyl: The term “nitrogen containing C₅₋₇ heterocyclyl” as used herein, pertains to a monovalent moiety obtained by removing a hydrogen atom from a ring atom of a heterocyclic compound, which moiety has from 5 to 7 ring atoms, of which a least one is a nitrogen ring atom. Examples of nitrogen containing C₅₋₇ heterocyclyl groups include, but are not limited to, those derived from:

N₁: pyrrolidine (tetrahydropyrrole) (C₅), pyrroline (e.g., 3-pyrroline, 2,5-dihydropyrrole) (C₅), 2H-pyrrole or 3H-pyrrole (isopyrrole, isoazole) (C₅), piperidine (C₆), dihydropyridine (C₆), tetrahydropyridine (C₆), azepine (C₇);

N₂: imidazolidine (C₅), pyrazolidine (diazolidine) (C₅), imidazoline (C₅), pyrazoline (dihydropyrazole) (C₅), piperazine (C₆);

N₁O₁: tetrahydrooxazole (C₅), dihydrooxazole (C₅), tetrahydroisoxazole (C₅), dihydroisoxazole (C₅), morpholine (C₆), tetrahydrooxazine (C₆), dihydrooxazine (C₆), oxazine (C₆);

N₁S₁: thiazoline (C₅), thiazolidine (C₅) thiomorpholine (C₆);

N₂O₁: oxadiazine (C₆);

N₂S₁: thiadiazole (C₅); and,

N₁O₁S₁: oxathiazine (C₆).

C₅₋₂₀ carboaryl: The term “C₅₋₂₀ carboaryl” as used herein, pertains to a monovalent moiety obtained by removing a hydrogen atom from an aromatic ring atom of an aromatic compound, which moiety has from 5 to 20 carbon ring atoms. Preferably, each ring has from 5 to 7 ring atoms.

Examples of carboaryl groups include, but are not limited to, those derived from benzene (i.e. phenyl) (C₆), naphthalene (C₁₀), azulene (C₁₀), anthracene (C₁₄), phenanthrene (C₁₄), naphthacene (C₁₈), and pyrene (C₁₆).

Examples of aryl groups which comprise fused rings, at least one of which is an aromatic ring, include, but are not limited to, groups derived from indene (C₉), isoindene (C₉), and fluorene (C₁₃).

C₅₋₂₀ heteroaryl: The term “C₅₋₂₀ heteroaryl” as used herein, pertains to a monovalent moiety obtained by removing a hydrogen atom from an aromatic ring atom of an aromatic compound, which moiety has from 5 to 20 ring atoms, which include one or more heteroatoms. Preferably, each ring has from 5 to 7 ring atoms.

Examples of monocyclic heteroaryl groups include, but are not limited to, those derived from:

N₁: pyrrole (azole) (C₅), pyridine (azine) (C₆);

O₁: furan (oxole) (C₅);

S₁: thiophene (thiole) (C₅);

N₁O₁: oxazole (C₅), isoxazole (C₅), isoxazine (C₆);

N₂O₁: oxadiazole (furazan) (C₅);

N₃O₁: oxatriazole (C₅);

N₁S₁: thiazole (C₅), isothiazole (C₅);

N₂: imidazole (1,3-diazole) (C₅), pyrazole (1,2-diazole) (C₅), pyridazine (1,2-diazine) (C₆), pyrimidine (1,3-diazine) (C₆) (e.g., cytosine, thymine, uracil), pyrazine (1,4-diazine) (C₆);

N₃: triazole (C₅), triazine (C₆); and,

N₄: tetrazole (C₅).

Examples of heteroaryl groups which comprise fused rings, include, but are not limited to:

C₉ heteroaryl groups (with 2 fused rings) derived from benzofuran (O₁), isobenzofuran (O₁), indole (N₁), isoindole (N₁), indolizine (N₁), indoline (N₁), isoindoline (N₁), purine (N₄) (e.g., adenine, guanine), benzimidazole (N₂), indazole (N₂), benzoxazole (N₁O₁), benzisoxazole (N₁O₁), benzodioxole (O₂), benzofurazan (N₂O₁), benzotriazole (N₃), benzothiofuran (S₁), benzothiazole (N₁S₁), benzothiadiazole (N₂S);

C₁₀ heteroaryl groups (with 2 fused rings) derived from chromene (O₁), isochromene (O₁), chroman (O₁), isochroman (O₁), benzodioxan (O₂), quinoline (N₁), isoquinoline (N₁), quinolizine (N₁), benzoxazine (N₁O₁), benzodiazine (N₂), pyridopyridine (N₂), quinoxaline (N₂), quinazoline (N₂), cinnoline (N₂), phthalazine (N₂), naphthyridine (N₂), pteridine (N₄);

C₁₃ heteroaryl groups (with 3 fused rings) derived from carbazole (N₁), dibenzofuran (O₁), dibenzothiophene (S₁), carboline (N₂), perimidine (N₂), pyridoindole (N₂); and,

C₁₄ heteroaryl groups (with 3 fused rings) derived from acridine (N₁), xanthene (O₁), thioxanthene (S₁), oxanthrene (O₂), phenoxathiin (O₁S₁), phenazine (N₂), phenoxazine (N₁O₁), phenothiazine (N₁S₁), thianthrene (S₂), phenanthridine (N₁), phenanthroline (N₂), phenazine (N₂).

Heterocyclic groups (including heteroaryl groups) which have a nitrogen ring atom in the form of an —NH— group may be N-substituted, that is, as —NR—. For example, pyrrole may be N-methyl substituted, to give N-methypyrrole. Examples of N-substitutents include, but are not limited to C₁₋₇ alkyl, C₃₋₂₀ heterocyclyl, C₅₋₂₀ carboaryl, C₅₋₂₀ heteroaryl and acyl groups.

Heterocyclic groups (including heteroaryl groups) which have a nitrogen ring atom in the form of an —N═ group may be substituted in the form of an N-oxide, that is, as —N(→O)═ (also denoted —N⁺(→O⁻)═). For example, quinoline may be substituted to give quinoline N-oxide; pyridine to give pyridine N-oxide; benzofurazan to give benzofurazan N-oxide (also known as benzofuroxan).

Cyclic groups may additionally bear one or more oxo (═O) groups on ring carbon atoms. Monocyclic examples of such groups include, but are not limited to, those derived from:

C₅: cyclopentanone, cyclopentenone, cyclopentadienone;

C₆: cyclohexanone, cyclohexenone, cyclohexadienone;

O₁: furanone (C₅), pyrone (C₆);

N₁: pyrrolidone (pyrrolidinone) (C₅), piperidinone (piperidone) (C₆), piperidinedione (C₆);

N₂: imidazolidone (imidazolidinone) (C₅), pyrazolone (pyrazolinone) (C₅), piperazinone (C₆), piperazinedione (C₆), pyridazinone (C₆), pyrimidinone (C₆) (e.g., cytosine), pyrimidinedione (C₆) (e.g., thymine, uracil), barbituric acid (C₆);

N₁S₁: thiazolone (C₅), isothiazolone (C₅);

N₁O₁: oxazolinone (C₅).

Polycyclic examples of such groups include, but are not limited to, those derived from:

C₉: indenedione;

C₁₀: tetralone, decalone;

C₁₄: anthrone, phenanthrone;

N₁: oxindole (C₉);

O₁: benzopyrone (e.g., coumarin, isocoumarin, chromone) (C₁₀);

N₁O₁: benzoxazolinone (C₉), benzoxazolinone (C₁₀);

N₂: quinazolinedione (C₁₀);

N₄: purinone (C₉) (e.g., guanine).

Still more examples of cyclic groups which bear one or more oxo (═O) groups on ring carbon atoms include, but are not limited to, those derived from:

imides (—C(═O)—NR—C(═O)— in a ring), including but not limited to, succinimide (C₅), maleimide (C₅), phthalimide, and glutarimide (C₆);

lactones (cyclic esters, —)—C(═O)— in a ring), including, but not limited to, β-propiolactone, γ-butyrolactone, δ-valerolactone (2-piperidone), and ε-caprolactone;

lactams (cyclic amides, —NR—C(═O)— in a ring), including, but not limited to, β-propiolactam (C₄), γ-butyrolactam (2-pyrrolidone) (C₅), δ-valerolactam (C₆), and ε-caprolactam (C₇);

cyclic carbamates (—O—C(═O)—NR— in a ring), such as 2-oxazolidone (C₅);

cyclic ureas (—NR—C(═O)—NR— in a ring), such as 2-imidazolidone (C₅) and pyrimidine-2,4-dione (e.g., thymine, uracil) (C₆).

The above alkyl, heterocyclyl, carboaryl and heteroaryl groups, whether alone or part of another substituent, may themselves optionally be substituted with one or more groups selected from themselves and the additional substituents listed below, unless otherwise stated. Carboaryl and heteroaryl groups may also be substituted by alkoxylene groups as defined below. If the compounds of the present invention are of formulae IIa or IIb, it is preferred that the additional substituents are not selected from oxalamido, reverse carbamate and sulfonbisamino

Halo: —F, —Cl, —Br, and —I.

Hydroxy: —OH.

Ether: —OR, wherein R is an ether substituent, for example, a C₁₋₇ alkyl group (also referred to as a C₁₋₇ alkoxy group, discussed below), a C₃₋₂₀ heterocyclyl group (also referred to as a C₃₋₂₀ heterocyclyloxy group), or a C₅₋₂₀ aryl group (also referred to as a C₅₋₂₀ aryloxy group), preferably a C₁₋₇ alkyl group. The term C₅₋₂₀ aryl group encompasses both C₅₋₂₀ carboaryl and C₅₋₂₀ heteroaryl groups.

C₁₋₇ alkoxy: —OR, wherein R is a C₁₋₇ alkyl group. Examples of C₁₋₇ alkoxy groups include, but are not limited to, —OMe (methoxy), —OEt (ethoxy), —O(nPr) (n-propoxy), —O(iPr) (isopropoxy), —O(nBu) (n-butoxy), —O(sBu) (sec-butoxy), —O(iBu) (isobutoxy), and —O(tBu) (tert-butoxy).

Acetal: —CH(OR¹) (OR²), wherein R¹ and R² are independently acetal substituents, for example, a C₁₋₇ alkyl group, a C₃₋₂₀ heterocyclyl group, or a C₅₋₂₀ aryl group, preferably a C₁₋₇ alkyl group, or, in the case of a “cyclic” acetal group, R¹ and R², taken together with the two oxygen atoms to which they are attached, and the carbon atoms to which they are attached, form a heterocyclic ring having from 4 to 8 ring atoms. Examples of acetal groups include, but are not limited to, —CH(OMe)₂, —CH(OEt)₂, and —CH(OMe) (OEt).

Hemiacetal: —CH(OH) (OR¹), wherein R¹ is a hemiacetal substituent, for example, a C₁₋₇ alkyl group, a C₃₋₂₀ heterocyclyl group, or a C₅₋₂₀ aryl group, preferably a C₁₋₇ alkyl group. Examples of hemiacetal groups include, but are not limited to, —CH(OH) (OMe) and —CH(OH) (OEt).

Ketal: —CR(OR¹) (OR²), where R¹ and R² are as defined for acetals, and R is a ketal substituent other than hydrogen, for example, a C₁₋₇ alkyl group, a C₃₋₂₀ heterocyclyl group, or a C₅₋₂₀ aryl group, preferably a C₁₋₇ alkyl group. Examples ketal groups include, but are not limited to, —C(Me) (OMe)₂, —C(Me) (OEt)₂, —C(Me) (OMe) (OEt), —C(Et) (OMe)₂, —C(Et) (OEt)₂, and —C(Et) (OMe) (OEt).

Hemiketal: —CR(OH) (OR¹), where R¹ is as defined for hemiacetals, and R is a hemiketal substituent other than hydrogen, for example, a C₁₋₇ alkyl group, a C₃₋₂₀ heterocyclyl group, or a C₅₋₂₀ aryl group, preferably a C₁₋₇ alkyl group. Examples of hemiketal groups include, but are not limited to, —C(Me) (OH) (OMe), —C(Et) (OH) (OMe), —C(Me) (OH) (OEt), and —C(Et) (OH) (OEt).

Oxo (keto, -one): ═O.

Thione (thioketone): ═S.

Imino (imine): ═NR, wherein R is an imino substituent, for example, hydrogen, C₁₋₇ alkyl group, a C₃₋₂₀ heterocyclyl group, or a C₅₋₂₀ aryl group, preferably hydrogen or a C₁₋₇ alkyl group. Examples of ester groups include, but are not limited to, ═NH, ═NMe, ═NEt, and ═NPh.

Formyl (carbaldehyde, carboxaldehyde): —C(═O)H.

Acyl (keto): —C(═O)R, wherein R is an acyl substituent, for example, a C₁₋₇ alkyl group (also referred to as C₁₋₇ alkylacyl or C₁₋₇ alkanoyl), a C₃₋₂₀ heterocyclyl group (also referred to as C₃₋₂₀ heterocyclylacyl), or a C₅₋₂₀ aryl group (also referred to as C₅₋₂₀ arylacyl), preferably a C₁₋₇ alkyl group. Examples of acyl groups include, but are not limited to, —C(═O)CH₃ (acetyl), —C(═O)CH₂CH₃ (propionyl), —C(═O)C(CH₃)₃ (t-butyryl), and —C(═O)Ph (benzoyl, phenone).

Carboxy (carboxylic acid): —C(═O)OH.

Thiocarboxy (thiocarboxylic acid): —C(═S)SH.

Thiolocarboxy (thiolocarboxylic acid): —C(═O)SH.

Thionocarboxy (thionocarboxylic acid): —C(═S)OH.

Imidic acid: —C(═NH)OH.

Hydroxamic acid: —C(═O)NHOH.

Ester (carboxylate, carboxylic acid ester, oxycarbonyl): —C(═O)OR, wherein R is an ester substituent, for example, a C₁₋₇ alkyl group, a C₃₋₂₀ heterocyclyl group, or a C₅₋₂₀ aryl group, preferably a C₁₋₇ alkyl group. Examples of ester groups include, but are not limited to, —C(═O)OCH₃, —C(═O)OCH₂CH₃, —C(═O)OC(CH₃)₃, and —C(═O)OPh.

Acyloxy (reverse ester): —OC(═O)R, wherein R is an acyloxy substituent, for example, a C₁₋₇ alkyl group, a C₃₋₂₀ heterocyclyl group, or a C₅₋₂₀ aryl group, preferably a C₁₋₇alkyl group. Examples of acyloxy groups include, but are not limited to, —OC(═O)CH₃ (acetoxy), —OC(═O)CH₂CH₃, —OC(═O)C(CH₃)₃, —OC(═O) Ph, and —OC(═O)CH₂Ph.

Amido (carbamoyl, carbamyl, aminocarbonyl, carboxamide): —C(═O)NR¹R², wherein R¹ and R² are independently amino substituents, as defined for amino groups. Examples of amido groups include, but are not limited to, —C(═O)NH₂, —C(═O)NHCH₃, —C(═O)N(CH₃)₂, —C(═O)NHCH₂CH₃, and —C(═O)N(CH₂CH₃)₂, as well as amido groups in which R¹ and R², together with the nitrogen atom to which they are attached, form a heterocyclic structure as in, for example, piperidinocarbonyl, morpholinocarbonyl, thiomorpholinocarbonyl, and piperazinocarbonyl.

Acylamido (acylamino): —NR¹C(═O)R², wherein R¹ is an amide substituent, for example, hydrogen, a C₁₋₇ alkyl group, a C₃₋₂₀ heterocyclyl group, or a C₅₋₂₀ aryl group, preferably hydrogen or a C₁₋₇ alkyl group, and R² is an acyl substituent, for example, a C₁₋₇ alkyl group, a C₃₋₂₀ heterocyclyl group, or a C₅₋₂₀ aryl group, preferably hydrogen or a C₁₋₇alkyl group. Examples of acylamido groups include, but are not limited to, —NHC(═O)CH₃, —NHC(═O)CH₂CH₃, and —NHC(═O)Ph. R¹ and R² may together form a cyclic structure, as in, for example, succinimidyl, maleimidyl, and phthalimidyl: 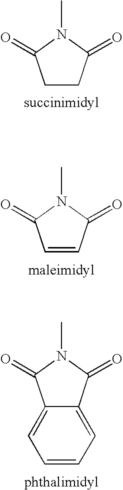
or possibly as in 3-hydro-isoindol-1-on-2-yl and 3-hydroxy-3-hydro-isoindol-1-on-2-yl:

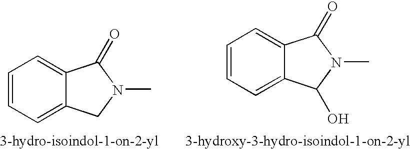

Oxalamido: —NR¹C(═O)C(═O)NR²NR³, wherein R² and R³ are independently amino substituents, as defined fro amino groups, and R¹ is a oxalamido substituent, for example, hydrogen, a C₁₋₇ alkyl group, a C₃₋₂₀ heterocyclyl group, or a C₅₋₂₀ aryl group, preferably hydrogen or a C₁₋₇ alkyl group. Examples of oxalamido groups include, but are not limited to, —NHCOCONH₂, —NHCOCONHMe, —NHCOCONHEt, —NHCOCONMe₂, —NHCOCONEt₂, —NMeCOCONH₂, —NMeCOCONHMe, —NMeCOCONHEt, —NMeCOCONMe₂, and —NMeCOCONEt₂.

Thioamido (thiocarbamyl): —C(═S)NR¹R², wherein R¹ and R² are independently amino substituents, as defined for amino groups. Examples of amido groups include, but are not limited to, —C(═S)NH₂, —C(═S)NHCH₃, —C(═S)N(CH₃)₂, and —C(═S)NHCH₂CH₃.

Ureido: —N(R¹)CONR²R³ wherein R² and R³ are independently amino substituents, as defined for amino groups, and R¹ is a ureido substituent, for example, hydrogen, a C₁₋₇ alkyl group, a C₃₋₂₀ heterocyclyl group, or a C₅₋₂₀ aryl group, preferably hydrogen or a C₁₋₇ alkyl group. Examples of ureido groups include, but are not limited to, —NHCONH₂, —NHCONHMe, —NHCONHEt, —NHCONMe₂, —NHCONEt₂, —NMeCONH₂, —NMeCONHMe, —NMeCONHEt, —NMeCONMe₂, and —NMeCONEt₂.

Carbamate: —NR¹C(═O)OR², wherein R¹ is an amide substituent, for example, hydrogen, a C₁₋₇ alkyl group, a C₃₋₂₀ heterocyclyl group, or a C₅₋₂₀ aryl group, preferably hydrogen or a C₁₋₇ alkyl group, and R² is an ester substituent, for example, a C₁₋₇ alkyl group, a C₃₋₂₀ heterocyclyl group, or a C₅₋₂₀ aryl group, preferably a C₁₋₇ alkyl group. Examples of carbamate groups include, but are not limited to, —NHC(═O)OCH₃, —NHC(═O)OCH₂CH₃, and —NHC(═O)OPh.

Reverse carbamate: —OC(═O)NR¹R², wherein R¹ and R² are independently amino substituents, as defined for amino groups. Examples of reverse carbamate groups include, but are not limited to, —OC(═O)NH₂, —OC(═O)NHCH₂CH₃, and —OC(═O)NHPh.

Guanidino: —NH—C(═NH)NH₂.

Tetrazolyl: a five membered aromatic ring having four nitrogen atoms and one carbon atom,

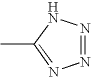

Amino: —NR¹R², wherein R¹ and R² are independently amino substituents, for example, hydrogen, a C₁₋₇ alkyl group (also referred to as C₁₋₇ alkylamino or di-C₁₋₇ alkylamino), a C₃₋₂₀ heterocyclyl group, or a C₅₋₂₀ aryl group, preferably H or a C₁₋₇ alkyl group, or, in the case of a “cyclic” amino group, R¹ and R², taken together with the nitrogen atom to which they are attached, form a heterocyclic ring having from 4 to 8 ring atoms. Amino groups may be primary (—NH₂), secondary (—NHR¹), or tertiary (—NHR¹R²), and in cationic form, may be quaternary (—⁺NR¹R²R³). Examples of amino groups include, but are not limited to, —NH₂, —NHCH₃, —NHC(CH₃)₂, —N(CH₃)₂, —N(CH₂CH₃)₂, and —NHPh. Examples of cyclic amino groups include, but are not limited to, aziridino, azetidino, pyrrolidino, piperidino, piperazino, morpholino, and thiomorpholino.

Imino: ═NR, wherein R is an imino substituent, for example, for example, hydrogen, a C₁₋₇ alkyl group, a C₃₋₂₀ heterocyclyl group, or a C₅₋₂₀ aryl group, preferably H or a C₁₋₇ alkyl group. Examples of imino groups include, but are not limited to, ═NH, ═NMe, and ═NEt.

Amidine (amidino): —C(═NR)NR₂, wherein each R is an amidine substituent, for example, hydrogen, a C₁₋₇ alkyl group, a C₃₋₂₀ heterocyclyl group, or a C₅₋₂₀ aryl group, preferably H or a C₁₋₇alkyl group. Examples of amidine groups include, but are not limited to, —C(═NH)NH₂, —C(═NH)NMe₂, and —C(═NMe)NMe₂.

Nitro: —NO₂.

Azido: —N₃.

Cyano (nitrile, carbonitrile): —CN.

Cyanato: —OCN.

Sulfhydryl (thiol, mercapto): —SH.

Thioether (sulfide): —SR, wherein R is a thioether substituent, for example, a C₁₋₇ alkyl group (also referred to as a C₁₋₇ alkylthio group), a C₃₋₂₀ heterocyclyl group, or a C₅₋₂₀ aryl group, preferably a C₁₋₇alkyl group. Examples of C₁₋₇ alkylthio groups include, but are not limited to, —SCH₃ and —SCH₂CH₃.

Sulfine (sulfinyl, sulfoxide): —S(═O)R, wherein R is a sulfine substituent, for example, a C₁₋₇ alkyl group, a C₃₋₂₀ heterocyclyl group, or a C₅₋₂₀ aryl group, preferably a C₁₋₇alkyl group. Examples of sulfine groups include, but are not limited to, —S(═O)CH₃ and —S(═O)CH₂CH₃.

Sulfone (sulfonyl): —S(═O)₂R, wherein R is a sulfone substituent, for example, a C₁₋₇ alkyl group, a C₃₋₂₀ heterocyclyl group, or a C₅₋₂₀ aryl group, preferably a C₁₋₇ alkyl group, including, for example, a fluorinated or perfluorinated C₁₋₇ alkyl group. Examples of sulfone groups include, but are not limited to, —S (═O)₂CH₃ (methanesulfonyl, mesyl) , —S(═O)₂CF₃ (triflyl), —S(═O)₂CH₂CH₃ (esyl), —S(═O)₂C₄F₉ (nonaflyl) , —S(═O)₂CH₂CF₃ (tresyl), —S(═O)₂CH₂CH₂NH₂ (tauryl), —S(═O)₂Ph (phenylsulfonyl, besyl), 4-methylphenylsulfonyl (tosyl), 4-chlorophenylsulfonyl (closyl), 4-bromophenylsulfonyl (brosyl), 4-nitrophenyl (nosyl), 2-naphthalenesulfonate (napsyl), and 5-dimethylamino-naphthalen-1-ylsulfonate (dansyl).

Sulfinic acid (sulfino): —S(═O)OH, —SO₂H.

Sulfonic acid (sulfo): —S(═O)₂OH, —SO₃H.

Sulfinate (sulfinic acid ester): —S(═O)OR; wherein R is a sulfinate substituent, for example, a C₁₋₇ alkyl group, a C₃₋₂₀ heterocyclyl group, or a C₅₋₂₀ aryl group, preferably a C₁₋₇ alkyl group. Examples of sulfinate groups include, but are not limited to, —S(═O)OCH₃ (methoxysulfinyl; methyl sulfinate) and —S(═O)OCH₂CH₃ (ethoxysulfinyl; ethyl sulfinate)

Sulfonate (sulfonic acid ester): —S(═O)₂OR, wherein R is a sulfonate substituent, for example, a C₁₋₇alkyl group, a C₃₋₂₀ heterocyclyl group, or a C₅₋₂₀ aryl group, preferably a C₁₋₇ alkyl group. Examples of sulfonate groups include, but are not limited to, —S(═O)₂OCH₃ (methoxysulfonyl; methyl sulfonate) and —S(═O)₂OCH₂CH₃ (ethoxysulfonyl; ethyl sulfonate).

Sulfinyloxy: —OS(═O)R, wherein R is a sulfinyloxy substituent, for example, a C₁₋₇ alkyl group, a C₃₋₂₀ heterocyclyl group, or a C₅₋₂₀ aryl group, preferably a C₁₋₇ alkyl group. Examples of sulfinyloxy groups include, but are not limited to, —OS(═O)CH₃ and —OS(═O)CH₂CH₃.

Sulfonyloxy: —OS(═O)₂R, wherein R is a sulfonyloxy substituent, for example, a C₁₋₇ alkyl group, a C₃₋₂₀ heterocyclyl group, or a C₅₋₂₀ aryl group, preferably a C₁₋₇ alkyl group. Examples of sulfonyloxy groups include, but are not limited to, —OS(═O)₂CH₃ (mesylate) and —OS(═O)₂CH₂CH₃ (esylate).

Sulfate: —OS(═O)₂OR; wherein R is a sulfate substituent, for example, a C₁₋₇ alkyl group, a C₃₋₂₀ heterocyclyl group, or a C₅₋₂₀ aryl group, preferably a C₁₋₇ alkyl group. Examples of sulfate groups include, but are not limited to, —OS(═O)₂OCH₃ and —SO(═O)₂OCH₂CH₃.

Sulfamyl (sulfamoyl; sulfinic acid amide; sulfinamide): —S(═O)NR¹R², wherein R¹ and R² are independently amino substituents, as defined for amino groups. Examples of sulfamyl groups include, but are not limited to, —S(═O)NH₂, —S(═O)NH(CH₃), —S(═O)N(CH₃)₂, —S(═O)NH(CH₂CH₃), —S(═O)N(CH₂CH₃)₂, and —S(═O)NHPh.

Sulfonamido (sulfinamoyl; sulfonic acid amide; sulfonamide): —S(═O)₂NR¹R², wherein R¹ and R² are independently amino substituents, as defined for amino groups. Examples of sulfonamido groups include, but are not limited to, —S(═O)₂NH₂, —S(═O)₂NH(CH₃), —S(═O)₂N(CH₃)₂; —S(═O)ZNH(CH₂CH₃), —S(═O)₂N(CH₂CH₃)₂, and —S(═O)₂NHPh.

Sulfamino: —NR²S(═O)₂OH, wherein R¹ is an amino substituent, as defined for amino groups. Examples of sulfamino groups include, but are not limited to, —NHS(═O)₂OH and —N(CH₃)S(═O)₂OH.

Sulfonamino: —NR¹S(═O)₂R, wherein R¹ is an amino substituent, as defined for amino groups, and R is a sulfonamino substituent, for example, a C₁₋₇ alkyl group, a C₃₋₂₀ heterocyclyl group, or a C₅₋₂₀ aryl group, preferably a C₁₋₇ alkyl group. Examples of sulfonamino groups include, but are not limited to, —NHS(═O)₂CH₃ and —N(CH₃)S(═O)₂C₆H₅.

Sulfonbisamino: —N(S(═O)₂R)₂, wherein R is a sulfonamino substituent, as defined for sulfonamino groups. Examples of sulfonbisamino groups include, but are not limited to, —N(S(═O)₂CH₃)₂ and —N(S(═O)₂C₆H₅)₂.

Sulfinamino: —NR¹S(═O)R, wherein R¹ is an amino substituent, as defined for amino groups, and R is a sulfinamino substituent, for example, a C₁₋₇ alkyl group, a C₃₋₂₀ heterocyclyl group, or a C₅₋₂₀ aryl group, preferably a C₁₋₇alkyl group. Examples of sulfinamino groups include, but are not limited to, —NHS(═O)CH₃ and —N(CH₃)S(═O)C₆H₅.

Further Groups

Alkoxylene: The term “alkoxylene” as used herein, pertains to a bidentate group which may be a substituent of an aryl group. It bonds to adjacent atoms of the aryl group, and may one or two carbon atoms in the chain between the oxygen atoms, as thus has the structure —O(CH₂)ₙO—, where n is either 1 or 2. The carbon atoms may bear any of the substituents listed above.

Includes Other Forms

Unless otherwise specified, included in the above are the well known ionic, salt, solvate, and protected forms of these substituents. For example, a reference to carboxylic acid (—COOH) also includes the anionic (carboxylate) form (—COO⁻), a salt or solvate thereof, as well as conventional protected forms.

Similarly, a reference to an amino group includes the protonated form (—N⁺HR¹R²), a salt or solvate of the amino group, for example, a hydrochloride salt, as well as conventional protected forms of an amino group. Similarly, a reference to a hydroxyl group also includes the anionic form (—O⁻), a salt or solvate thereof, as well as conventional protected forms of a hydroxyl group.

Isomers, Salts, Solvates, Protected Forms, and Prodrugs

Certain compounds may exist in one or more particular geometric, optical, enantiomeric, diasteriomeric, epimeric, stereoisomeric, tautomeric, conformational, or anomeric forms, including but not limited to, cis- and trans-forms; E- and Z-forms; c-, t-, and r-forms; endo- and exo-forms; R-, S-, and meso-forms; D- and L-forms; d- and l-forms; (+) and (−) forms; keto-, enol-, and enolate-forms; syn- and anti-forms; synclinal- and anticlinal-forms; α- and β-forms; axial and equatorial forms; boat-, chair-, twist-, envelope-, and halfchair-forms; and combinations thereof, hereinafter collectively referred to as “isomers” (or “isomeric forms”).

Note that, except as discussed below for tautomeric forms, specifically excluded from the term “isomers,” as used herein, are structural (or constitutional) isomers (i.e., isomers which differ in the connections between atoms rather than merely by the position of atoms in space). For example, a reference to a methoxy group, —OCH₃, is not to be construed as a reference to its structural isomer, a hydroxymethyl group, —CH₂OH. Similarly, a reference to ortho-chlorophenyl is not to be construed as a reference to its structural isomer, meta-chlorophenyl. However, a reference to a class of structures may well include structurally isomeric forms falling within that class (e.g., C₁₋₇alkyl includes n-propyl and iso-propyl; butyl includes n-, iso-, sec-, and tert-butyl; methoxyphenyl includes ortho-, meta-, and para-methoxyphenyl).

The above exclusion does not pertain to tautomeric forms, for example, keto-, enol-, and enolate-forms, as in, for example, the following tautomeric pairs: keto/enol (illustrated below), imine/enamine, amide/imino alcohol, amidine/amidine, nitroso/oxime, thioketone/enethiol, and nitro/aci-nitro.

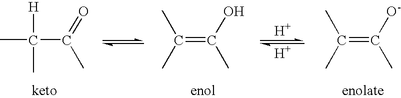

Note that specifically included in the term “isomer” are compounds with one or more isotopic substitutions. For example, H may be in any isotopic form, including ¹H, ²H (D), and ³H (T); C may be in any isotopic form, including ¹²C, ¹³C, and ¹⁴C; O may be in any isotopic form, including ¹⁶O and ¹⁸O; and the like.

Unless otherwise specified, a reference to a particular compound includes all such isomeric forms, including (wholly or partially) racemic and other mixtures thereof. Isomeric forms substantially free, i.e. associated with less than 5%, preferably less than 2%, in particular less than 1%, of the other isomeric form are also envisaged. Methods for the preparation (e.g., asymmetric synthesis) and separation (e.g., fractional crystallisation and chromatographic means) of such isomeric forms are either known in the art or are readily obtained by adapting the methods taught herein, or known methods, in a known manner.

Unless otherwise specified, a reference to a particular compound also includes ionic, salt, solvate, and protected forms of thereof, for example, as discussed below.

It may be convenient or desirable to prepare, purify, and/or handle a corresponding salt of the active compound, for example, a pharmaceutically-acceptable salt. Examples of pharmaceutically acceptable salts are discussed in Berge et al., 1977, “Pharmaceutically Acceptable Salts,” J. Pharm. Sci., Vol. 66, pp. 1-19.

For example, if the compound is anionic, or has a functional group which may be anionic (e.g., —COOH may be —COO⁻), then a salt may be formed with a suitable cation. Examples of suitable inorganic cations include, but are not limited to, alkali metal ions such as Na⁺ and K⁺, alkaline earth cations such as Ca²⁺ and Mg²⁺, and other cations such as Al³⁺. Examples of suitable organic cations include, but are not limited to, ammonium ion (i.e., NH₄⁺) and substituted ammonium ions (e.g., NH₃R⁺, NH₂R₂⁺, NHR₃⁺, NR₄⁺) Examples of some suitable substituted ammonium ions are those derived from: ethylamine, diethylamine, dicyclohexylamine, triethylamine, butylamine, ethylenediamine, ethanolamine, diethanolamine, piperazine, benzylamine, phenylbenzylamine, choline, meglumine, and tromethamine, as well as amino acids, such as lysine and arginine. An example of a common quaternary ammonium ion is N(CH₃)₄⁺.

If the compound is cationic, or has a functional group which may be cationic (e.g., —NH₂ may be —NH₃⁺), then a salt may be formed with a suitable anion. Examples of suitable inorganic anions include, but are not limited to, those derived from the following inorganic acids: hydrochloric, hydrobromic, hydroiodic, sulfuric, sulfurous, nitric, nitrous, phosphoric, and phosphorous.

Examples of suitable organic anions include, but are not limited to, those derived from the following organic acids: 2-acetyoxybenzoic, acetic, ascorbic, aspartic, benzoic, camphorsulfonic, cinnamic, citric, edetic, ethanedisulfonic, ethanesulfonic, fumaric, glucheptonic, gluconic, glutamic, glycolic, hydroxymaleic, hydroxynaphthalene carboxylic, isethionic, lactic, lactobionic, lauric, maleic, malic, methanesulfonic, mucic, oleic, oxalic, palmitic, pamoic, pantothenic, phenylacetic, phenylsulfonic, propionic, pyruvic, salicylic, stearic, succinic, sulfanilic, tartaric, toluenesulfonic, and valeric. Examples of suitable polymeric organic anions include, but are not limited to, those derived from the following polymeric acids: tannic acid, carboxymethyl cellulose.

It may be convenient or desirable to prepare, purify, and/or handle a corresponding solvate of the active compound. The term “solvate” is used herein in the conventional sense to refer to a complex of solute (e.g., active compound, salt of active compound) and solvent. If the solvent is water, the solvate may be conveniently referred to as a hydrate, for example, a mono-hydrate, a di-hydrate, a tri-hydrate, etc.

It may be convenient or desirable to prepare, purify, and/or handle the active compound in a chemically protected form. The term “chemically protected form” is used herein in the conventional chemical sense and pertains to a compound in which one or more reactive functional groups are protected from undesirable chemical reactions under specified conditions (e.g., pH, temperature, radiation, solvent, and the like). In practice, well known chemical methods are employed to reversibly render unreactive a functional group, which otherwise would be reactive, under specified conditions. In a chemically protected form, one or more reactive functional groups are in the form of a protected or protecting group (also known as a masked or masking group or a blocked or blocking group). By protecting a reactive functional group, reactions involving other unprotected reactive functional groups can be performed, without affecting the protected group; the protecting group may be removed, usually in a subsequent step, without substantially affecting the remainder of the molecule. See, for example, Protective Groups in Organic Synthesis (T. Green and P. Wuts; 3rd Edition; John Wiley and Sons, 1999).

A wide variety of such “protecting,” “blocking,” or “masking” methods are widely used and well known in organic synthesis. For example, a compound which has two nonequivalent reactive functional groups, both of which would be reactive under specified conditions, may be derivatized to render one of the functional groups “protected,” and therefore unreactive, under the specified conditions; so protected, the compound may be used as a reactant which has effectively only one reactive functional group. After the desired reaction (involving the other functional group) is complete, the protected group may be “deprotected” to return it to its original functionality.

For example, a hydroxy group may be protected as an ether (—OR) or an ester (—OC(═O)R), for example, as: a t-butyl ether; a benzyl, benzhydryl (diphenylmethyl), or trityl (triphenylmethyl) ether; a trimethylsilyl or t-butyldimethylsilyl ether; or an acetyl ester (—OC(═O)CH₃, —OAc).

For example, an aldehyde or ketone group may be protected as an acetal (R—CH(OR)₂) or ketal (R₂C(OR)₂), respectively, in which the carbonyl group (>C═O) is converted to a diether (>C(OR)₂), by reaction with, for example, a primary alcohol. The aldehyde or ketone group is readily regenerated by hydrolysis using a large excess of water in the presence of acid.

For example, an amine group may be protected, for example, as an amide (—NRCO—R) or a urethane (—NRCO—OR), for example, as: a methyl amide (—NHCO—CH₃); a benzyloxy amide (—NHCO—OCH₂C₆H₅, —NH—Cbz); as a t-butoxy amide (—NHCO—OC(CH₃)₃, —NH-Boc); a 2-biphenyl-2-propoxy amide (—NHCO—OC(CH₃)₂C₆H₄C₆H₅, —NH-Bpoc), as a 9-fluorenylmethoxy amide (—NH-Fmoc), as a 6-nitroveratryloxy amide (—NH-Nvoc), as a 2-trimethylsilylethyloxy amide (—NH-Teoc), as a 2,2,2-trichloroethyloxy amide (—NH-Troc), as an allyloxy amide (—NH-Alloc), as a 2(-phenylsulphonyl)ethyloxy amide (—NH-Psec); or, in suitable cases (e.g., cyclic amines), as a nitroxide radical (>N—O.).

For example, a carboxylic acid group may be protected as an ester for example, as: an C₁₋₇ alkyl ester (e.g., a methyl ester; a t-butyl ester); a C₁₋₇ haloalkyl ester (e.g., a C₁₋₇ trihaloalkyl ester); a triC₁₋₇ alkylsilyl-C₁₋₇alkyl ester; or a C₅₋₂₀ aryl-C₁₋₇ alkyl ester (e.g., a benzyl ester; a nitrobenzyl ester); or as an amide, for example, as a methyl amide.

For example, a thiol group may be protected as a thioether (—SR), for example, as: a benzyl thioether; an acetamidomethyl ether (—S—CH₂NHC(═O)CH₃).

It may be convenient or desirable to prepare, purify, and/or handle the active compound in the form of a prodrug. The term “prodrug,” as used herein, pertains to a compound which, when metabolised (e.g., in vivo), yields the desired active compound.

Typically, the prodrug is inactive, or less active than the active compound, but may provide advantageous handling, administration, or metabolic properties.

For example, some prodrugs are esters of the active compound (e.g., a physiologically acceptable metabolically labile ester). During metabolism, the ester group (—C(═O)OR) is cleaved to yield the active drug. Such esters may be formed by esterification, for example, of any of the carboxylic acid groups (—C(═O)OH) in the parent compound, with, where appropriate, prior protection of any other reactive groups present in the parent compound, followed by deprotection if required.

Examples of such metabolically labile esters include those of the formula —C(═O)OR wherein R is:

C₁₋₇alkyl

(e.g., —Me, -Et, -nPr, -iPr, -nBu, -sBu, -iBu, -tBu);

C₁₋₇aminoalkyl

(e.g., aminoethyl; 2-(N,N-diethylamino)ethyl;

2-(4-morpholino)ethyl); and

acyloxy-C₁₋₇alkyl

(e.g., acyloxymethyl;

acyloxyethyl;

pivaloyloxymethyl;

acetoxymethyl;

1-acetoxyethyl;

1-(1-methoxy-1-methyl)ethyl-carbonxyloxyethyl;

1-(benzoyloxy)ethyl; isopropoxy-carbonyloxymethyl;

1-isopropoxy-carbonyloxyethyl; cyclohexyl-carbonyloxymethyl;

1-cyclohexyl-carbonyloxyethyl;

cyclohexyloxy-carbonyloxymethyl;

1-cyclohexyloxy-carbonyloxyethyl;

(4-tetrahydropyranyloxy) carbonyloxymethyl;

1-(4-tetrahydropyranyloxy)carbonyloxyethyl;

(4-tetrahydropyranyl)carbonyloxymethyl; and

1-(4-tetrahydropyranyl)carbonyloxyethyl).

Also, some prodrugs are activated enzymatically to yield the active compound, or a compound which, upon further chemical reaction, yields the active compound (for example, as in ADEPT, GDEPT, LIDEPT, etc.). For example, the prodrug may be a sugar derivative or other glycoside conjugate, or may be an amino acid ester derivative.

Preferences

The following preferences apply to each aspect of the present invention, and preferred compounds may be different for different aspects. The following preferences for each group may be combined in any way with preferences for other groups.

In some embodiments, it is preferred that the molecular weight of the compound is less than 1000, and more preferably less than 750, although the molecular weight may be less than 700, 650, 600, 550, 525 or even 500.

—X═Y—

It is preferred that —X═Y— is —CR²═N—, i.e. that the compounds are of formula Ib.

R⁵

R⁵ is preferably selected from R^{5′}, halo, NHR^{5′}, OR^{5′}, SR^{5′}, wherein R^{5′} is H or C₁₋₃ alkyl (optionally substituted by halo, NH₂, OH, SH). Of these groups, H, NHR^{5′} (more preferably NH₂), OH, SH and halo (more preferably F or Cl) are more preferred, with H and NH₂ being the most preferred. If the compound is a pyridine then preferably R⁵ is NH₂, and if the compound is a pyrazine preferably R⁵ is H.

R¹

R¹ is preferably selected from H, NRR′, NHC(═O)R, NHC(═O)NRR′, and NH₂SO₂R, and more preferably from H and NRR′, or from H and NH₂. R¹ is most preferably H.

In some embodiments, R¹ is preferably selected from NHC(═O)R, NHC(═O)NRR′, and NH₂SO₂R.

R² and R³

R² and R³ (where present) are preferably independently selected from H, halo, amino, hydroxy and thio, and more preferably from H and halo. If only one of R² and R³ is a substituent, then R² is the preferred substituent.

R⁴

R⁴ is preferably an optionally substituted C₅₋₁₀ aryl group, more preferably either a C₅₋₁₀ carboaryl group or a C₅₋₁₀ heteroaryl group having one or two nitrogen ring atoms, for example, naphthyl, phenyl, indole, quinoline, isoquinoline, tetrahydroquinoline, tetrahydroisoquinoline, pyridine, phthalazine, tetrahydrophthalazine, quinazoline and tetrahydroquinazoline.

In one embodiment R⁴ is an optionally substituted C₅₋₁₀ carboaryl group, and more preferably an optionally substituted phenyl or napthyl group.

If R⁴ is a napthyl group it is preferably unsubstituted, and may be in any configuration, with napth-1-yl being preferred.

If R⁴ is a phenyl group, then it is preferably substituted, more preferably with one or two substituents.

These are preferably selected from halo (more preferably F and Cl) , ether (more preferably C₁₋₇ alkoxy, and in particular —OMe, and arylalkoxy, and in particular benzyloxy), C₁₋₇ alkyl (more preferably C₁₋₄ alkyl, and in particular —Me, and —CF₃), C₅₋₂₀ aryl groups (more preferably C₅₋₁₀ carboaryl or heteroaryl groups), amido, acylamido, ureido, carbamate and reverse carbamate. Alkoxylene groups linked to adjacent atoms are also preferred.

In particular amido, acylamido, ureido, carbamate and reverse carbamate groups are preferred, optionally in combination with a halo group, which is preferably para to the former groups. The former groups are preferably in the 3-position.

If there is one substituent, the ortho and meta positions are preferred, with the meta position being the most preferred. If two substituents are present, it may be preferred that neither is in the para position, unless one is F, when this is preferred to be in the para position.

In another embodiment, R⁴ is preferably a bicyclic aryl group, where the second ring can be aromatic or non-aromatic (partially or fully saturated). Such groups include napthyl, indole, oxindole, quinoline, isoquinoline, tetrahydroquinoline and tetrahydroisoquinoline.

In a further embodiment, R⁴ is preferably a 2,6-dichlorophenyl group. When R⁴ is this group, R⁵ is preferably H and R¹ is preferably selected from NHR, NHC(═O)R and NHC(═O)NRR′, and more preferably NHC(═O)NRR′.

As discussed above, preferred compounds of the present invention are of formulae IIa and IIb:

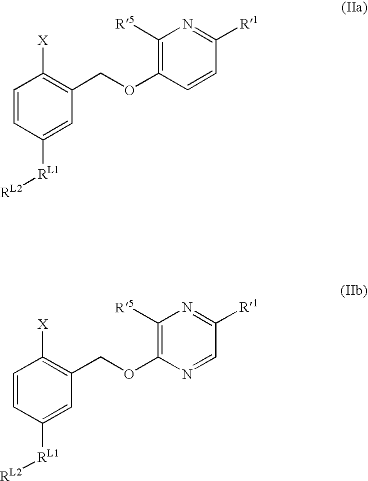

The preferences for compounds of formula IIa are as follows:

R′¹

R′¹ is preferably selected from H and NR^{C1}R^{C2}, and more preferably from H and NHR^{C1}. If R′¹ is NHR^{C1}, then R^{C1} is preferably C₁₋₄ alkyl (more preferably C₁₋₂ alkyl) which may be, and is more preferably, substituted by OH, NH₂, C₅₋₂₀ carboaryl (more preferably C₅₋₁₀ carboaryl, e.g. phenyl) , and C₅₋₂₀ heteroaryl (more preferably C₅₋₁₀ heteroaryl, e.g. pyridyl). Examples of preferred R′¹ groups include, but are not limited to, —NH—C₂H₄—OH and —NH—CH₂—C₆H₅.

In some embodiments, R′¹ is preferably selected from NHC(═O)R^{C1}, NHC(═O)NR^{C1}R^{C2}, and NH₂SO₂R^{C1}.

R′⁵

R′⁵ is preferably H.

X

X is preferably halo, and more preferably F or Cl, with Cl being most preferred.

R^{L1}

R^{L1} is preferably selected from —NH—C(═O)—, —NH—C(═O)—NH— and —NH—C(═O)—O—, more preferably from —NH—C(═O)— and —NH—C(═O)—NH— and is most preferably —NH—C(═O)—.

In some embodiments, it is preferred that R is not —NH—C(═O)—NH—.

R^{L2}

R^{L2} is preferably a C₅₋₂₀ carboaryl or C₅₋₂₀ heteroaryl group, more preferably a C₅₋₂₀ carboaryl group when R^{L1} is —NH—C(═O)— and more preferably a C₅₋₂₀ heteroaryl group when R^{L1} is —NH—C(═O)—NH—.

Particularly preferred are monocyclic carboaryl and heteroaryl groups. If R^{L2} is a carboaryl group, it is preferably phenyl. If R^{L2} is a heteroaryl group it is preferably comprises at least one nitrogen ring atom (e.g. pyrrole, pyridine, thiazole, pyrazole, triazole), and is more preferably pyridine, thiazole or pyrazole, with pyrazole being the most preferred. Heteroaryl groups may be formed into a moeity by removing a hydrogen from a carbon or hetero ring atom, with the preference being for removal from a carbon ring atom.

The C₅₋₂₀ carboaryl or C₅₋₂₀ heteroaryl group is preferably substituted by one or more substituent groups, more preferably one or two substituents.

When R^{L2} is a six membered ring, it is preferred that at least one substituent group is in the meta position (i.e. β to attachment to R^{L1}), and if there are two substituents these are both preferably in the meta positions.

When R^{L2} is a five membered ring, it is preferred that at least one substituent group is either α or γ to attachment to R^{L1}, with the γ position being preferred.

The substituents are preferably selected from halo (more preferably F and Cl), amino (more preferably cyclic amino groups, and in particular morpholino), C₁₋₇ alkyl (more preferably C₁₋₄ alkyl, and in particular —Me, -t-Bu and —CF₃) , C₅₋₂₀ carboaryl groups (more preferably C₅₋₁₀ carboaryl groups, and in particular, phenyl) and C₅₋₂₀ heteroaryl groups (more preferably C₅₋₁₀ heteroaryl groups).

Compounds of the present invention of formula IIa include N-[4-Chloro-3-(pyridin-3-yloxymethyl)-phenyl]-2-morpholin-4-yl-isonicotinamide (44), N-[4-Chloro-3-(pyridin-3-yloxymethyl)-phenyl]-3-fluoro-5-morpholin-4-yl-benzamide (49), N-[4-Chloro-3-(pyridin-3-yloxymethyl)-phenyl]-3-fluoro-benzamide (50), N-[4-Chloro-3-(pyridin-3-yloxymethyl)-phenyl]-benzamide (52), N-[4-Chloro-3-(pyridin-3-yloxymethyl)-phenyl]-isonicotinamide (53), N-[3-(2-Amino-pyridin-3-yloxymethyl)-4-chloro-phenyl]-benzamide (57), N-[4-Fluoro-3-(pyridin-3-yloxymethyl)-phenyl]-benzamide (59), 3-Fluoro-N-[4-fluoro-3-(pyridin-3-yloxymethyl)-phenyl]-benzamide (60), 1-[4-Chloro-3-(pyridin-3-yloxymethyl)-phenyl]-3-phenyl-urea (61), 3-Fluoro-N-[4-fluoro-3-(pyridin-3-yloxymethyl)-phenyl]-5-morpholin-4-yl-benzamide (62), [4-Chloro-3-(pyridin-3-yloxymethyl)-phenyl]-urea (63), 1-(5-tert-Butyl-2-phenyl-2H-yrazol-3-yl)-3-[4-chloro-3-(pyridin-3-yloxymethyl)-phenyl]-urea (64), 3-tert-Butyl-N-[4-chloro-3-(pyridin-3-yloxymethyl)-phenyl]-benzamide (65), N-[3-(Pyridin-3-yloxymethyl)-phenyl]-benzamide (66), 3-Fluoro-5-morpholin-4-yl-N-[3-(pyridin-3-yloxymethyl)-phenyl]-benzamide (67), N-[4-Chloro-3-(pyridin-3-yloxymethyl)-phenyl]-3-trifluoromethyl-benzamide (69), 3-Chloro-N-[4-chloro-3-(pyridin-3-yloxymethyl)-phenyl]-benzamide (70), 1-(5-tert-Butyl-2H-pyrazol-3-yl)-3-[4-chloro-3-(pyridin-3-yloxymethyl)-phenyl]-urea (71), 6-Morpholin-4-yl-pyrazine-2-carboxylic acid [4-fluoro-3-(pyridin-3-yloxymethyl)-phenyl]-amide (75), N-{4-Chloro-3-[6-(2-hydroxy-ethylamino)-pyridin-3-yloxymethyl]-phenyl}-3-fluoro-5-morpholin-4-yl-benzamide (76), N-[3-(6-Benzylamino-pyridin-3-yloxymethyl)-4-chloro-phenyl]-3-fluoro-5-morpholin-4-yl-benzamide (77), 1-(2-tert-Butyl-phenyl)-3-[4-fluoro-3-(pyridin-3-yloxymethyl)-phenyl]-urea (78), [4-Chloro-3-(pyridin-3-yloxymethyl)-phenyl]-carbamic acid phenyl ester (79) and 1-[4-Fluoro-3-(pyridin-3-yloxymethyl)-phenyl]-3-(5-isopropyl-[1,3,4]thiadiazol-2-yl)-urea (81).

Of these compounds, the following are preferred embodiments of compounds of formula IIa: N-[4-Chloro-3-(pyridin-3-yloxymethyl)-phenyl]-2-morpholin-4-yl-isonicotinamide (44), N-[4-Chloro-3-(pyridin-3-yloxymethyl)-phenyl]-3-fluoro-5-morpholin-4-yl-benzamide (49), 3-Fluoro-N-[4-fluoro-3-(pyridin-3-yloxymethyl)-phenyl]-5-morpholin-4-yl-benzamide (62), 1-(5-tert-Butyl-2-phenyl-2H-pyrazol-3-yl)-3-[4-chloro-3-(pyridin-3-yloxymethyl)-phenyl]-urea (64), 3-tert-Butyl-N-[4-chloro-3-(pyridin-3-yloxymethyl)-phenyl]-benzamide (65), N-{4-Chloro-3-[6-(2-hydroxy-ethylamino)-pyridin-3-yloxymethyl]-phenyl}-3-fluoro-5-morpholin-4-yl-benzamide (76), and N-[3-(6-Benzylamino-pyridin-3-yloxymethyl)-4-chloro-phenyl]-3-fluoro-5-morpholin-4-yl-benzamide (77).

The preferences for compounds of formula IIb are as follows:

R′¹

R′¹ is preferably selected from H and NR^{C1}R^{C2}, and more preferably from H and NHR^{C1}. If R′¹ is NHR^{C1}, then R^{C1} is preferably C₁₋₄ alkyl (more preferably C₁₋₂ alkyl) which may be, and is more preferably, substituted by OH, NH₂, C₅₋₂₀ carboaryl (more preferably C₅₋₁₀ carboaryl, e.g. phenyl), and C₅₋₂₀ heteroaryl (more preferably C₅₋₁₀ heteroaryl, e.g. pyridyl). Examples of preferred R′¹ groups include, but are not limited to, H, —NH—C₂H₄—OH and —NH—CH₂—C₆H₅.

In some embodiments, R′¹ is preferably selected from NHC(═O)R^{C1}, NHC(═O)NR^{C1}R^{C2}, and NH₂SO₂R^{C1}.

R′⁵

R′⁵ is preferably H.

X

X is preferably halo, and more preferably F or Cl, with F being most preferred.

R^{L1}

R^{L1} is preferably selected from —NH—C(═O)—, —NH—C(═O)—NH— and —NH— C(═O)—O—, more preferably from —NH—C(═O)— and —NH—C(═O)—NH— and is most preferably —NH—C(═O)—NH—.

In some embodiments, it is preferred that R^{L1} is not —NH—C(═O)—NH—.

R^{L2}

R^{L2} is preferably a C₅₋₂₀ carboaryl or C₅₋₂₀ heteroaryl group, more preferably a C₅₋₂₀ carboaryl group when R is —NH—C(═O)—, and more preferably a C₅₋₂₀ heteroaryl group when R^{L1} is —NH—C(═O)—NH—.

Particularly preferred are monocyclic carboaryl and heteroaryl groups. If R^{L2} is a carboaryl group, it is preferably phenyl. If R^{L2} is a heteroaryl group it is preferably comprises at least one nitrogen ring atom (e.g. pyrrole, pyridine, isoxazole, thiazole, pyrazole, thiadiazole, oxadiazole, triazole), and is more preferably pyridine, thiazole, thiadiazole or pyrazole, with pyrazole being the most preferred. Heteroaryl groups may be formed into a moiety by removing a hydrogen from a carbon or hetero ring atom, with the preference being for removal from a carbon ring atom.

The C₅₋₂₀ carboaryl or C₅₋₂₀ heteroaryl group is preferably substituted by one or more substituent groups, more preferably one or two substituents.

When R^{L2} is a six membered ring, it is preferred that at least one substituent group is in the meta position (i.e. β to attachment to R^{L1}), and if there are two substituents these are both preferably in the meta positions.

When R^{L2} is a five membered ring, it is preferred that at least one substituent group is either α or γ to attachment to R^{L1}, with the γ position being preferred.

When R^{L2} is a nitrogen containing five membered heteroaryl group, it is preferred that one of the nitrogen atoms, and preferably that α to attachment to R^{L1}, is substituted.

The substituents are preferably selected from halo (more preferably F and Cl), amino (more preferably cyclic amino groups, and in particular morpholino), C₁₋₇ alkyl (more preferably C₁₋₄ alkyl, and in particular —Me, -i-Pr, cyclopropyl, -t-Bu and —CF₃), C₃₋₂₀ heterocyclyl groups (more preferably C₃₋₇ heterocyclyl groups, and in particular oxolane and oxane), C₅₋₂₀ carboaryl groups (more preferably C₅₋₁₀ carboaryl groups, and in particular, phenyl), C₅₋₂₀ heteroaryl groups (more preferably C₅₋₁₀ heteroaryl groups, and in particular, pyridine, pyrazine, pyrimidine, thiazole), carboarylalkyl groups (more preferably benzyl) and carboaryloxy groups (more preferably phenyloxy).

Compounds of the present invention of formula IIb include N-[4-Chloro-3-(pyrazin-2-yloxymethyl)-phenyl]-benzamide (92), N-[4-Chloro-3-(pyrazin-2-yloxymethyl)-phenyl]-2-morpholin-4-yl-isonicotinamide (93), N-[4-Chloro-3-(pyrazin-2-yloxymethyl)-phenyl]-3-fluoro-5-morpholin-4-yl-benzamide (94), 1-(5-Cyclopropylmethyl-[1,3,4]thiadiazol-2-yl)-3-[4-fluoro-3-(pyrazin-2-yloxymethyl)-phenyl]-urea (96), 1-[4-Fluoro-3-(pyrazin-2-yloxymethyl)-phenyl]-3-(5-isopropyl-[1,3,4]thiadiazol-2-yl)-urea (97), [4-Fluoro-3-(pyrazin-2-yloxymethyl)-phenyl]-carbamic acid 3-trifluoromethyl-phenyl ester (99), 1-(4-tert-Butyl-thiazol-2-yl)-3-[4-fluoro-3-(pyrazin-2-yloxymethyl)-phenyl]-urea (100) 4-tert-Butyl-N-[4-fluoro-3-(pyrazin-2-yloxymethyl)-phenyl]-benzamide (101), N-[4-Fluoro-3-(pyrazin-2-yloxymethyl)-phenyl]-3-phenoxy-benzamide (102), 3-tert-Butyl-N-[4-fluoro-3-(pyrazin-2-yloxymethyl)-phenyl]-benzamide (103), 6-(3H-Benzotriazol-1-yloxy)-2-chloro-pyrimidine-4-carboxylic acid [4-fluoro-3-(pyrazin-2-yloxymethyl)-phenyl]-amide (104), 2-Chloro-6-methoxy-pyrimidine-4-carboxylic acid [4-fluoro-3-(pyrazin-2-yloxymethyl)-phenyl]-amide (105), 1-(5-tert-Butyl-2-phenyl-2H-pyrazol-3-yl)-3-[4-fluoro-3-(pyrazin-2-yloxymethyl)-phenyl]-urea (106), Phenyl-carbamic acid 3-(pyrazin-2-yloxymethyl)-phenyl ester (107), 1-[4-Fluoro-3-(pyrazin-2-yloxymethyl)-phenyl]-3-(5-phenyl-[1,3,4]thiadiazol-2-yl)-urea (115), 1-(4,6-Dimethyl-benzothiazol-2-yl)-3-[4-fluoro-3-(pyrazin-2-yloxymethyl)-phenyl]-urea (116), 1-[5-(4-Chloro-phenyl)-thiazol-2-yl]-3-[4-fluoro-3-(pyrazin-2-loxymethyl)-phenyl]-urea (117), 1-[4-Fluoro-3-(pyrazin-2-yloxymethyl)-phenyl]-3-(5-phenyl-1H-pyrazol-3-yl)-urea (118), 1-[4-Fluoro-3-(pyrazin-2-yloxymethyl)-phenyl]-3-(4-phenyl-1H-pyrazol-3-yl)-urea (119), 1-[4-Fluoro-3-(pyrazin-2-yloxymethyl)-phenyl]-3-[5-(tetrahydro-furan-2-yl)-[1,3,4]thiadiazol-2-yl]-urea (120), 1-(5-Benzyl-[1,3,4]thiadiazol-2-yl)-3-[4-fluoro-3-(pyrazin-2-yloxymethyl)-phenyl]-urea (121), 3-Methyl-5-phenyl-isoxazole-4-carboxylic acid [4-fluoro-3-(pyrazin-2-yloxymethyl)-phenyl]-amide (122), 1-[4-Fluoro-3-(pyrazin-2-yloxymethyl)-phenyl]-3-(4-phenyl-thiazol-2-yl)-urea (123), 5-(2-Methyl-thiazol-4-yl)-isoxazole-3-carboxylic acid [4-fluoro-3-(pyrazin-2-yloxymethyl)-phenyl]-amide (124), 1-[5-tert-Butyl-2-(2,4-difluoro-phenyl)-2H-pyrazol-3-yl]-3-[4-fluoro-3-(pyrazin-2-yloxymethyl)-phenyl]-urea (125), 5-Phenyl-[1,3,4]oxadiazole-2-carboxylic acid [4-fluoro-3-(pyrazin-2-yloxymethyl)-phenyl]-amide (126), 1-[5-tert-Butyl-2-(4-chloro-phenyl)-2H-pyrazol-3-yl]-3-[4-fluoro-3-(pyrazin-2-yloxymethyl)-phenyl]-urea (127), 1-[5-(4-Chloro-phenyl)-2-phenyl-2H-pyrazol-3-yl]-3-[4-fluoro-3-(pyrazin-2-yloxymethyl)-phenyl]-urea (128), 1-(5-tert-Butyl-2-p-tolyl-2H-pyrazol-3-yl)-3-[4-fluoro-3-(pyrazin-2-yloxymethyl)-phenyl]-urea (130), Naphthalene-2-carboxylic acid [4-fluoro-3-(pyrazin-2-yloxymethyl)-phenyl]-amide (131), 1-[5-(4-Chloro-phenyl)-2-(4-fluoro-phenyl)-2H-pyrazol-3-yl]-3-[4-fluoro-3-(pyrazin-2-yloxymethyl)-phenyl]-urea (132), Biphenyl-4-carboxylic acid [4-fluoro-3-(pyrazin-2-yloxymethyl)-phenyl]-amide (133), 1-(2,5-Diphenyl-2H-pyrazol-3-yl)-3-[4-fluoro-3-(pyrazin-2-yloxymethyl)-phenyl]-urea (134), 2-Benzyl-5-tert-butyl-2H-pyrazole-3-carboxylic acid [4-fluoro-3-(pyrazin-2-yloxymethyl)-phenyl]-amide (135), 5-tert-Butyl-2-(4-fluoro-benzyl)-2H-pyrazole-3-carboxylic acid [4-fluoro-3-(pyrazin-2-yloxymethyl)-phenyl]-amide (136), 1-[4-Fluoro-3-(pyrazin-2-yloxymethyl)-phenyl]-3-[5-(tetrahydro-furan-2-yl)-[1,3,4]thiadiazol-2-yl]-urea (140), 6-Methyl-imidazo[2,1-b]thiazole-5-carboxylic acid [4-fluoro-3-(pyrazin-2-yloxymethyl)-phenyl]-amide (144), 3,5-Di-tert-butyl-N-[4-fluoro-3-(pyrazin-2-yloxymethyl)-phenyl]-benzamide (146), 1-Benzyl-6-oxo-1,6-dihydro-pyridine-3-carboxylic acid [4-fluoro-3-(pyrazin-2-yloxymethyl)-phenyl]-amide (147), 1-[4-Fluoro-3-(pyrazin-2-yloxymethyl)-phenyl]-3-(5-methylsulfanyl-[1,3,4]thiadiazol-2-yl)-urea (149), 2,6-Di-morpholin-4-yl-pyrimidine-4-carboxylic acid [4-fluoro-3-(pyrazin-2-yloxymethyl)-phenyl]-amide (150), N-[4-Fluoro-3-(pyrazin-2-yloxymethyl)-phenyl]-3-(2-methyl-thiazol-4-yl)-benzamide (151), 1-(2-Benzyl-5-tert-butyl-2H-pyrazol-3-yl)-3-[4-fluoro-3-(pyrazin-2-yloxymethyl)-phenyl]-urea (153), 1-(2-Benzothiazol-2-yl-5-tert-butyl-2H-pyrazol-3-yl)-3-[4-fluoro-3-(pyrazin-2-yloxymethyl)-phenyl]-urea (155), 1-[5-tert-Butyl-2-(6-chloro-pyridazin-3-yl)-2H-pyrazol-3-yl]-3-[4-fluoro-3-(pyrazin-2-yloxymethyl)-phenyl]-urea (156), 1-[5-tert-Butyl-2-(2,6-dimethyl-pyrimidin-4-yl)-2H-pyrazol-3-yl]-3-[4-fluoro-3-(pyrazin-2-yloxymethyl)-phenyl]-urea (157), 1-[4-Fluoro-3-(pyrazin-2-yloxymethyl)-phenyl]-3-(5-methanesulfinyl-[1,3,4]thiadiazol-2-yl)-urea (159), 1-(5-tert-Butyl-2-pyridin-4-yl-2H-pyrazol-3-yl)-3-[4-fluoro-3-(pyrazin-2-yloxymethyl)-phenyl]-urea (160), 1-[2-(4-Fluoro-phenyl)-5-(tetrahydro-furan-2-yl)-2H-pyrazol-3-yl]-3-[4-fluoro-3-(pyrazin-2-yloxymethyl)-phenyl]-urea (161), 1-[5-tert-Butyl-2-(4-methanesulfonyl-phenyl)-2H-pyrazol-3-yl]-3-[4-fluoro-3-(pyrazin-2-yloxymethyl)-phenyl]-urea (163), 1-[2-(4-tert-Butyl-phenyl)-5-cyclopropyl-2H-pyrazol-3-yl]-3-[4-fluoro-3-(pyrazin-2-yloxymethyl)-phenyl]-urea (164) and 1-[2-(4-Fluoro-phenyl)-5-(tetrahydro-pyran-4-yl)-2H-pyrazol-3-yl]-3-[4-fluoro-3-(pyrazin-2-yloxymethyl)-phenyl]-urea (165).

Preferred compounds of formula IIb include N-[4-Chloro-3-(pyrazin-2-yloxymethyl)-phenyl]-2-morpholin-4-yl-isonicotinamide (93), N-[4-Chloro-3-(pyrazin-2-yloxymethyl)-phenyl]-3-fluoro-5-morpholin-4-yl-benzamide (94), 3-tert-Butyl-N-[4-fluoro-3-(pyrazin-2-yloxymethyl)-phenyl]-benzamide (103), 1-(5-tert-Butyl-2-phenyl-2H-pyrazol-3-yl)-3-[4-fluoro-3-(pyrazin-2-yloxymethyl)-phenyl]-urea (106), 1-[4-Fluoro-3-(pyrazin-2-yloxymethyl)-phenyl]-3-(5-phenyl-1H-pyrazol-3-yl)-urea (118), 1-[4-Fluoro-3-(pyrazin-2-yloxymethyl)-phenyl]-3-[5-(tetrahydro-furan-2-yl)-[1,3,4]thiadiazol-2-yl]-urea (120), 1-(5-Benzyl-[1,3,4]thiadiazol-2-yl)-3-[4-fluoro-3-(pyrazin-2-yloxymethyl)-phenyl]-urea (121), 1-[5-tert-Butyl-2-(2,4-difluoro-phenyl)-2H-pyrazol-3-yl]-3-[4-fluoro-3-(pyrazin-2-yloxymethyl)-phenyl]-urea (125), 1-[5-tert-Butyl-2-(4-chloro-phenyl)-2H-pyrazol-3-yl]-3-[4-fluoro-3-(pyrazin-2-yloxymethyl)-phenyl]-urea (127), 1-[5-(4-Chloro-phenyl)-2-phenyl-2H-pyrazol-3-yl]-3-[4-fluoro-3-(pyrazin-2-yloxymethyl)-phenyl]-urea (128), 1-(5-tert-Butyl-2-p-tolyl-2H-pyrazol-3-yl)-3-[4-fluoro-3-(pyrazin-2-yloxymethyl)-phenyl]-urea (130), 1-[5-(4-Chloro-phenyl)-2-(4-fluoro-phenyl)-2H-pyrazol-3-yl]-3-[4-fluoro-3-(pyrazin-2-yloxymethyl)-phenyl]-urea (132), 1-(2,5-Diphenyl-2H-pyrazol-3-yl)-3-[4-fluoro-3-(pyrazin-2-yloxymethyl)-phenyl]-urea (134), 1-[4-Fluoro-3-(pyrazin-2-yloxymethyl)-phenyl]-3-[5-(tetrahydro-furan-2-yl)-[1,3,4]thiadiazol-2-yl]-urea (140), 3,5-Di-tert-butyl-N-[4-fluoro-3-(pyrazin-2-yloxymethyl)-phenyl]-benzamide (146), 1-[4-Fluoro-3-(pyrazin-2-yloxymethyl)-phenyl]-3-(5-methylsulfanyl-[1,3,4]thiadiazol-2-yl)-urea (149), N-[4-Fluoro-3-(pyrazin-2-loxymethyl)-phenyl]-3-(2-methyl-thiazol-4-yl)-benzamide (151), 1-(2-Benzyl-5-tert-butyl-2H-pyrazol-3-yl)-3-[4-fluoro-3-(pyrazin-2-yloxymethyl)-phenyl]-urea (153), 1-[5-tert-Butyl-2-(6-chloro-pyridazin-3-yl)-2H-pyrazol-3-yl]-3-[4-fluoro-3-(pyrazin-2-yloxymethyl)-phenyl]-urea (156), 1-(5-tert-Butyl-2-pyridin-4-yl-2H-pyrazol-3-yl)-3-[4-fluoro-3-(pyrazin-2-yloxymethyl)-phenyl]-urea (160), 1-[2-(4-Fluoro-phenyl)-5-(tetrahydro-furan-2-yl) -2H-pyrazol-3-yl]-3-[4-fluoro-3-(pyrazin-2-yloxymethyl)-phenyl]-urea (161), 1-[5-tert-Butyl-2-(4-methanesulfonyl-phenyl)-2H-pyrazol-3-yl]-3-[4-fluoro-3-(pyrazin-2-yloxymethyl)-phenyl]-urea (163) and 1-[2-(4-Fluoro-phenyl)-5-(tetrahydro-pyran-4-yl)-2H-pyrazol-3-yl]-3-[4-fluoro-3-(pyrazin-2-yloxymethyl)-phenyl]-urea (165).

Most preferred are N-[4-Chloro-3-(pyrazin-2-yloxymethyl)-phenyl]-3-fluoro-5-morpholin-4-yl-benzamide (94), 3-tert-Butyl-N-[4-fluoro-3-(pyrazin-2-yloxymethyl)-phenyl]-benzamide (103), 1-(5-tert-Butyl-2-phenyl-2H-pyrazol-3-yl)-3-[4-fluoro-3-(pyrazin-2-yloxymethyl)-phenyl]-urea (106), 1-[4-Fluoro-3-(pyrazin-2-yloxymethyl)-phenyl]-3-(5-phenyl-1H-pyrazol-3-yl)-urea (118), 1-[5-tert-Butyl-2-(2,4-difluoro-phenyl)-2H-pyrazol-3-yl]-3-[4-fluoro-3-(pyrazin-2-yloxymethyl)-phenyl]-urea (125), 1-[5-tert-Butyl-2-(4-chloro-phenyl)-2H-pyrazol-3-yl]-3-[4-fluoro-3-(pyrazin-2-yloxymethyl)-phenyl]-urea (127), 1-[5-(4-Chloro-phenyl)-2-(4-fluoro-phenyl)-2H-pyrazol-3-yl]-3-[4-fluoro-3-(pyrazin-2-yloxymethyl)-phenyl]-urea (132), 1-(2,5-Diphenyl-2H-pyrazol-3-yl)-3-[4-fluoro-3-(pyrazin-2-yloxymethyl)-phenyl]-urea (134), 1-[4-Fluoro-3-(pyrazin-2-yloxymethyl)-phenyl]-3-(5-methylsulfanyl-[1,3,4]thiadiazol-2-yl)-urea (149) and 1-(5-tert-Butyl-2-pyridin-4-yl-2H-pyrazol-3-yl)-3-[4-fluoro-3-(pyrazin-2-yloxymethyl)-phenyl]-urea (160).

Acronyms

For convenience, many chemical moieties are represented using well known abbreviations, including but not limited to, methyl (Me), ethyl (Et), n-propyl (nPr), iso-propyl (iPr), n-butyl (nBu), sec-butyl (sBu), iso-butyl (iBu), tert-butyl (tBu), n-hexyl (nHex), cyclohexyl (cHex), phenyl (Ph), biphenyl (biph), benzyl (Bn), naphthyl (naph), methoxy (MeO), ethoxy (EtO), benzoyl (Bz), and acetyl (Ac).

For convenience, many chemical compounds are represented using well known abbreviations, including but not limited to, methanol (MeOH), ethanol (EtOH), iso-propanol (i-PrOR), methyl ethyl ketone (MEK), ether or diethyl ether (Et₂O), acetic acid (AcOH), dichloromethane (methylene chloride, DCM), acetonitrile (ACN), trifluoroacetic acid (TFA), dimethylformamide (DMF), tetrahydrofuran (THF), and dimethylsulfoxide (DMSO).

Synthesis Routes

Several methods for the chemical synthesis of compounds of the present invention are described herein. These methods may be modified and/or adapted in known ways in order to facilitate the synthesis of additional compounds within the scope of the present invention. The amounts of reactants given are for guidance. Descriptions of general laboratory methods and procedures, useful for the preparation of the compounds of the present invention, are described in Vogel's Textbook of Practical Organic Chemistry (5^{th} edition, Ed. Furniss, B. S., Hannaford, A. J., Smith, P. W. G., Tatchell, A. R., Longmann, UK). Methods for the synthesis of pyridine and pyrazine containing molecules in particular are described in Heterocyclic Chemistry, Joule, J. A., Mills, R., and Smith, G. F., Chapman & Hall, London.

General Routes

The key step in the synthesis of compounds of the present invention is the joining of the pyridine/pyrazine ring to the C₅₋₂₀ aryl group with the intervening —O—CH₂— linkage. As illustrated below, with respect to the pyridine molecule, this is most conveniently achieved by reacting a 3-hydroxy pyridine (or pyrazine) with a halomethyl aryl compound, under basic conditions:

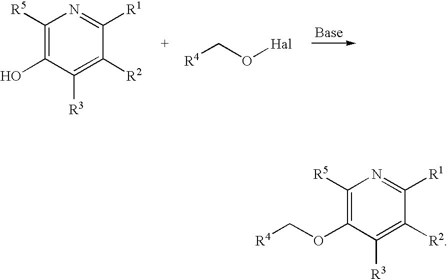

The 3 hydroxy starting material is generally commercially available. The substituents (R¹, R², R³ and R⁵) may be in place in the starting material, having been already introduced using known methods, or may be introduced later in the synthesis, as appropriate. Depending on their structure, protection may be needed to carry out the above step.

The halomethyl aryl compounds may be commercially available or readily synthesised using known techniques. One particular technique for deriving these compounds starts from the corresponding aryl carboxylic acid, which is first reduced, for example, using sodium borohydride, followed by halo-de-halogention, achieved, for example, by the use of triphenyl phosphine.

If the aryl group (R⁴) bears substituents, then these may either be in place at the beginning of the synthesis, or can be added at any appropriate stage. In particular, certain substituents on the aryl group can be modified, using known reactions.
Synthesis of Key Intermediates

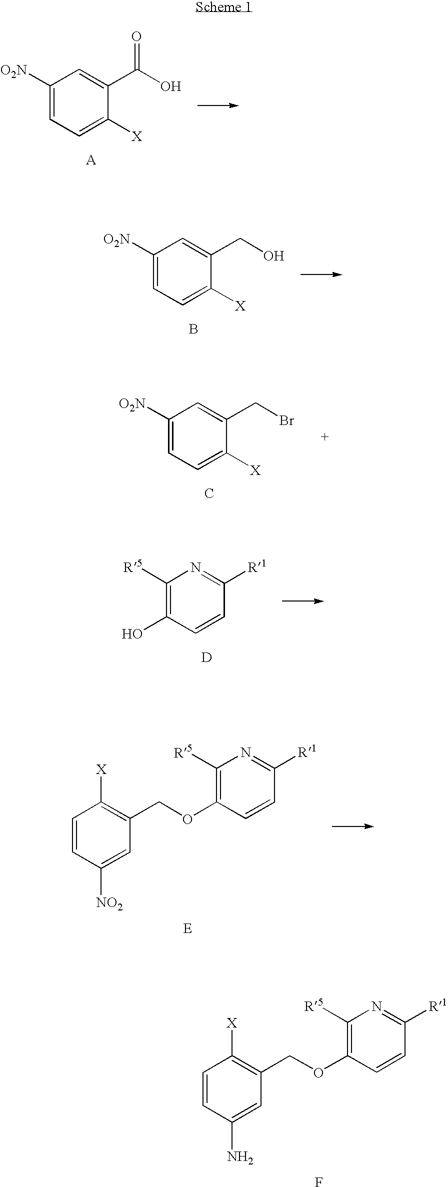

A key intermediate in the synthesis of preferred compounds of the present invention (i.e. those of formula IIa) is the appropriately substituted 3-(pyridin-3-yloxymethyl)-phenylamine (F), as shown in Scheme 1. Scheme 1 illustrates one method of synthesis of this intermediate, although other routes to it are also possible.

The 3-(pyridin-3-yloxymethyl)-phenylamine (F) is synthesised from the corresponding 3-(5-nitro-benzyloxy)pyridine (E) by reduction of the 5-nitro group, using, for example, a metal reducing agent. This 3-(5-nitro-benzyloxy)pyridine (E) is itself synthesised by the base mediated addition of 1-bromomethyl-3-nitro-phenyl (C), or 6-halo equivalent, to the appropriately substituted 3-hydroxy pyridine (D).

The 1-bromomethyl-3-nitro-phenyl (C), or 6-halo equivalent, can be synthesised from the corresponding 3-nitro-benzoic acid (A), via the (3-nitro-phenyl) methanol (B). The first step is a reduction, using, for example, sodium borohydride, and the second step is a halo-de-hydroxylation, achieved, for example, by the use of triphenyl phosphine and carbon tetrabromide.

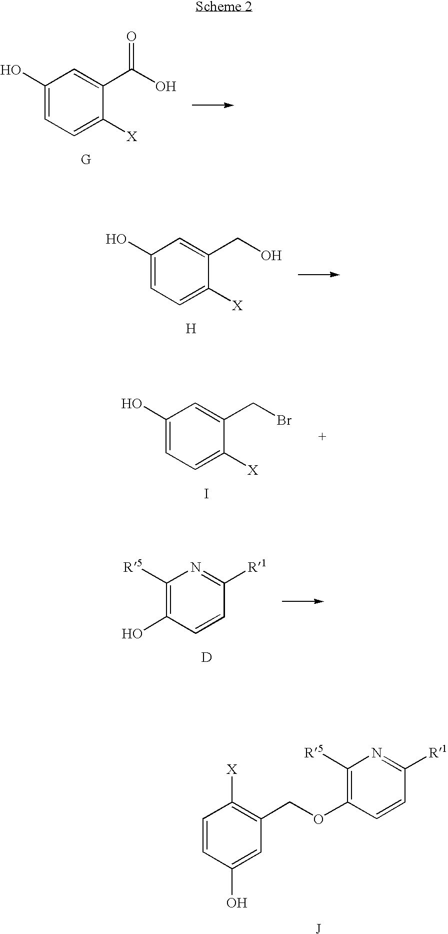

Another key intermediate in the synthesis of preferred compounds of the present invention (of formula IIa) is an appropriately substituted 3-(pyridin-3-yloxymentyl)phenol (J), as shown in Scheme 2. Scheme 2 illustrates one method of synthesis of this intermediate, although other routes to it are possible.

The 3-(pyridin-3-yloxymentyl)phenol (J) is synthesised by the base mediated addition of 1-bromomethyl-3-hydroxy-phenyl (I), or 6-halo equivalent, to the appropriately substituted 3-hydroxy pyridine (D).

The 1-bromomethyl-3-hydroxy-phenyl (I), or 6-halo equivalent, can be synthesised from the corresponding 3-hydroy-benzoic acid (G), via the (3-hydroxy)-phenyl) methanol (H). The first step is a reduction, using, for example sodium borohydride, and the second step is a halo-de-hydroxylation, achieved, for example, by the use of triphenyl phosphine and carbon tetrabromide.

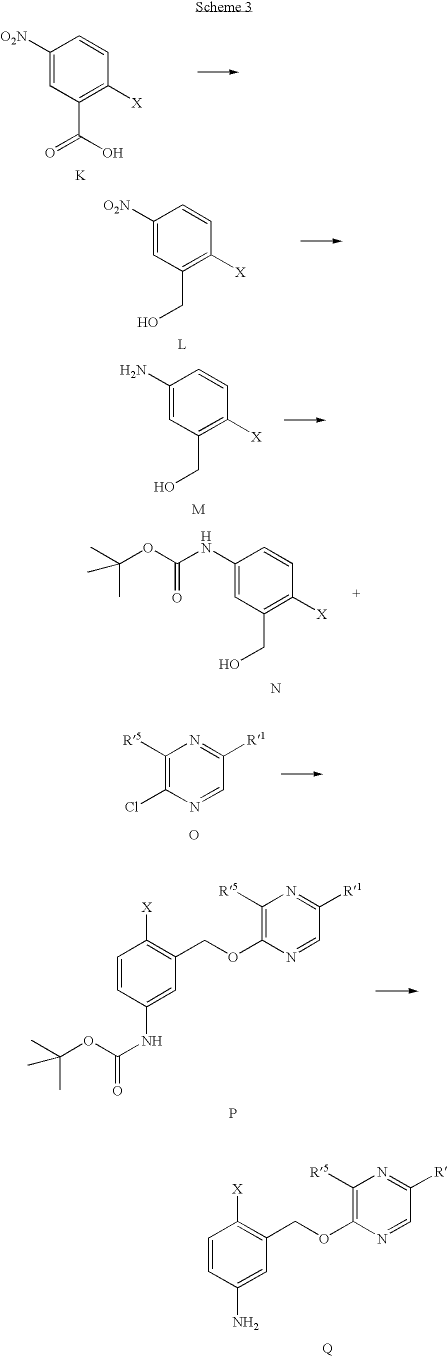

A key intermediate in the synthesis of further preferred compounds of the present invention (i.e. those of formula IIb) is the appropriately substituted 3-(pyrazin-3-yloxymethyl)-phenylamine (Q), as shown in Scheme 3. Scheme 3 illustrates one method of synthesis of this intermediate, although other routes to it are also possible.

The 3-(pyrazin-3-yloxymethyl)-phenylamine (Q) is obtained from the corresponding [3-(pyrazine-3-yloxymethyl)-phenyl] carbamic acid tert-butyl ester (P) by acid mediated deprotection, for example, with a saturate ethyl acetate/HCl solution. The [3-(pyrazine-3-yloxymethyl)-phenyl] carbamic acid tert-butyl ester (P) is synthesised by the base mediated addition of (3-hydroxymethyl-phenyl)-carbamic acid tert-butyl ester (N), or its 4-halo equivalent, to the appropriate 3-chloropyrazine (O).

The (3-hydroxymethyl-phenyl)-carbamic acid tert-butyl ester (N) is a protected version of (5-amino-phenyl) methanol (M), or its 2-halo equivalent, the protecting step being carried out using, for example, di-(tert-butylcarbonyloxy)anhydride (BOC anhydride). The (5-amino-phenyl) methanol (M), or its 2-halo equivalent, is itself obtained by reduction of the corresponding (5-nitro-phenyl) methanol (L), for example by hydrogenation using a palladium catalyst. The (5-nitro-phenyl) methanol (L) can be synthesised from the corresponding 5-nitrobenzoic acid (K) by reduction, using, for example, a boron reducing agent.

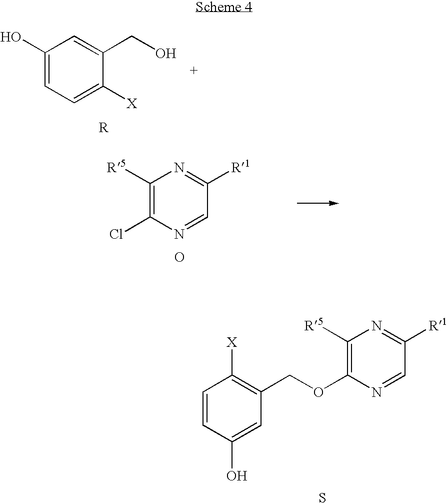

Another key intermediate in the synthesis of preferred compounds of the present invention (of formula IIb) is an appropriately substituted 3-(pyrazin-3-yloxymentyl)phenol (S), as shown in Scheme 4. Scheme 4 illustrates one method of synthesis of this intermediate, although other routes to it are possible.

The 3-(pyrazin-3-yloxymentyl)phenol (S) is synthesised by the base mediated addition of 3-hydroxy benzyl alcohol (R), or 6-halo equivalent, to the appropriately substituted 3-chloro pyrazine (O).

Detailed Routes

R¹

When R¹ is —NRR′, one possible method of introducing this substituent is to synthesise the desired compound with R¹═F, and then carry out direct substitution with HNRR′.

When R¹ is —C(═O)NRR′, the desired product can be synthesised with R¹═—C(═O)OH, followed by addition of HNRR′, using conventional means to aid amide bond formation (see above).

When R¹ is —NHC(═O)NRR′, the desired product can be synthesised with R¹═—C(═O)OH, which can then be converted to —C(═O)—N₃⁻, using, for example thionyl chloride followed by sodium azide, followed by heating to undergo a Curtius rearrangement to the corresponding isocyanate, which then can undergo addition of HNRR′ to form the desired final product.

The isocyanate can also be trapped using tert-butanol to yield a tert-butyl protected carbamic acid, which then undergo base mediated substitution of an appropriate halo-compound (Hal-R), to provide an alternative route to compounds where R¹ is NHR.

When R¹ is —NHSO₂R, the desired product can be synthesised using the methods described in J. Med. Chem., 1991, 34(4), 1356-1362, JP 57-038777 and J. Het. Chem., 1980, 17(1), 11-16.

When R¹ is —NH—C(═O)—R, the desired product can be derived from compounds where R¹═NH₂, by reaction with R—C(═O)OH, or an activated version thereof, for example R—C(═O)C1.

Derivatising R⁴ (Illustrated for R⁴ Phenyl)

The derivatisation routes shown below in schemes 5 to 8, are particularly applicable to the synthesis of compounds of formulae IIa and IIb from the key intermediates above.

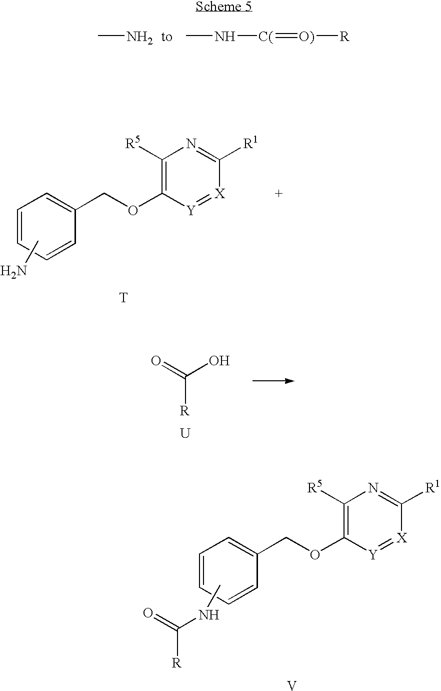

Where it is desired to derivatise —NH₂ to —NH—C(═O)—R, the desired compound (V) is made by the reaction between the appropriate phenylamine (T) and the aromatic acid (U), or formic acid (where R is H). Due to the relative unreactivity of the phenyl amine, this reaction is usually carried out with the aid of an activator or promoter. Activation of the acid can be achieved by converting it into the corresponding acid chloride, for example, by using oxalyl chloride. An alternative method employs amide bond forming promoters, 1[3-(dimethylamino)propyl]-3-ethylcarbodiimide hydrochloride (EDCI) and 7-aza-1-hydroxybenzotriazole (HOAt) or 1-hydroxy benzotriazole (HOBt).

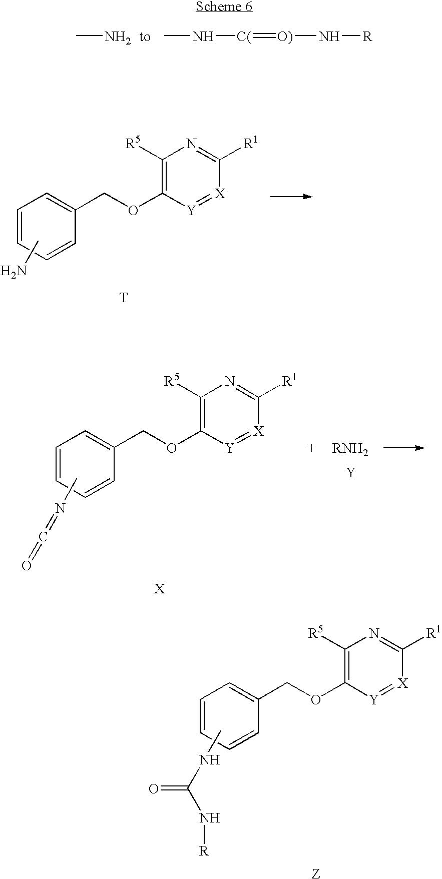

Where it is desired to derivatise —NH₂ to —NH—C(═O)—NH—R, the desired compound (Z) can be synthesised by the conversion of the appropriate phenylamine (T) to the corresponding isocyanate (X), followed by addition of the appropriate aromatic amine (Y), or ammonium hydroxide (where R═H) without the need for isolation of the isocyanate (X).

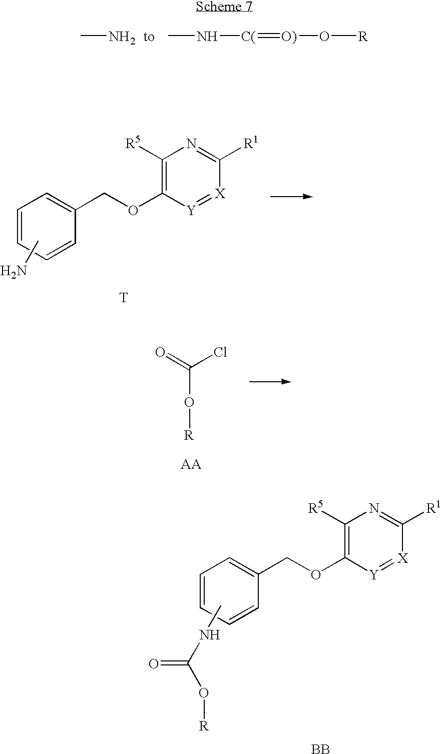

Where it is desired to derivatise —NH₂ to —NH—C(═O)—O—R, the desired compound (BB) can be synthesised by the addition of the appropriate aromatic chloroformate (AA) to the appropriate phenylamine (T).

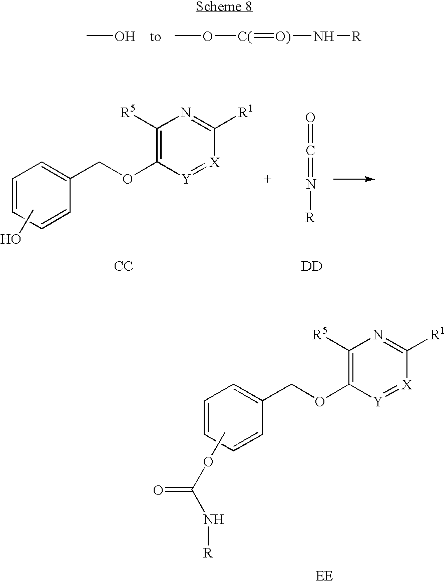

The desired compound (EE) is made by the base mediated reaction between the appropriate phenol (CC) and the aromatic isocynate (DD), or TMS isocyanate (where R is H). An appropriate base would be triethylamine.

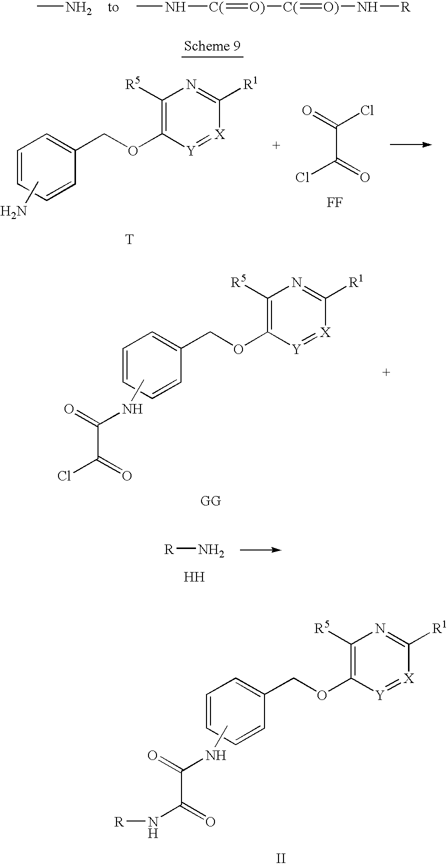

Where it is desired to derivatise —NH₂ to —NH—C(═O)—C(═O)—NH—R, the desired compound (II) is made via the intermediae GG without isolation. The appropriate phenylamine (T) is first reacted with oxalyl chloride, followed by the appropriate amine (HH) to give the desired oxalamide (II).

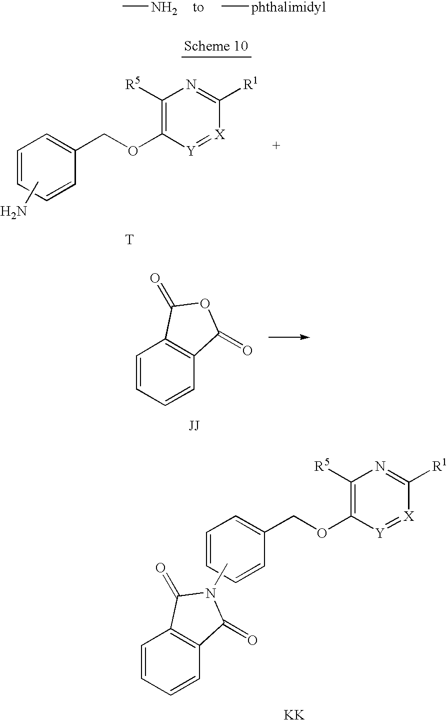

Where it is desired to derivatise —NH₂ to -phthalimidyl, the desired compound (KK) is made by reacting phthalic anhydride (JJ) with the appropriate phenylamine (T).

Protection

In the above routes, groups sensitive to the reaction condition can be appropriately protected to avoid side products being formed. For example, in the routes illustrated above, if one of R¹ to R⁵ is —OH or —SH, and alkylation with an electrophilic reagent onto HX or Q might be expected to also undesirably substitute these groups, protecting groups for —OH and —SH can be employed (see above discussion of protecting groups).

Use of Compounds of the Invention

The present invention provides active compounds, specifically, active pyridine and pyrazine derivatives as defined in the first aspect.

The term “active,” as used herein, pertains to compounds which are capable of inhibiting p38 MAP kinase activity, and specifically includes both compounds with intrinsic activity (drugs) as well as prodrugs of such compounds, which prodrugs may themselves exhibit little or no intrinsic activity.

One of ordinary skill in the art is readily able to determine whether or not a candidate inhibits p38 MAP kinase activity. For example, an assay which may conveniently be used in order to assess the inhibition of p38 MAP kinase activity offered by a particular compound is described in the examples below.

The present invention further provides a method of inhibiting p38 MAP kinase activity in a cell, comprising contacting said cell with an effective amount of an active compound, preferably in the form of a pharmaceutically acceptable composition. Such a method may be practised in vitro or in vivo.

The invention further provides active compounds for use in a method of treatment of the human or animal body. Such a method may comprise administering to such a subject a therapeutically-effective amount of an active compound, preferably in the form of a pharmaceutical composition.

The term “treatment” as used herein in the context of treating a condition, pertains generally to treatment and therapy, whether of a human or an animal (e.g. in veterinary applications), in which some desired therapeutic effect is achieved, for example, the inhibition of the progress of the condition, and includes a reduction in the rate of progress, a halt in the rate of progress, amelioration of the condition, and cure of the condition. Treatment as a prophylactic measure (i.e. prophylaxis) is also included.

The term “therapeutically-effective amount” as used herein, pertains to that amount of an active compound, or a material, composition or dosage from comprising an active compound, which is effective for producing some desired therapeutic effect, commensurate with a reasonable benefit/risk ratio, when administered in accordance with a desired treatment regimen.

The term “treatment” includes combination treatments and therapies, in which two or more treatments or therapies are combined, for example, sequentially or simultaneously. Examples of treatments and therapies include, but are not limited to, chemotherapy (the administration of active agents, including, e.g., drugs, antibodies (e.g., as in immunotherapy), prodrugs (e.g., as in photodynamic therapy, GDEPT, ADEPT, etc.); surgery; radiation therapy; and gene therapy.

The invention further provides the use of an active compound for the manufacture of a medicament, for example, for the treatment of a condition ameliorated by the inhibition of p38 MAP kinase.

The invention further provides a method of treatment of the human or animal body, the method comprising administering to a subject in need of treatment a therapeutically-effective amount of an active compound, preferably in the form of a pharmaceutical composition.

Active compounds may also be used as part of an in vitro assay, for example, in order to determine whether a candidate host is likely to benefit from treatment with the compound in question.

Administration

The active compound or pharmaceutical composition comprising the active compound may be administered to a subject by any convenient route of administration, whether systemically/peripherally or at the site of desired action, including but not limited to, oral (e.g. by ingestion); topical (including e.g. transdermal, intranasal, ocular, buccal, and sublingual); pulmonary (e.g. by inhalation or insufflation therapy using, e.g. an aerosol, e.g. through mouth or nose); rectal; vaginal; parenteral, for example, by injection, including subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, and intrasternal; by implant of a depot, for example, subcutaneously or intramuscularly.

The subject may be a eukaryote, an animal, a vertebrate animal, a mammal, a rodent (e.g. a guinea pig, a hamster, a rat, a mouse), murine (e.g. a mouse), canine (e.g. a dog), feline (e.g. a cat), equine (e.g. a horse), a primate, simian (e.g. a monkey or ape), a monkey (e.g. marmoset, baboon), an ape (e.g. gorilla, chimpanzee, orang-utan, gibbon), or a human.

Formulations

While it is possible for the active compound to be administered alone, it is preferable to present it as a pharmaceutical composition (e.g. formulation) comprising at least one active compound, as defined above, together with one or more pharmaceutically acceptable carriers, adjuvants, excipients, diluents, fillers, buffers, stabilisers, preservatives, lubricants, or other materials well known to those skilled in the art and optionally other therapeutic or prophylactic agents.

Thus, the present invention further provides pharmaceutical compositions, as defined above, and methods of making a pharmaceutical composition comprising admixing at least one active compound, as defined above, together with one or more pharmaceutically acceptable carriers, excipients, buffers, adjuvants, stabilizers, or other materials, as described herein.

The term “pharmaceutically acceptable” as used herein pertains to compounds, materials, compositions, and/or dosage forms which are, within the scope of sound medical judgment, suitable for use in contact with the tissues of a subject (e.g. human) without excessive toxicity, irritation, allergic response, or other problem or complication, commensurate with a reasonable benefit/risk ratio. Each carrier, excipient, etc. must also be “acceptable” in the sense of being compatible with the other ingredients of the formulation.

Suitable carriers, excipients, etc. can be found in standard pharmaceutical texts, for example, Remington's Pharmaceutical Sciences, 18th edition, Mack Publishing Company, Easton, Pa., 1990.

The formulations may conveniently be presented in unit dosage form and may be prepared by any methods well known in the art of pharmacy. Such methods include the step of bringing into association the active compound with the carrier which constitutes one or more accessory ingredients. In general, the formulations are prepared by uniformly and intimately bringing into association the active compound with liquid carriers or finely divided solid carriers or both, and then if necessary shaping the product.

Formulations may be in the form of liquids, solutions, suspensions, emulsions, elixirs, syrups, tablets, losenges, granules, powders, capsules, cachets, pills, ampoules, suppositories, pessaries, ointments, gels, pastes, creams, sprays, mists, foams, lotions, oils, boluses, electuaries, or aerosols.

Formulations suitable for oral administration (e.g. by ingestion) may be presented as discrete units such as capsules, cachets or tablets, each containing a predetermined amount of the active compound; as a powder or granules; as a solution or suspension in an aqueous or non-aqueous liquid; or as an oil-in-water liquid emulsion or a water-in-oil liquid emulsion; as a bolus; as an electuary; or as a paste.

A tablet may be made by conventional means, e.g., compression or moulding, optionally with one or more accessory ingredients. Compressed tablets may be prepared by compressing in a suitable machine the active compound in a free-flowing form such as a powder or granules, optionally mixed with one or more binders (e.g. povidone, gelatin, acacia, sorbitol, tragacanth, hydroxypropylmethyl cellulose); fillers or diluents (e.g. lactose, microcrystalline cellulose, calcium hydrogen phosphate); lubricants (e.g. magnesium stearate, talc, silica); disintegrants (e.g. sodium starch glycolate, cross-linked povidone, cross-linked sodium carboxymethyl cellulose); surface-active or dispersing or wetting agents (e.g. sodium lauryl sulfate); and preservatives (e.g. methyl p-hydroxybenzoate, propyl p-hydroxybenzoate, sorbic acid). Moulded tablets may be made by moulding in a suitable machine a mixture of the powdered compound moistened with an inert liquid diluent. The tablets may optionally be coated or scored and may be formulated so as to provide slow or controlled release of the active compound therein using, for example, hydroxypropylmethyl cellulose in varying proportions to provide the desired release profile. Tablets may optionally be provided with an enteric coating, to provide release in parts of the gut other than the stomach.

Formulations suitable for topical administration (e.g. transdermal, intranasal, ocular, buccal, and sublingual) may be formulated as an ointment, cream, suspension, lotion, powder, solution, past, gel, spray, aerosol, or oil. Alternatively, a formulation may comprise a patch or a dressing such as a bandage or adhesive plaster impregnated with active compounds and optionally one or more excipients or diluents.

Formulations suitable for topical administration in the mouth include losenges comprising the active compound in a flavoured basis, usually sucrose and acacia or tragacanth; pastilles comprising the active compound in an inert basis such as gelatin and glycerin, or sucrose and acacia; and mouthwashes comprising the active compound in a suitable liquid carrier.

Formulations suitable for topical administration to the eye also include eye drops wherein the active compound is dissolved or suspended in a suitable carrier, especially an aqueous solvent for the active compound.

Formulations suitable for nasal administration, wherein the carrier is a solid, include a coarse powder having a particle size, for example, in the range of about 20 to about 500 microns which is administered in the manner in which snuff is taken, i.e. by rapid inhalation through the nasal passage from a container of the powder held close up to the nose. Suitable formulations wherein the carrier is a liquid for administration as, for example, nasal spray, nasal drops, or by aerosol administration by nebuliser, include aqueous or oily solutions of the active compound.

Formulations suitable for administration by inhalation include those presented as an aerosol spray from a pressurised pack, with the use of a suitable propellant, such as dichlorodifluoromethane, trichlorofluoromethane, dichorotetrafluoroethane, carbon dioxide, or other suitable gases.

Formulations suitable for topical administration via the skin include ointments, creams, and emulsions. When formulated in an ointment, the active compound may optionally be employed with either a paraffinic or a water-miscible ointment base. Alternatively, the active compounds may be formulated in a cream with an oil-in-water cream base. If desired, the aqueous phase of the cream base may include, for example, at least about 30% w/w of a polyhydric alcohol, i.e., an alcohol having two or more hydroxyl groups such as propylene glycol, butane-1,3-diol, mannitol, sorbitol, glycerol and polyethylene glycol and mixtures thereof. The topical formulations may desirably include a compound which enhances absorption or penetration of the active compound through the skin or other affected areas. Examples of such dermal penetration enhancers include dimethylsulfoxide and related analogues.

When formulated as a topical emulsion, the oily phase may optionally comprise merely an emulsifier (otherwise known as an emulgent), or it may comprises a mixture of at least one emulsifier with a fat or an oil or with both a fat and an oil. Preferably, a hydrophilic emulsifier is included together with a lipophilic emulsifier which acts as a stabiliser. It is also preferred to include both an oil and a fat. Together, the emulsifier(s) with or without stabiliser(s) make up the so-called emulsifying wax, and the wax together with the oil and/or fat make up the so-called emulsifying ointment base which forms the oily dispersed phase of the cream formulations.

Suitable emulgents and emulsion stabilisers include Tween 60, Span 80, cetostearyl alcohol, myristyl alcohol, glyceryl monostearate and sodium lauryl sulphate. The choice of suitable oils or fats for the formulation is based on achieving the desired cosmetic properties, since the solubility of the active compound in most oils likely to be used in pharmaceutical emulsion formulations may be very low. Thus the cream should preferably be a non-greasy, non-staining and washable product with suitable consistency to avoid leakage from tubes or other containers. Straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters known as Crodamol CAP may be used, the last three being preferred esters. These may be used alone or in combination depending on the properties required.

Alternatively, high melting point lipids such as white soft paraffin and/or liquid paraffin or other mineral oils can be used.

Formulations suitable for rectal administration may be presented as a suppository with a suitable base comprising, for example, cocoa butter or a salicylate.

Formulations suitable for vaginal administration may be presented as pessaries, tampons, creams, gels, pastes, foams or spray formulations containing in addition to the active compound, such carriers as are known in the art to be appropriate.

Formulations suitable for parenteral administration (e.g. by injection, including cutaneous, subcutaneous, intramuscular, intravenous and intradermal), include aqueous and non-aqueous isotonic, pyrogen-free, sterile injection solutions which may contain anti-oxidants, buffers, preservatives, stabilisers, bacteriostats, and solutes which render the formulation isotonic with the blood of the intended recipient; and aqueous and non-aqueous sterile suspensions which may include suspending agents and thickening agents, and liposomes or other microparticulate systems which are designed to target the compound to blood components or one or more organs. Examples of suitable isotonic vehicles for use in such formulations include Sodium Chloride Injection, Ringer's Solution, or Lactated Ringer's Injection. Typically, the concentration of the active compound in the solution is from about 1 ng/ml to about 10 μg/ml, for example from about 10 ng/ml to about 1 μg/ml. The formulations may be presented in unit-dose or multi-dose sealed containers, for example, ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules, and tablets. Formulations may be in the form of liposomes or other microparticulate systems which are designed to target the active compound to blood components or one or more organs.

Dosage

It will be appreciated that appropriate dosages of the active compounds, and compositions comprising the active compounds, can vary from patient to patient. Determining the optimal dosage will generally involve the balancing of the level of therapeutic benefit against any risk or deleterious side effects of the treatments of the present invention. The selected dosage level will depend on a variety of factors including, but not limited to, the activity of the particular compound, the route of administration, the time of administration, the rate of excretion of the compound, the duration of the treatment, other drugs, compounds, and/or materials used in combination, and the age, sex, weight, condition, general health, and prior medical history of the patient. The amount of compound and route of administration will ultimately be at the discretion of the physician, although generally the dosage will be to achieve local concentrations at the site of action which achieve the desired effect without causing substantial harmful or deleterious side-effects.

Administration in vivo can be effected in one dose, continuously or intermittently (e.g. in divided doses at appropriate intervals) throughout the course of treatment. Methods of determining the most effective means and dosage of administration are well known to those of skill in the art and will vary with the formulation used for therapy, the purpose of the therapy, the target cell being treated, and the subject being treated. Single or multiple administrations can be carried out with the dose level and pattern being selected by the treating physician.

In general, a suitable dose of the active compound is in the range of about 100 pg to about 10 mg, more preferably 10 ng to 1 mg, per kilogram body weight of the subject per day. Where the active compound is a salt, an ester, prodrug, or the like, the amount administered is calculated on the basis of the parent compound and so the actual weight to be used is increased proportionately.

### EXAMPLES

### Example 1

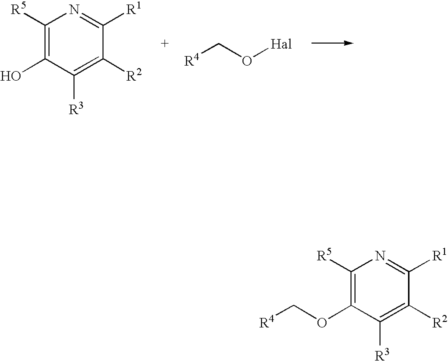

A mixture of the appropriate starting material (a 3 hydroxy pyridine—generally commercially available) (2.00 mmol), the appropriate halo compound (2.20 mmol) and Adogen™ 464 (1 drop) in aqueous 40% NaOH solution (2 ml) and dichloromethane (2 ml) is stirred at room temperature for 19 hours. The dichloromethane is separated and the aqueous layer diluted with water (10 ml) and then extracted with dichloromethane (3×25 ml). The organic extracts are combined, dried (K₂CO₃), filtered and concentrated. Recrystallisation from hexane/dichloromethane or purification using Flash chromatography gives the desired product

From 2-amino-3-hydroxypyridine

2-amino-3-benzyloxypyridine (1): from benzyl chloride; δ_{H} (400 MHz; CDCl₃) 4.70 (2H, br s), 5.07 (2H, s), 6.59 (1H, dd, J 8, 5), 6.96 (1H, dd, J 8, 1.5), 7.40 (5H, m), 7.68 (1H, dd, J 5, 1.5).

2-amino-3-(2-fluorobenzyloxy)pyridine (3): from 2-fluorobenzyl chloride; δ_{H} (400 MHz; CDCl₃) 4.66 (2H, br s), 5.13 (2H, s), 6.61 (1H, dd, J 7.5, 5), 7.01 (1H, dd, J 7.5, 1.5), 7.11 (1H, ddd, J 10, 7.5, 1), 7.17 (1H, td, J 7 .5, 1), 7.34 (1H, m), 7.44 (1H, tm, J 7.5), 7.69 (1H, dd, J 5, 1.5).

2-amino-3-(4-fluorobenzyloxy)pyridine (4): from 4-fluorobenzyl chloride; δ_{H} (400 MHz; CDCl₃) 4.67 (2H, br s), 5.02 (2H, s), 6.59 (1H, dd, J 8, 5), 6.95 (1H, dd, J 8, 1.5), 7.08 (2H, t, J 9), 7.39 (2H, dd, J 9, 5), 7.68 (1H, dd, J 5, 1.5).

2-amino-3-(1-naphthylmethyloxy)pyridine (5): from 1-naphthylmethyl chloride; δ_{H} (400 MHz; CDCl₃) 4.63 (2H, br s), 5.49 (2H, s), 6.64 (1H, dd, J 8, 5), 7.12 (1H, dd, J 8, 1.5), 7.48 (2H, dd, J 8, 7), 7.55 (2H, m), 7.71 (1H, dd, J 5, 1.5), 7.90 (2H, m), 8.03 (1H, m).

2-amino-3-(2-methoxybenzyloxy)pyridine (6): from 2-methoxybenzyl chloride; δ_{H} (400 MHz; CDCl₃) 3.87 (3H, s), 4.70 (2H, br s), 5.11 (2H, s), 6.59 (1H, dd, J 8, 5), 6.93 (1H, d, J 8), 6.99 (2H, m), 7.32 (1H, m) , 7.39 (1H, d, J 7), 7.67 (1H, dd, J 5, 1.5).

2-amino-3-(2-chlorobenzyloxy)pyridine (8): from 2-chlorobenzyl chloride; δ_{H} (400 MHz; CDCl₃) 4.70 (2H, br s), 5.17 (2H, s), 6.59 (1H, dd, J 7.5, 5), 6.96 (1H, dd, J 7.5, 1.5), 7.28 (2H, m), 7.41 (1H, m), 7.47 (1H, m), 7.68 (1H, dd, J 5, 1.5).

2-amino-3-(3-chlorobenzyloxy)pyridine (9): from 3-chlorobenzyl chloride; δ_{H} (400 MHz; CDCl₃) 4.69 (2H, br s), 5.04 (2H, s), 6.59 (1H, dd, J 7.5, 5), 6.93 (1H, dd, J 7.5, 1.5), 7.31 (3H; m), 7.42 (1H, m), 7.69 (1H, dd, J 5, 1.5).

2-amino-3-(2,3-difluorobenzyloxy)pyridine (12): from 2,3-difluorobenzyl chloride; δ_{H} (400 MHz; CDCl₃) 4.67 (2H, br s), 5.14 (2H, s), 6.60 (1H, dd, J 7.5, 5), 6.98 (1H, dd, J 7.5, 1.5), 7.10 (1H, m), 7.15 (1H, m), 7.20 (1H, m), 7.69 (1H, dd, J 5, 1.5).

2-amino-3-(2,4-difluorobenzyloxy)pyridine (13): from 2,4-difluorobenzyl chloride; δ_{H} (400 MHz; CDCl₃) 4.64 (2H, br s), 5.07 (2H, s), 6.60 (1H, dd, J 8, 5), 6.87 (2H, m), 6.98 (1H, dd, J 8, 1.5), 7.41 (1H, td, J 8.5, 6.5), 7.69 (1H, dd, J 5, 1.5).

2-amino-3-(3,4-difluorobenzyloxy)pyridine (14): from 3,4-difluorobenzyl chloride; δ_{H} (400 MHz; CDCl₃) 4.66 (2H, br s), 5.00 (2H, s), 6.58 (1H, dd, J 8, 5), 6.91 (1H, dd, J 8, 1.5), 7.18 (3H, m), 7.69 (1H, dd, J 5, 1.5).

2-amino-3-(2,4-dichlorobenzyloxy)pyridine (15): from 2,4-dichlorobenzyl chloride; δ_{H} (400 MHz; CDCl₃) 4.68 (2H, br s), 5.13 (2H, s), 6.59 (1H, dd, J 8, 5), 6.93 (1H, dd, J 8, 1.5), 7.27 (1H, dd, J 8, 2), 7.40 (1H, d, J 8), 7.43 (1H, d, J 2), 7.69 (1H, dd, J 5, 1.5).

2-amino-3-(4-chloro-3-fluorobenzyloxy)pyridine (16): from 4-chloro-3-fluorobenzyl chloride; δ_{H} (400 MHz; CDCl₃) 4.68 (2H, br s), 5.12 (2H, s), 6.60 (1H, dd, J 8, 5), 6.95 (1H, dd, J 8, 1.5), 7.01 (1H, J td, 8.5, 2.5), 7.17 (1H, dd, J 8.5, 2.5), 7.44 (1H, dd, J 8.5, 6), 7.69 (1H, dd, J 5, 1.5).

2-amino-3-(2-chloro-4,5-(methylenedioxy)benzyloxy)pyridine (18): from 2-chloro-4,5-(methylenedioxy)benzyl chloride; δ_{H} (400 MHz; CDCl₃) 4.67 (2H, br s), 5.06 (2H, s), 5.98 (2H, s), 6.59 (1H, dd, J 8, 5), 6.87 (1H, s), 6.91 (1H, s), 6.94 (1H, dd, J 8, 1.5), 7.68 (1H, dd, J 5, 1.5).

From 3-hydroxypyridine

3-Benzyloxypyridine (7): from benzyl chloride; δ_{H} (400 MHz; CDCl₃) 5.11 (2H, s), 7.21 (1H, ddd, J 8.5, 4.5, 1), 7.25 (1H, ddd, J 8.5, 3, 1.5), 7.39 (5H, m), 8.23 (1H, dd, J 4.5, 1.5), 8.40 (1H, d, J 3).

3-(1-Naphthylmethyloxy)pyridine (11): from 1-naphthylmethyl chloride; δ_{H} (400 MHz; CDCl₃) 5.55 (2H, s), 7.24 (1H, ddd, J 8.5, 4.5, 0.5), 7.34 (1H, ddd, J 8.5, 3, 1.5), 7.54 (4H, m), 7.89 (2H, m), 8.04 (1H, m), 8.26 (1H, dd, J 4.5, 1.5), 8.47 (1H, d, J 3).

From 2-chloro-3-hydroxypyridine

3-Benzyloxy-2-chloropyridine (10): from benzyl chloride; δ_{H} (400 MHz; CDCl₃) 5.19 (2H, s), 7.16 (1H, dd, J 8.0, 4.5), 7.22 (1H, dd, J 8.0, 1.5), 7.32-7.46 (SH, m), 8.00 (1H, dd, J 4.5, 1.5).

The following compounds were made by analogous methods:

2; 17—MS(ES): m/e 229 (M+H); 19; 20—MS(ES): m/e 277 (M+H) 21; 22; 23—MS(ES): m/e 269 (M+H); 25; 26—MS(ES): m/e 279 (M+H); 27; 28; 29; 30—MS(ES): m/e 265 (M+H); 31; 32—MS(ES): m/e 255 (M+H); 33; 34; 35; 36; 37—MS(ES): m/e 242 (M+H); 38; 39—MS(ES): m/e 221 (M+H); 40—MS(ES): m/e 257 (M+H); 41; 42—MS(ES): m/e 250 (M+H); 43—MS(ES): m/e 277 (M+H); 45—MS(ES): m/e 245 (M+H); 46—MS(ES): m/e 521 (M+H); 47—MS(ES): m/e 241 (M+H); 48—MS(ES): m/e 314 (M+H); 51—MS(ES): m/e 360 (M+H); 54—MS(ES): m/e 340 (M+H); 58; 73—MS(ES): m/e 367 (M+H); 74—MS(ES): m/e 342 (M+H); 80—MS(ES): m/e 335 (M+H).

### Example 2

### (a) Synthesis of Key Intermediate: 4-chloro-3-(pyridin-3-yloxymethyl)-phenylamine

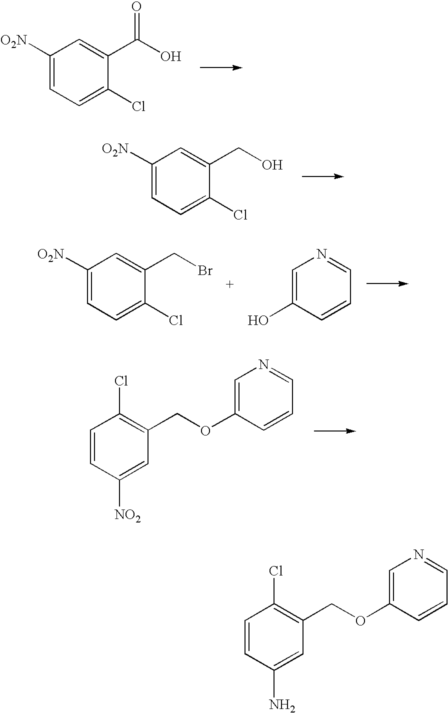 (2-chloro-5-nitro-phenyl)-methanol

To a stirred suspension of sodium borohydride (9.9 mmol) in dry THF (20 ml) at 0° C. was added 2-chloro-5-nitrobenzoic acid (4.96 mmol) dissolved in dry THF (5 ml). Boron trifluoride etherate (13.3 mmol) was added dropwise and the reaction mixture allowed to warm to room temperature over 1 hour. The reaction mixture was quenched with 1N HCl and then partitioned between DCM and water. The organic layer was separated, washed with brine solution, dried (MgSO₄), filtered, evaporated and the residue purified by column chromatography on silica. Elution with mixtures of petroleum ether and ethyl acetate afforded 0.92 g of the desired product; MS(ES): m/e 189 (M+H); δ_{H} (400 MHz, CDCl₃) 8.5 (1H, br s), 8.13 (1H, br dd), 7.54 (1H, d, J 8), 4.89 (2H, s).

2-bromomethyl-1-chloro-4-nitro-benzene

(2-Chloro-5-nitro-phenyl)-methanol (4.9 mmol) was dissolved in DCM (30 ml) and cooled to 0° C. Triphenyl phosphine (5 mmol) was added followed by carbon tetrabromide (4.9 mmol). The reaction mixture was diluted with DCM and washed with water and brine solution. The organic layer was separated, dried (MgSO₄), filtered and evaporated to yield 1.23 g of the desired product; MS (ES): m/e 252 (M+H); δ_{H} (400 MHz, CDCl₃) 8.37 (1H, br s), 8.15 (1H, dd, J 8, 1), 7.61 (1H, d, J 8), 4.63 (2H, s).

3-(2-chloro-5-nitro-benzyloxy)-pyridine

3-Hydroxy pyridine (5.3 mmol) was dissolved in dry DMF (6 ml), cooled to 0° C. and then treated with sodium hydride (60%, 5.5 mmol). After 20 mins, 2-bromomethyl-1-chloro-4-nitro-benzene 4.9 mmol) was added in dry DMF (6 ml) and the reaction mixture stirred at 0° C. for 1 hour. The reaction mixture was quenched with water, then partitioned between ethyl acetate and water. The organic layer was separated, washed with brine solution, dried (MgSO₄), filtered, evaporated and the residue purified by column chromatography on silica. Elution with mixtures of petroleum ether and ethyl acetate afforded 0.32 g of the desired product; MS(ES): m/e 266 (M+H).

4-chloro-3-(pyridin-3-yloxymethyl)-phenylamine

3-(2-chloro-5-nitro-benzyloxy)-pyridine (1.2 mmol) was dissolved in dioxan:water (5:1, 6 ml), and treated with iron powder (10.9 mmol) and iron sulfate heptahydrate (2.66 mmol). The reaction mixture was refluxed for 6 hours, cooled to room temperature and filtered. The filtrate was diluted with ethyl acetate and washed with saturated bicarbonate and brine solution. The organic layer was separated, dried (MgSO₄), filtered and evaporated to give 195 mg of the desired product; MS(ES): m/e 236 (M+H).

The corresponding key intermediates 3-(pyridin-3-yloxymethyl)-phenylamine, 4-fluoro-3-(pyridin-3-yloxymethyl)-phenylamine and 4-chloro-3-(6-hydroxymethylamino-pyridin-3-yloxymethyl)-phenylamine were synthesised in a similar fashion.

### (b) Synthesis of Key Intermediates 4-chloro-3-(6-benzylaminopyridin-3-yloxymethyl)-phenylamine and 4-chloro-3-(2-amino-pyridin-3-yloxymethyl)-phenylamine

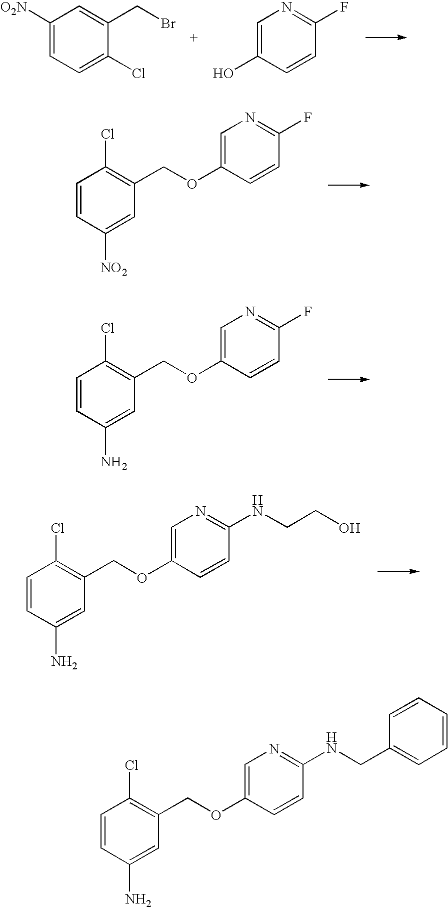 5-(2-Chloro-5-nitro-benzyloxy)-2-fluoro-pyridine

To a solution of 2-fluoro-5-hydroxypyridine (1.77 mmol) in DMF (4 ml) was added NaH (60% dispersion in mineral oil, 4.42 mmol) in small portions at room temperature and under an atmosphere of nitrogen. After stirring for 1 hour, tetra-n-butylammonium chloride (17.68 μmol) was added, followed by 2-chloro-5-nitrobenzyl bromide (5.31 mmol) (see above). After stirring for a further 17 hours, MeOH (2 ml) and then water (2 ml) were added. The DMF was removed in vacuo and the residue was partitioned between ethyl acetate (50 ml) and water (25 ml). The organic layer was separated and the aqueous layer was extracted with ethyl acetate (2×40 ml). The combined organic extracts were then dried (MgSO₄), filtered and concentrated. Purification by flash chromatography eluting with EtOAc/40-60 petroleum ether (1:19) gave the desired compound as a pale yellow oil. δ_{H} (400 MHz; CDCl₃) 5.23 (2H, s), 6.94 (1H, dd, J 8.8 and 3.5), 7.46-7.51 (1H, m), 7.61 (1H, d, J 8.8), 7.95-7.98 (1H, m), 8.19 (1H, dd, J 8.6 and 2.6), 8.49 (1H, d, J 2.6).

4-Chloro-3-(6-fluoro-pyridin-3-yloxymethyl)-phenylamine

To a solution of 5-(2-Chloro-5-nitro-benzyloxy)-2-fluoro-pyridine (5.31 mmol) in dioxane/water (5:1, 30 ml) was added iron powder (47.8 mmol) followed by iron sulphate heptahydrate (11.7 mmol) and the reaction mixture was heated to reflux for a period of 17 hours. Upon cooling, the reaction mixture was filtered through a plug of celite, washed with ethyl acetate (250 ml) and the solvent removed in vacuo. Purification of the residue by flash chromatography eluting with EtOAc/40-60 petroleum ether (3:7) gave the desired compound. δ_{H} (400 MHz; d₆-DMSO) 5.07 (2H, s), 5.33 (2H, br s), 6.55 (1H, dd, J 8.6 and 2.8), 6.74 (1H, d, J 2.8), 7.09 (1H, d, J 8.6), 7.14 (1H, dd, J 9.1 and 3.0), 7.62-7.68 (1H, m), 7.96 (1H, dd, J 3.0 and 1.8).

2-[5-(5-Amino-2-chloro-benzyloxy)-pyridin-2-ylamino]-ethanol

A stirred solution of 4-chloro-3-(6-fluoro-pyridin-3-yloxymethyl)-phenylamine (0.49 mmol) in ethanolamine (2.5 ml) was heated to 130° C. for 24 hours. Upon cooling, the reaction mixture was partitioned between ethyl acetate (80 ml) and water (40 ml). The organic layer was separated and the aqueous layer was extracted with ethyl acetate (2×40 ml). The combined organic extracts were then dried (MgSO₄), filtered and concentrated in vacuo. Purification by flash chromatography eluting with EtOAc/40-60 petroleum ether (1:1) gave the title compound as a pale yellow oil (85 mg, 56%). δ_{H} (400 MHz; CDCl₃) 3.40-3.44 (2H, m), 3.66 (2H, br s), 3.78 (2H, t, J 4.6), 4.66 (1H, br s), 4.99 (2H, s), 6.42 (1H, d, J 8.8), 6.55 (1H, dd, J 8.6 and 2.8), 6.82 (1H, d, J 2.8), 7.12 (1H, d, J 8.6), 7.15 (1H, dd, J 9.0 and 3.0), 7.80 (1H, d, J 2.8).

[5- (5-Amino-2-chloro-benzyloxy)-pyridin-2-yl]-benzylamine

This was prepared in an analogous manner to 2-[5-(5-Amino-2-chloro-benzyloxy)-pyridin-2-ylamino]-ethanol, but using benzylamine in place of ethanolamine. MS(ES): m/e 340 (M+H).

### Example 2(a)

### Synthesis of Compounds Where R⁴ is phenyl-NH—C(═O)—

(a) First Method

Synthesis of N-[4-Chloro-3-pyridin-3-yloxymethyl)-phenyl]-2-morpholin-4-yl-isonicotinamide—44

A stirred solution of 2-morpholin-4-yl-isonicotinic acid (0.24 mmol) in dry DCM (5 ml) at 0° C. was treated with oxalyl chloride (0.29 mmol) and DMF (one drop). The mixture was stirred at 0° C. for 1 hour, then the solvent was removed under reduced pressure. The residue was dissolved in dry DCM (3 ml) and treated dropwise with 4-chloro-3-(pyridin-2-yloxymethyl)-phenylamine (0.16 mmol) and triethylamine (0.16 ml) at 0° C. The reaction mixture was allowed to warm to room temperature overnight, then diluted with DCM and washed with 5% citric acid, saturated bicarbonate solution and brine solution. The organic layer was separated, dried (MgSO₄), filtered, evaporated and the residue purified by column chromatography on silica. Elution with mixtures of petroleum ether and ethyl acetate afforded the desired product. MS(ES): m/e 426 (M+H).

The following compounds were synthesised using a similar method, but with the appropriate starting materials:

From 4-chloro-3-(pyridin-3-yloxymethyl)-phenylamine

N-[4-Chloro-3-(pyridin-3-yloxymethyl)-phenyl3-3-fluoro-5-morpholin-4-yl-benzamide—49, MS(ES): m/e 443 (M+H); N-(4-Chloro-3-(pyridin-3-yloxymethyl)-phenyl]-3-fluoro-benzamide—50, MS(ES): m/e 358 (M+H); N-[4-Chloro-3-(pyridin-3-yloxymethyl)-phenyl]-benzamide—52, MS(ES): m/e 340 (M+H); N-[4-Chloro-3-(pyridin-3-yloxymethyl)-phenyl]-isonicotinamide—53, MS(ES): m/e 341 (M+H); N-[3-(2-Amino-pyridin-3-yloxymethyl)-4-chloro-phenyl]-benzamide—57, MS(ES): m/e 355 (M+H).

From 4-fluoro-3- (pyridin-3-yloxymethyl)-phenylamine

N-[4-Fluoro-3-(pyridin-3-yloxymethyl)-phenyl]-benzamide—59, MS(ES): m/e 323 (M+H); 3-Fluoro-N-[4-fluoro-3-(pyridin-3-yloxymethyl)-phenyl]-benzamide—60, MS(ES): m/e 341 (M+H); 3-Fluoro-N-[4-fluoro-3-(pyridin-3-yloxymethyl)-phenyl]-5-morpholin-4-yl-benzamide—62, MS(ES): m/e 426 (M+H).

From 3-(pyridin-3-yloxymethyl)-phenylamine

N-[3-(Pyridin-3-yloxymethyl)-phenyl]-benzamide—66, MS(ES): m/e 305 (M+H).

(b) Second Method

Synthesis of 3-Tert-butyl-N-[4-chloro-3-(pyridin-3-yloxymethyl)-phenyl]-benzamide—65

A stirred solution 4-chloro-3-(pyridin-2-yloxymethyl)-phenylamine (0.14 mmol) in dry DCM (5 ml) was treated with EDCI (1.68 mmol) and HOAt (1.68 mmol). 3-Tert-butyl benzoic acid (0.14 mmol) was added and the reaction mixture stirred at room temperature overnight. The reaction mixture was diluted with DCM and washed with 5% citric acid, saturated bicarbonate solution and brine solution. The organic layer was separated, dried (MgSO₄), filtered, evaporated and the residue purified by column chromatography on silica. Elution with mixtures of petroleum ether and ethyl acetate afforded the desired product. MS(ES): m/e 396 (M+H)

The following compounds were synthesised using a similar method, but with the appropriate starting materials:

From 4-chloro-3-(6-hydroxymethylamino-pyridin-3-yloxymethyl)-phenylamine

N-{4-Chloro-3-[6-(2-hydroxy-ethylamino)-pyridin-3-yloxymethyl]-phenyl}-3-fluoro-5-morpholin-4-yl-benzamide—76, MS(ES): m/e 502 (M+H).

From 4-chloro-3-(6-benzylamino-pyridin-3-yloxymethyl)-phenylamine

N-[3-(6-Benzylamino-pyridin-3-yloxymethyl)-4-chloro-phenyl]-3-fluoro-5-morpholin-4-yl-benzamide—77, MS(ES): m/e 548 (M+H).

From 4-chloro-3-(pyridin-3-yloxymethyl)-phenylamine

N-[4-Chloro-3-(pyridin-3-yloxymethyl)-phenyl]-3-trifluoromethyl-benzamide—69, MS(ES): m/e 408 (M+H); 3-Chloro-N-[4-chloro-3-(pyridin-3-yloxymethyl)-phenyl]-benzamide—70, MS(ES): m/e 374 (M+H).

From 4-fluoro-3-(pyridin-3-yloxymethyl)-phenylamine

6-Morpholin-4-yl-pyrazine-2-carboxylic acid [4-fluoro-3-(pyridin-3-yloxymethyl)-phenyl]-amide—75, MS(ES): m/e 410 (M+H); 1-(2-tert-Butyl-phenyl)-3-[4-fluoro-3-(pyridin-3-yloxymethyl)-phenyl)-urea—78, MS(ES): m/e 394 (M+H).

From 3-(pyridin-3-yloxymethyl)-phenylamine

3-Fluoro-5-morpholin-4-yl-N-[3-(pyridin-3-yloxymethyl)-phenyl]-benzamide—67, MS(ES): m/e 408 (M+H).

### Example 2(b)

### Synthesis of Compounds Where R⁴ is phenyl-NH—C(═O)—NH—

Synthesis of 1-(5-tert-Butyl-2H-pyrazol-3-yl)-3-[4-chloro-3-(pyridin-3-yloxymethyl)-phenyl]-urea—71

A stirred solution of 4-chloro-3-(pyridin-3-yloxymethyl)-phenylamine (0.21 mmol) in dry DCM (5 ml) at 0° C. was treated with diisopropyl ethylamine (2.13 mmol), followed by triphosgene (0.25 mmol). The mixture was stirred at 0° C. for 3 hours, then treated with 3-amino-5-tert-butyl pyrazole (0.42 mmol). The reaction mixture was allowed to warm to room temperature overnight, then solvent was removed under reduced pressure and the residue partitioned between ethyl acetate and saturated bicarbonate solution. The organic layer was separated, dried (MgSO₄), filtered, evaporated and the residue purified by column chromatography on silica. Elution with mixtures of petroleum ether and ethyl acetate afforded 20 mg of the desired product; MS(ES): m/e 401 (M+H).

The following compounds were synthesised using a similar method, but with the appropriate starting materials:

From 4-chloro-3-(pyridin-3-yloxymethyl)-phenylamine

1-phenyl-3-[4-chloro-3-(pyridin-3-yloxymethyl)-phenyl]-urea—61, MS(ES): m/e 355 (M+H); 1-(5-tert-Butyl-2-phenyl-pyrazol-3-yl)-3-[4-chloro-3-(pyridin-3-yloxymethyl)-phenyl]-urea—64, MS(ES): m/e 477 (M+H); [4-Chloro-3-(pyridin-3-yloxymethyl)-phenyl]-urea, 63, MS(ES): m/e 279 (M+H), using 2M aqueous ammonium chloride in place of aromatic amine.

From 4-fluoro-3-(pyridin-3-yloxymethyl)-phenylamine

1-[4-Fluoro-3-(pyridin-3-yloxymethyl)-phenyl]-3-(5-isopropyl-[1,3,4]thiadiazol-2-yl)-urea—81, MS(ES): m/e 388 (M+H).

### Example 2(c)

### Synthesis of Compounds Where R⁴ is phenyl-NH—C(═O)—O—

Synthesis of [4-Chloro-3-(pyridin-3-yloxymethyl)-phenyl]-carbamic acid phenyl ester—79

A stirred solution of 4-chloro-3-(pyridin-3-yloxymethyl)-phenylamine (0.21 mmol) and pyridine in dry DCM (0.5 ml) at 0° C. was treated with phenyl chloroformate (0.22 mmol). The reaction mixture was warmed to room temperature over 1 hour then diluted with DCM and washed with 5% citric acid, saturated bicarbonate solution and brine solution. The organic layer was separated, dried (MgSO₄), filtered, evaporated and the residue purified by column chromatography on silica. Elution with mixtures of petroleum ether and ethyl acetate afforded 70 mg of the desired product; MS(ES): m/e 356 (M+H).

### Example 2(d)

### Synthesis of Further Compounds Where R⁴ is phenyl-N

Synthesis of N-(4-Chloro-3-(pyridin-3-yloxymethyl)-phenyl]-benzenesulfonamide—55 and N-[4-Chloro-3-(pyridin-3-yloxymethyl)-phenyl]-bisbenzenesulfonamide—56

A stirred solution of 4-chloro-3-(pyridin-3-yloxymethyl)-phenylamine (0.09 mmol) in dry DCM at room temperature was treated with triethylamine (0.18 mmol) and sulfonyl chloride (0.126 mmol). The mixture was stirred at room temperature overnight, then solvent removed under reduced pressure. The residue was diluted with DCM and washed with 5% citric acid, saturated bicarbonate solution and brine solution. The organic layer was separated, dried (MgSO₄), filtered, evaporated and the residue purified by column chromatography on silica. Elution with mixtures of DCM and MeOR afforded the desired products; MS(ES): m/e 376 (M+H) and 516 (M+H).

Synthesis of N-[4-Chloro-3-(pyridin-3-yloxymethyl)-phenyl]-N′-(3-fluoro-5-morpholin-4-yl-phenyl)-oxalamide—72

A stirred solution of 4-chloro-3-(pyridin-3-yloxymethyl)-phenylamine (0.2 mmol) in dry DCM at 0° C. was treated with oxalyl chloride (0.2 mmol). The mixture was stirred at room temperature for 1 hour, then treated with aniline (0.4 mmol) and the reaction mixture stirred overnight at room temperature. The solvent was removed under reduced pressure and the residue was then diluted with ethyl acetate and washed with 5% citric acid, saturated bicarbonate solution and brine solution. The organic layer was separated, dried (MgSO₄), filtered, evaporated and the residue purified by reverse phase HPLC to afford the desired compound; MS(ES): m/e 383 (M+H).

Synthesis of 2-[4-Chloro-3-(pyridin-3-yloxymethyl)-phenyl]-isoindole-1,3-dione—68

A stirred solution of 4-chloro-3-(pyridin-3-yloxymethyl)-phenylamine (0.21 mmol) in dry chloroform at room temperatute was treated with phthalic anhydride (0.91 mmol). The mixture was stirred at room temperature for 1 hour then solvent removed under reduced pressure. The residue was then redissolved in glacial acetic acid and the reaction mixture refluxed overnight. The reaction mixture was then diluted with ethyl acetate and washed with water, saturated bicarbonate solution and brine solution. The organic layer was separated, dried (MgSO₄), filtered, evaporated and the residue purified by column chromatography on silica. Elution with mixtures of petroleum ether and ethyl acetate afforded the title product; MS(ES): m/e 366 (M+H).

### Example 3

### (a) Synthesis of Key Intermediate: 4-fluoro-3-(pyrizin-3-yloxymethyl)-phenylamine

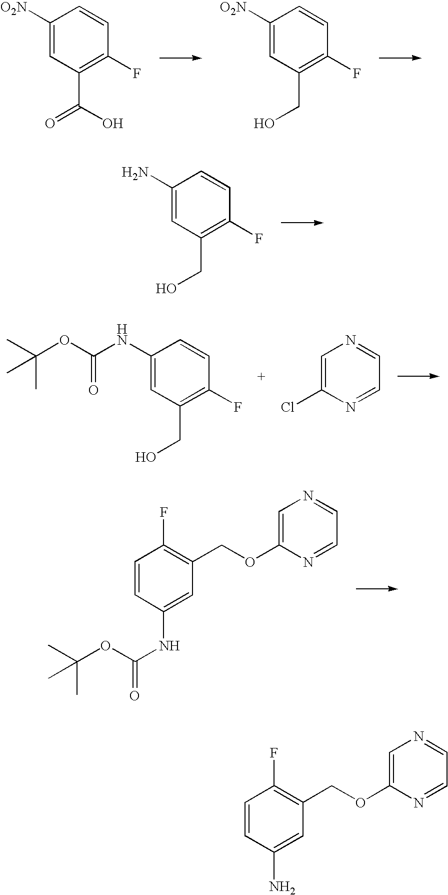 (2-fluoro-5-nitro-phenyl)-methanol

To a stirred suspension of sodium borohydride (44.5 mmol) in dry THF (80 ml) at 0° C. was added 2-fluoro-5-nitrobenzoic acid (2.43 mmol) dissolved in dry THF (50 ml). Boron trifluoride etherate (66.6 mmol) was added dropwise and the reaction mixture allowed to warm to room temperature over 1 hour. The reaction mixture was quenched with 1N HCl and then partitioned between DCM and water. The organic layer was separated, washed with brine solution, dried (MgSO₄), filtered, evaporated and the residue purified by column chromatography on silica. Elution with mixtures of petroleum ether and ethyl acetate afforded the desired product. MS(ES): m/e 172 (M+H).

(5-Amino-2-fluoro-phenyl)-methanol

(2-fluoro-5-nitro-phenyl)-methanol (0.15 mol) was dissolved in ethanol (100 ml), and treated with 10% Pd/C (15 mmol). The reaction mixture was hydrogenated under an atmosphere of hydrogen gas for 6 hours, then the reaction mixture was filtered through celite. The solvent was evaporated to give the desired compound. MS(ES): m/e 142 (M+H).

(4-Fluoro-3-hydroxymethyl-phenyl)-carbamic acid tert-butyl ester

To a stirred solution of (5-Amino-2-fluoro-phenyl)-methanol (12.4 mmol) in dioxan (40 ml) was added di-(tert-butoxycarbonyloxy)anhydride (BOC anhydride) (13.65 mmol) and sodium carbonate (14.89 mmol) in water (40 ml). The reaction mixture was stirred at room temperature overnight, then partitioned between ethyl acetate and water. The organic layer was separated, washed with brine solution, dried (MgSO₄), filtered, evaporated and the residue purified by column chromatography on silica. Elution with mixtures of petroleum ether and ethyl acetate afforded the desired product. MS(ES): m/e 242 (M+H).

[4-Fluor-3-(pyrazin-2-yloxymethyl)-phenyl]-carbamic acid tert-butyl ester

To a stirred solution of (4-Fluoro-3-hydroxymethyl-phenyl)-carbamic acid tert-butyl ester (12.4 mmol) in dry DMF (50 ml) was added sodium hydride (60% dispersion in mineral oil, 25.7 mmol) and the reaction mixture stirred for 30 minutes at room temperature. 2-Chloropyrazine (11.37 mmol) was added and the reaction mixture stirred at room temperature overnight. The reaction mixture was quenched with water and then partitioned between ethyl and water. The organic layer was separated, washed with brine solution, dried (MgSO₄), filtered, evaporated and the residue purified by column chromatography on silica. Elution with mixtures of petroleum ether and ethyl acetate afforded the desired product. MS(ES): m/e 320 (M+H).

4-Fluoro-3-(pyrazin-2-yloxymethyl)-phenylamine

[4-Fluor-3-(pyrazin-2-yloxymethyl)-phenyl]-carbamic acid tert-butyl ester (9.4 mmol) was treated with saturated ethyl acetate/HCl solution (10 ml) at room temperature for 1 hour. The precipitated product was filtered, washed with diethyl ether and dried to afford the desired product. MS(ES): m/e 220 (M+H).

The corresponding key intermediate 4-chloro-3-(pyrazin-2-yloxymethyl)-phenylamine was synthesised in a similar fashion.

### (b) Synthesis of Key Intermediate:3-(pyrazin-2-yloxymethyl)-phenol

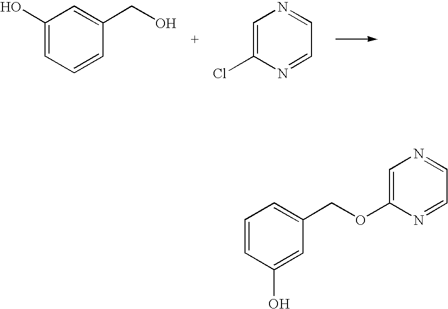

3-Hydroxybenzyl alcohol (8.1 mmol) was dissolved in dry DMF (10 ml), treated with sodium hydride (60% suspension in mineral oil, 9 mmol) and stirred at 0° C. for 30 minutes. 2-Chloropyrazine (8.1 mmol) was added and the reaction mixture stirred at room temperature overnight. The solvent was removed under reduced pressure and the residue was partitioned between ethyl acetate and 1M HCl. The organic layer was separated, washed with saturated sodium bicarbonate solution and brine solution respectively, then dried (MgSO₄), filtered and evaporated to afford the desired product. MS(ES): m/e 203 (M+H).

### Example 3(a)

### Synthesis of Compounds Where R⁴ is phenyl-NH—C(═O)—

Synthesis of N-[4-Fluoro-3-(pyrazin-2-yloxymethyl)-phenyl]-3-phenoxy-benzamide—102

A stirred solution of 4-Fluoro-3-(pyrazin-2-yloxymethyl)-phenylamine (0.46 mmol) in dry DMF (4 ml) was treated with EDCI (0.55 mmol) and HOBt (0.55 mmol). 3-Phenoxy benzoic acid (0.59 mmol) was added and the reaction mixture stirred at room temperature overnight. The solvent was removed under reduced pressure and the residue partitioned between ethyl acetate and water. The organic layer was washed with saturated bicarbonate solution and brine solution, then the organic layer was separated, dried (MgSO₄), filtered and evaporated. The residue was purified by column chromatography on silica, eluting with mixtures of petroleum ether and ethyl acetate to afford the desired product. MS(ES): m/e 416 (M+H).

The following compounds were synthesised using a similar method, but with the appropriate starting materials:

From 4-fluoro-3-(pyrazin-2-yloxymethyl)-phenylamine

N-[4-Fluoro-3-(pyrazin-2-yloxymethyl)-phenyl]-3-(morpholine-4-sulfonyl)-benzamide—98; 4-tert-Butyl-N-[4-fluoro-3-(pyrazin-2-yloxymethyl)-phenyl]-benzamide—101, MS(ES): m/e 380 (M+H);

3-tert-Butyl-N-[4-fluoro-3-(pyrazin-2-yloxymethyl)-phenyl]-benzamide—103, MS(ES): m/e 380 (M+H);

6- (3H-Benzotriazol-1-yloxy)-2-chloro-pyrimidine-4-carboxylic acid [4-fluoro-3-(pyrazin-2-yloxymethyl)-phenyl]-amide—104, MS(ES): m/e 494 (M+H);

2-Chloro-6-methoxy-pyrimidine-4-carboxylic acid [4-fluoro-3-(pyrazin-2-yloxymethyl)-phenyl]-amide—105, MS(ES): m/e 391 (M+H).

3-Methyl-5-phenyl-isoxazole-4-carboxylic acid [4-fluoro-3-(pyrazin-2-yloxymethyl)-phenyl]-amide—122, MS(ES): m/e 405 (M+H);

5-(2-Methyl-thiazol-4-yl)-isoxazole-3-carboxylic acid [4-fluoro-3-(pyrazin-2-yloxymethyl)-phenyl]-amide—124, MS(ES): m/e 412 (M+H);

5-Phenyl-[1,3,4]oxadiazole-2-carboxylic acid [4-fluoro-3-(pyrazin-2-yloxymethyl)-phenyl]-amide—126, MS(ES): m/e 392 (M+H);

Naphthalene-2-carboxylic acid [4-fluoro-3-(pyrazin-2-yloxymethyl)-phenyl]-amide—131, MS(ES): m/e 374 (M+H);

Biphenyl-4-carboxylic acid [4-fluoro-3-(pyrazin-2-yloxymethyl)-phenyl]-amide—133, MS(ES): m/e 400 (M+H);

2-Benzyl-5-tert-butyl-2H-pyrazole-3-carboxylic acid [4-fluoro-3-(pyrazin-2-yloxymethyl)-phenyl]-amide—135, MS(ES): m/e 460 (M+H);

5-tert-Butyl-2-(4-fluoro-benzyl)-2H-pyrazole-3-carboxylic acid [4-fluoro-3-(pyrazin-2-yloxymethyl)-phenyl]-amide—136, MS(ES): m/e 478 (M+H);

6-Methyl-imidazo[2,1-b]thiazole-5-carboxylic acid [4-fluoro-3-(pyrazin-2-yloxymethyl)-phenyl]-amide—144, MS(ES): m/e 384 (M+H);

3,5-Di-tert-butyl-N-[4-fluoro-3-(pyrazin-2-yloxymethyl)-phenyl]-benzamide—146, MS(ES): m/e 436 (M+H);

1-Benzyl-6-oxo-1,6-dihydro-pyridine-3-carboxylic acid [4-fluoro-3-(pyrazin-2-yloxymethyl)-phenyl]-amide—147, MS(ES): m/e 431 (M+H);

2,6-Di-morpholin-4-yl-pyrimidine-4-carboxylic acid [4-fluoro-3-(pyrazin-2-yloxymethyl)-phenyl]-amide—150, MS(ES): m/e 496 (M+H);

N-[4-Fluoro-3-(pyrazin-2-yloxymethyl)-phenyl]-3-(2-methyl-thiazol-4-yl)-benzamide—151, MS(ES): m/e 421 (M+H)

From 4-chloro-3-(pyrazin-2-yloxymethyl)-phenylamine

N-[4-Chloro-3-(pyrazin-2-yloxymethyl)-phenyl]-benzamide—92, MS(ES): m/e 278 (M+H);

N-[4-Chloro-3-(pyrazin-2-yloxymethyl)-phenyl]-2-morpholin-4-yl-isonicotinamide—93, MS(ES): m/e 427 (M+H);

N-[4-Chloro-3-(pyrazin-2-yloxymethyl)-phenyl]-3-fluoro-5-morpholin-4-yl-benzamide, 94, MS(ES): m/e 444 (M+H).

### Example 3(b)

### Synthesis of Compounds Where R⁴ is phenyl-NH—C(═O)—NH—

Synthesis of 1-(5-tert-Butyl-2-phenyl-2H-pyrazol-3-yl)-3-[4-fluoro-3-(pyrazin-2-yloxymethyl)-phenyl]-urea—106

A stirred solution of 4-Fluoro-3-(pyrazin-2-yloxymethyl)-phenylamine (0.39 mmol) in dry DCM (10 ml) at 0° C. was treated with diisopropyl ethylamine (3-9 mmol), followed by triphosgene (0.46 mmol). The mixture was stirred at 0° C. for 3 hours, then treated with 5-tert-butyl-2-phenyl-2H-pyrazol-3-ylamine (0.45 mmol). The reaction mixture was allowed to warm to room temperature overnight, then solvent was removed under reduced pressure and the residue partitioned between ethyl acetate and saturated bicarbonate solution. The organic layer was separated, dried (MgSO₄), filtered, evaporated and the residue purified by column chromatography on silica. Elution with mixtures of petroleum ether and ethyl acetate afforded 20 mg of the desired product. MS(ES): m/e 461 (M+H).

The following compounds were synthesised using a similar method, but with the appropriate starting materials:

From 4-fluoro-3-(pyrazin-2-yloxymethyl)-phenylamine

1-(5-Cyclopropylmethyl-[1,3,4]thiadiazol-2-yl)-3-[4-fluoro-3-(pyrazin-2-yloxymethyl)-phenyl]-urea—96, MS(ES): m/e 401 (M+H);

1-[4-Fluoro-3-(pyrazin-2-yloxymethyl)-phenyl]-3-(5-isopropyl-[1,3,4]thiadiazol-2-yl)-urea—97, MS(ES): m/e 389 (M+H);

1-(4-tert-Butyl-thiazol-2-yl)-3-[4-fluoro-3-(pyrazin-2-yloxymethyl)-phenyl]-urea—100, MS(ES): m/e 402 (M+H);

1-[4-Fluoro-3-(pyrazin-2-yloxymethyl)-phenyl]-3-(5-phenyl-[1,3,4]thiadiazol-2-yl)-urea—115, MS(ES): m/e 423 (M+H);

1-(4,6-Dimethyl-benzothiazol-2-yl)-3-[4-fluoro-3-(pyrazin-2-yloxymethyl)-phenyl]-urea—116, MS(ES): m/e 424 (M+H);

1-[5-(4-Chloro-phenyl)-thiazol-2-yl]-3-[4-fluoro-3-(pyrazin-2-loxymethyl)-phenyl]-urea—117, MS(ES): m/e 457 (M+H);

1-[4-Fluoro-3-(pyrazin-2-yloxymethyl)-phenyl]-3-(5-phenyl-1H-pyrazol-3-yl)-urea—118, MS(ES): m/e 405 (M+H);

1-[4-Fluoro-3-(pyrazin-2-yloxymethyl)-phenyl]-3-(4-phenyl-1H-pyrazol-3-yl)-urea—119, MS(ES): m/e 405 (M+H);

1-[4-Fluoro-3-(pyrazin-2-yloxymethyl)-phenyl]-3-[5-(tetrahydro-furan-2-yl)-[1,3,4]thiadiazol-2-yl]-urea—120, MS(ES): m/e 417 (M+H);

1-(5-Benzyl-[1,3,4]thiadiazol-2-yl)-3-[4-fluoro-3-(pyrazin-2-yloxymethyl)-phenyl]-urea—121, MS(ES): m/e 437 (M+H);

1-[4-Fluoro-3-(pyrazin-2-yloxymethyl)-phenyl]-3-(4-phenyl-thiazol-2-yl)-urea—123, MS(ES): m/e 422 (M+H);

1-[5-tert-Butyl-2-(2,4-difluoro-phenyl)-2H-pyrazol-3-yl]-3-[4-fluoro-3-(pyrazin-2-yloxymethyl)-phenyl]-urea—125, MS(ES): m/e 497 (M+H);

1-[5-tert-Butyl-2-(4-chloro-phenyl)-2H-pyrazol-3-yl]-3-[4-fluoro-3-(pyrazin-2-yloxymethyl)-phenyl]-urea—127, MS(ES): m/e 496 (M+H);

1-[5-(4-Chloro-phenyl)-2-phenyl-2H-pyrazol-3-yl]-3-[4-fluoro-3-(pyrazin-2-yloxymethyl)-phenyl]-urea—128, MS(ES): m/e 516 (M+H);

1-(5-tert-Butyl-2-p-tolyl-2H-pyrazol-3-yl)-3-[4-fluoro-3-(pyrazin-2-yloxymethyl)-phenyl]-urea—130, MS(ES): m/e 475 (M+H);

1-[5-(4-Chloro-phenyl)-2-(4-fluoro-phenyl)-2H-pyrazol-3-yl]-3-[4-fluoro-3-(pyrazin-2-yloxymethyl)-phenyl]-urea—132, MS(ES): m/e 534 (M+H);

1-(2,5-Diphenyl-2H-pyrazol-3-yl)-3-[4-fluoro-3-(pyrazin-2-yloxymethyl)-phenyl]-urea—134, MS(ES): m/e 481 (M+H);

1-[4-Fluoro-3-(pyrazin-2-yloxymethyl)-phenyl]-3-[5-(tetrahydro-furan-2-yl)-[1,3,4]thiadiazol-2-yl]-urea—140, MS(ES): m/e 434 (M+H);

1-[4-Fluoro-3-(pyrazin-2-yloxymethyl)-phenyl]-3-(5-methylsulfanyl-[1,3,4]thiadiazol-2-yl)-urea—149, MS(ES): m/e 393 (M+H);

1-(2-Benzyl-5-tert-butyl-2H-pyrazol-3-yl)-3-[4-fluoro-3-(pyrazin-2-yloxymethyl)-phenyl]-urea—153, MS(ES): m/e 475 (M+H);

1-(2-Benzothiazol-2-yl-5-tert-butyl-2H-pyrazol-3-yl)-3-[4-fluoro-3-(pyrazin-2-yloxymethyl)-phenyl]-urea—155, MS(ES): m/e 519 (M+H);

1-[5-tert-Butyl-2-(6-chloro-pyridazin-3-yl)-2H-pyrazol-3-yl]-3-[4-fluoro-3-(pyrazin-2-yloxymethyl)-phenyl]-urea—156, MS(ES): m/e 498 (M+H);

1-[5-tert-Butyl-2-(2,6-dimethyl-pyrimidin-4-yl)-2H-pyrazol-3-yl]-3-[4-fluoro-3-(pyrazin-2-yloxymethyl)-phenyl]-urea—157, MS(ES): m/e 491 (M+H);

1-[4-Fluoro-3-(pyrazin-2-yloxymethyl)-phenyl]-3-(5-methanesulfinyl-[1,3,4]thiadiazol-2-yl)-urea—159, MS(ES): m/e 409 (M+H);

1-(5-tert-Butyl-2-pyridin-4-yl-2H-pyrazol-3-yl)-3-[4-fluoro-3-(pyrazin-2-yloxymethyl)-phenyl]-urea—160, MS(ES): m/e 462 (M+H);

1-[2-(4-Fluoro-phenyl)-5-(tetrahydro-furan-2-yl)-2H-pyrazol-3-yl]-3-[4-fluoro-3-(pyrazin-2-yloxymethyl)-phenyl]-urea—161, MS(ES): m/e 493 (M+H);

1-[5-tert-Butyl-2-(4-methanesulfonyl-phenyl)-2H-pyrazol-3-yl]-3-[4-fluoro-3-(pyrazin-2-yloxymethyl)-phenyl]-urea—163, MS(ES): m/e 540 (M+H);

1-[2-(4-tert-Butyl-phenyl)-5-cyclopropyl-2H-pyrazol-3-yl]-3-[4-fluoro-3-(pyrazin-2-yloxymethyl)-phenyl]-urea—164, MS(ES): m/e 501 (M+H);

1-[2-(4-Fluoro-phenyl)-5-(tetrahydro-pyran-4-yl)-2H-pyrazol-3-yl]-3-[4-fluoro-3-(pyrazin-2-yloxymethyl)-phenyl]-urea—165, MS(ES): m/e 507 (M+H).

### Example 3(c)

### Synthesis of Compounds Where R⁴ is phenyl-NH—C(═O)—O—

Synthesis of [4-Fluoro-3-(pyrazin-2-yloxymethyl)-phenyl]-carbamic acid 3-trifluoromethyl-phenyl ester—99

A stirred solution of 4-Fluoro-3-(pyrazin-2-yloxymethyl)-phenylamine (0.21 mmol) and pyridine (0.025 ml) in DCM (1 ml) at 0° C. was treated with 3-trifluoromethyl phenyl chloroformate (0.22 mmol) in DCM (0.2 ml). The mixture was warmed to room temperature over 1 hour, then diluted with DCM, washed with 5% citric acid solution, saturated sodium hydrogen carbonate solution and brine solution. Dried (MgSO₄), filtered, evaporated and the residue purified by column chromatography on silica, eluting with mixtures of petroleum ether and ethyl acetate to afford the desired product. MS(ES): m/e 408 (M+H).

### Example 3(d)

### Synthesis of Compounds Where R⁴ is phenyl-O—C(═O)—NH—

Synthesis of Phenyl-carbamic acid 3-(pyrazin-2-yloxymethyl)-phenyl ester—107

A stirred solution 3-(pyrazin-2-yloxymethyl)-phenol (0.49 mmol) in diethyl ether (10 ml) at room temperature was treated with phenylisocyanate (0.49 mmol) and triethylamine (4 drops). The mixture was stirred at room temperature overnight, then the solid precipitate was filtered off, washed with cold ether and dried. The solid was purified by column chromatography on silica. Elution with mixtures of petroleum ether and ethyl acetate afforded the desired product. MS(ES): m/e 322 (M+H).

### Example 3(e)

### Synthesis of Compounds Where R⁴ is dichlorophenyl and R¹ is C(═O)N

Synthesis of 5-(2,6-dichloro-benzyloxy)-pyrazine-2-carboxylic acid (2-sulfamoyl-ethyl)-amide—138

A stirred solution of 5-(2,6-dichloro-benzyloxy)-pyrazine-2-carboxylic acid (0.37 mmol) in dry DMF (5 ml) at room temperature was treated with triethylamine (0.9 mmol), EDCI (0.45 mmol) and HOBt (0.45 mmol). The mixture was stirred at room temperature for 30 mins, then treated with 2-amino-ethanesulfonic acid amide HCl salt (0.45 mmol). The reaction mixture was allowed to warm to room temperature overnight, then solvent was removed under reduced pressure and the residue partitioned between ethyl acetate and water. The organic layer was separated, dried (MgSO₄), filtered, evaporated and the residue purified by column chromatography on silica. Elution with mixtures of petroleum ether and ethyl acetate afforded the desired product. MS(ES): m/e 406 (M+H).

The starting material 5-(2,6-dichloro-benzyloxy)-pyrazine-2-carboxylic acid was prepared as follows:

(i) Lithium-5-chloro-pyrazine-2-carboxylate

5-Chloro-pyrazine-2-carboxylic acid methyl ester (2.9 mmol) was dissolved in tetrahydrofuran/water (5:1, 10 ml), treated with lithium hydroxide (3.04 mmol) and stirred at room temperature overnight. The solvent was removed under reduced pressure to give the desired compound. δ_{H} (400 MHz, CDCl3) 8.5 (1H, br s), 8.13 (1H, br dd), 7.54 (1H, d, J 8), 4.89 (2H, s).

(ii) 5-(2,6-Dichloro-benzyloxy)-pyrazine-2-carboxylic acid

2,6-Dichlorobenzyl alcohol (1.1 mmol) was dissolved in dry DMF (5 ml) and treated with sodium hydride (60% dispersion in mineral oil, 1.21 mmol). The mixture was stirred at room temperature for 30 mins, then treated with lithium-5-chloro-pyrazine-2-carboxylate (1 mmol) and stirred at reflux overnight. The reaction mixture was partitioned between ethyl acetate and water, then the organic layer was separated, dried (MgSO₄), filtered, evaporated and the residue purified by column chromatography on silica. Elution with mixtures of petroleum ether and diethyl ether afforded the title product. MS(ES): m/e 300 (M+H).

The following compounds were synthesised using a similar method, but with the appropriate starting materials:

5-(2,6-Dichloro-benzyloxy)-pyrazine-2-carboxylic acid ethylamide—111;

5-(2,6-Dichloro-benzyloxy)-pyrazine-2-carboxylic acid (2-hydroxy-ethyl)-amide—112;

5-(2,6-Dichloro-benzyloxy)-pyrazine-2-carboxylic acid (2-hydroxy-1,1-dimethyl-ethyl)-amide—113;

5-(2,6-Dichloro-benzyloxy)-pyrazine-2-carboxylic acid (2-hydroxy-1-hydroxymethyl-1-methyl-ethyl)-amide—137;

5-(2,6-Dichloro-benzyloxy)-pyrazine-2-carboxylic acid (1,1-dimethyl-2-pyridin-4-yl-ethyl)-amide—139.

### Example 3(f)

### Synthesis of Compounds Where R⁴ is dichlorophenyl and R¹ is NH

Synthesis of 2-[5-(2,6-Dichloro-benzyloxy)-pyrazin-2-ylamino]-ethanol—158

A stirred solution of [5-(2,6-dichloro-benzyloxy)-pyrazin-2-yl]carbamic acid tert-butyl ester (0.27 mmol) in dry DMF (5 ml) at room temperature was treated with sodium hydride (60% dispersion in mineral oil, 0.35 mmol). The mixture was stirred at room temperature for 30 mins, treated with (2-bromo-ethoxy)-trimethyl-silane (0.32 mmol) and allowed to reflux overnight. The reaction mixture was partitioned between ethyl acetate and water, the organic layer separated, dried (MgSO₄), filtered and evaporated to dryness. The residue was then taken up in dry DCM (5 ml), treated with TFA (0.5 ml) and stirred at room temperature overnight. The solvent was removed under reduced pressure and the residue purified by column chromatography on silica. Elution with mixtures of petroleum ether and ethyl acetate afforded the title product. MS(ES): m/e 315 (M+H).

The starting material [5-(2,6-dichloro-benzyloxy)-pyrazin-2-yl]carbamic acid tert-butyl ester was prepared as follows:

(i) 5-(2,6-Dichloro-benzyloxy)-pyrazine-2-carbonyl azide

5-(2,6-Dichloro-benzyloxy)-pyrazine-2-carboxylic acid (14 mmol) was dissolved in thionyl chloride (30 ml) and heated at reflux for 2 hours. The thionyl chloride was removed under reduced pressure with toluene, the residue dissolved in acetone (60 ml), treated with sodium azide (16.9 mmol) and then stirred overnight at room temperature. The reaction mixture was diluted with water and the solvent removed under reduced pressure. The residue was partitioned between DCM and water then the organic layer was separated, dried (MgSO₄), filtered and evaporated to afford the title product. MS(ES): m/e 325 (M+H)

(ii) [5-(2,6-Dichloro-benzyloxy)-pyrazin-2-yl]carbamic acid tert-butyl ester

5-(2,6-Dichloro-benzyloxy)-pyrazine-2-carbonyl azide (0.31 mmol) was dissolved in toluene (2 ml) and treated with t-butanol (0.6 mmol). The mixture was heated to 100° C. in a sealed tube for 15 mins, then solvent removed under reduced pressure. The residue was purified by column chromatography on silica, eluting with mixtures of petroleum ether and ethyl acetate to give the title product. MS(ES): m/e 371 (M+H).

The following compounds were synthesised using a similar method, but with the appropriate starting materials:

Benzyl-[5-(2,6-dichloro-benzyloxy)-pyrazin-2-yl]-amine—141;

[5-(2,6-Dichloro-benzyloxy)-pyrazin-2-yl]-methyl-amine—148;

4-[5-(2,6-Dichloro-benzyloxy)-pyrazin-2-yl]-morpholine—152;

[5-(2,6-Dichloro-benzyloxy)-pyrazin-2-yl]-(1-phenyl-ethyl)-amine—154.

The following compounds were made by analogous methods to those described above:

82—MS(ES): m/e 252 (M+H); 83—MS(ES): m/e 330 (M+H); 84; 85—MS(ES): m/e 236 (M+H); 86—MS(ES): m/e 202 (M+H); 87—MS(ES): m/e 255 (M+H); 88; 89—MS(ES): m/e 336 (M+H); 90—MS(ES): m/e 270 (M+H); 91—MS(ES): m/e 236 (M+H); 95—MS(ES): m/e 401 (M+H); 108—MS(ES): m/e 311 (M+H); 109—MS(ES): m/e 337 (M+H); 110—MS(ES): m/e 270 (M+H); 114—MS(ES): m/e 369 (M+H); 129—MS(ES): m/e 461 (M+H); 142—MS(ES): m/e 444 (M+H); 143—MS(ES): m/e 433 (M+H); 145; 162—MS(ES): m/e 409 (M+H); 166—MS(ES): m/e 354 (M+H); 167—MS(ES): m/e 355 (M+H); 168—MS(ES): m/e 353 (M+H); 169—MS(ES): m/e 410 (M+H); 170—MS(ES): m/e 410 (M+H); 171—MS(ES): m/e 398 (M+H); 172—MS(ES): m/e 396 (M+H); 173—MS(ES): m/e 397 (M+H); 174—MS(ES): m/e 379 (M+H); 175—MS(ES): m/e 384 (M+H); 176—MS(ES): m/e 386 (M+H); 177—MS(ES): m/e 371 (M+H).

p38 MAP Kinase Assay

In 1 ml of fresh assay buffer (25 mM HEPES pH 7.4, 25 mM β-glycerphosphate, 5 mM EDTA, 15 mM MgCl₂, 100 μM ATP, 1 mM sodium orthovanadate, 1 mM DTT), 35 μg of inactive purified p38 and 0.12 μg of active MKK6 (1688 U/mg—Upstate Biotechnology) are mixed and incubated at room temperature overnight to activate the p38.

The activated p38 is then diluted 1:6 with assay buffer, and 20 μl mixed with 25 μl of MBP mix (300 μl 10×strength assay buffer, 300 μl of 10 mM DTT & 10 mM sodium orthovanadate, 1.7 ml H₂O, 50 μCi γ³³P-ATP, 200 μl of myelin basic protein (MBP) (5 mg/ml)) and added to 96 well plates along with 5 μl of various dilutions of the test compound in DMSO (up to 10%). The reaction is allowed to proceed for 50 minutes before being stopped with an excess of ortho-phosphoric acid (30 μl at 2%).

γ³³P-ATP which remains unincorporated into the myelin basic protein is separated from phosphorylated MBP on a Millipore MAPH filter plate. The wells of the MAPH plate are wetted with 0.5% orthophosphoric acid, and then the results of the reaction are filtered with a Millipore vacuum filtration unit through the wells. Following filtration, the residue is washed twice with 200 μl of 0.5% orthophosphoric acid. Once the filters have dried, 25 μl of Microscint 20 scintillant is added, and then counted on a Packard Topcount for 30 seconds.

The % inhibition of the p38 activity is calculated and plotted in order to determine the concentration of test compound required to inhibit 50% of the p38 activity (IC₅₀).

TABLE 1

| Compound | Structure | IC₅₀ (μM) |
|---|---|---|
| 1 | 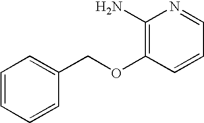 | <2000 |
| 2 | 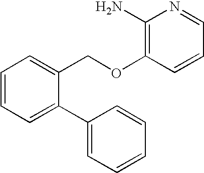 | <10 |
| 3 | 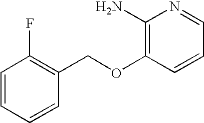 | <1000 |
| 4 | 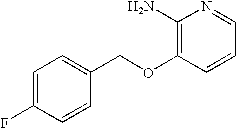 | <2000 |
| 5 | 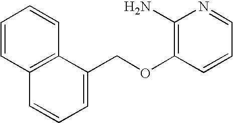 | <200 |
| 6 | 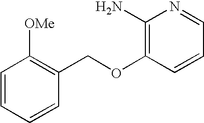 | <1000 |
| 7 | 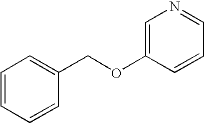 | <2000 |
| 8 | 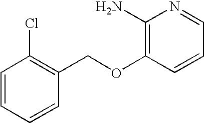 | <1000 |
| 9 | 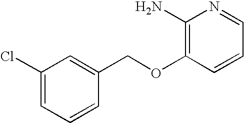 | <1000 |
| 10 | 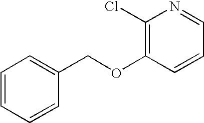 | <2000 |
| 11 | 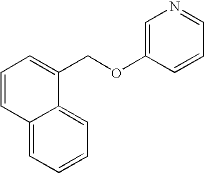 | <1000 |
| 12 | 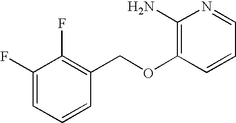 | <1000 |
| 13 | 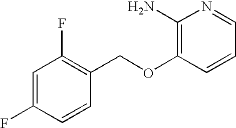 | <1000 |
| 14 | 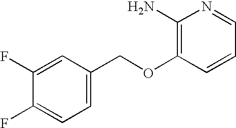 | <1000 |
| 15 | 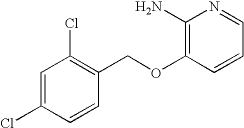 | <1000 |
| 16 | 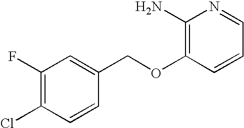 | <200 |
| 17 | 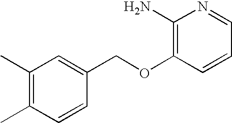 | <1000 |
| 18 | 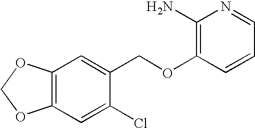 | <1000 |
| 19 | 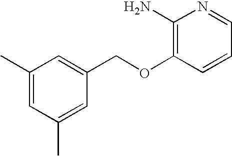 | <200 |
| 20 | 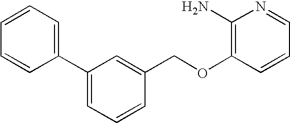 | <200 |
| 21 | 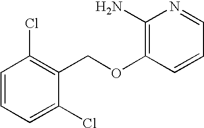 | <1000 |
| 22 | 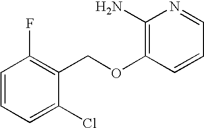 | <1000 |
| 23 | 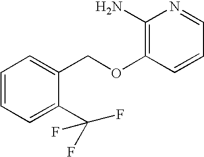 | <100 |
| 24 | 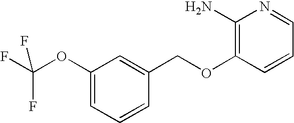 | <10000 |
| 25 | 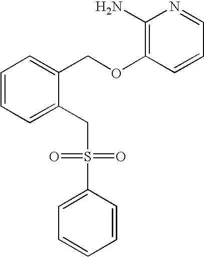 | <1000 |
| 26 | 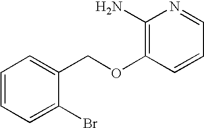 | <1000 |
| 27 | 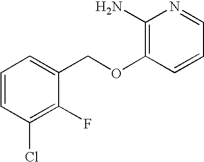 | <1000 |
| 28 | 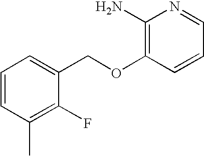 | <1000 |
| 29 | 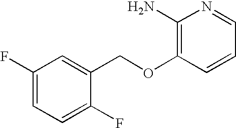 | <1000 |
| 30 | 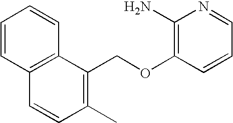 | <100 |
| 31 | 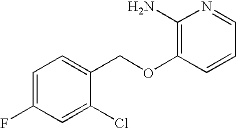 | <1000 |
| 32 | 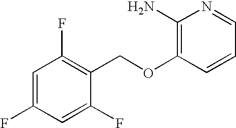 | <1000 |
| 33 | 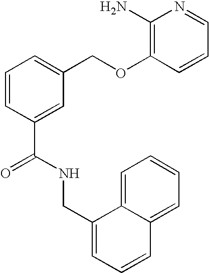 | <1000 |
| 34 | 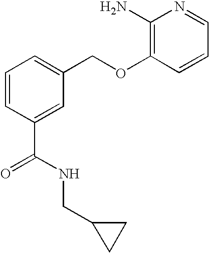 | <1000 |
| 35 | 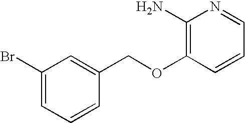 | <1000 |
| 36 | 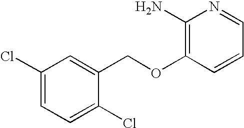 | <1000 |
| 37 | 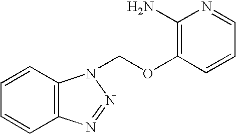 | <1000 |
| 38 | 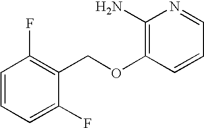 | <1000 |
| 39 | 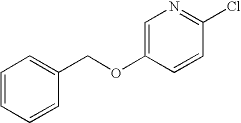 | <1000 |
| 40 | 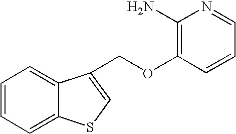 | <1000 |
| 41 | 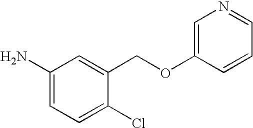 | <1000 |
| 42 | 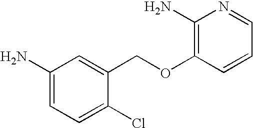 | <1000 |
| 43 | 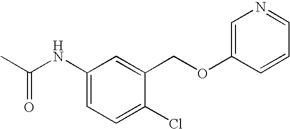 | <1000 |
| 44 | 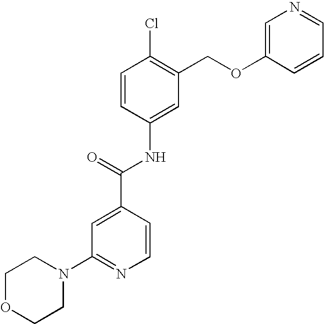 | <2 |
| 45 | 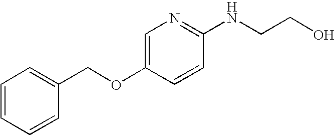 | <200 |
| 46 | 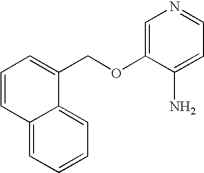 | <1000 |
| 47 | 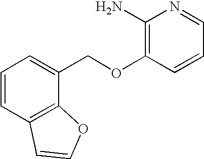 | <1000 |
| 48 | 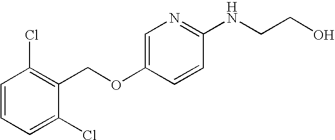 | <200 |
| 49 | 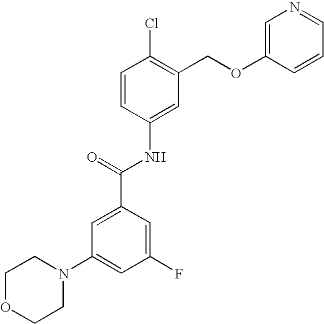 | <2 |
| 50 | 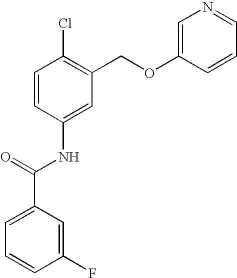 | <200 |
| 51 | 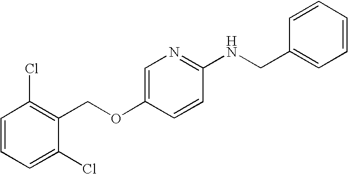 | <200 |
| 52 | 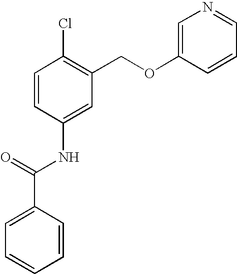 | <20 |
| 53 | 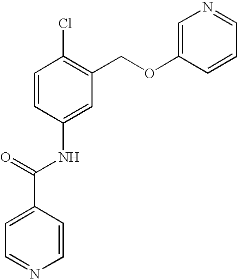 | <200 |
| 54 | 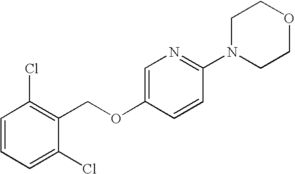 | <1000 |
| 55 | 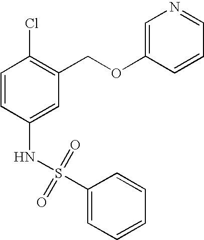 | <200 |
| 56 | 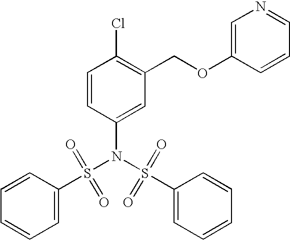 | <1000 |
| 57 | 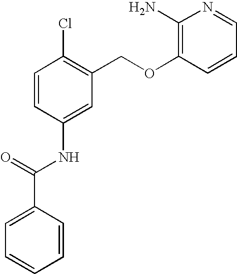 | <20 |
| 58 | 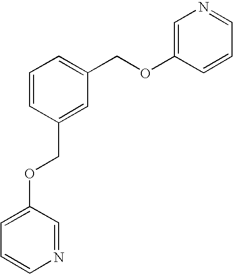 | <1000 |
| 59 | 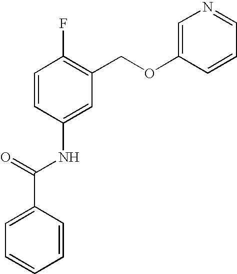 | <200 |
| 60 | 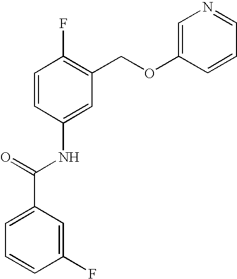 | <200 |
| 61 | 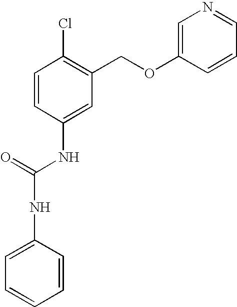 | <20 |
| 62 | 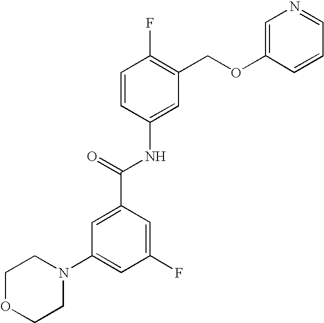 | <2 |
| 63 | 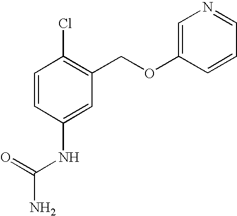 | <20 |
| 64 | 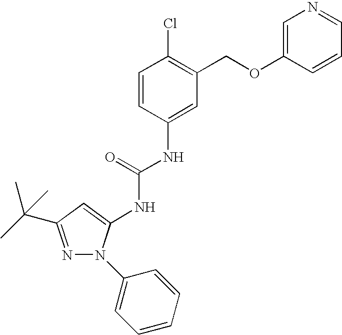 | <2 |
| 65 | 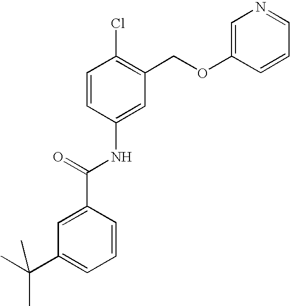 | <2 |
| 66 | 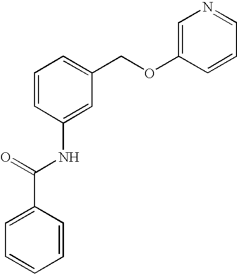 | <200 |
| 67 | 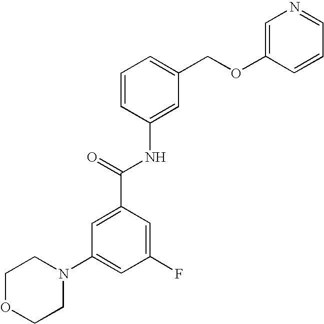 | <20 |
| 68 | 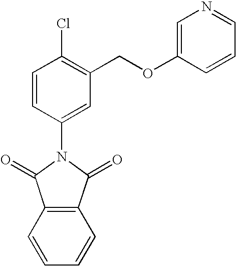 | <1000 |
| 69 | 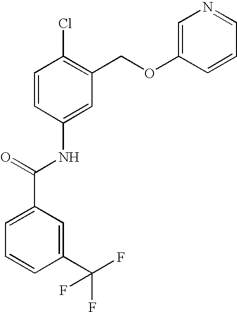 | <20 |
| 70 | 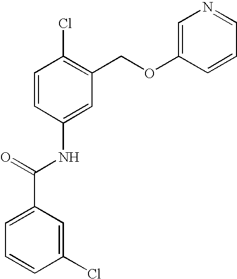 | <20 |
| 71 | 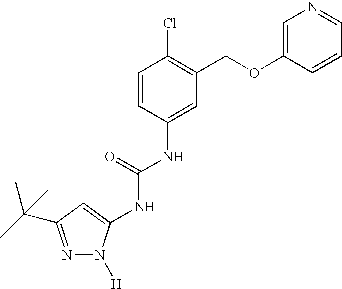 | <2 |
| 72 | 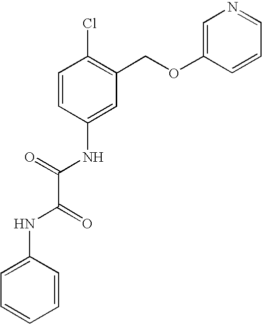 | <2 |
| 73 | 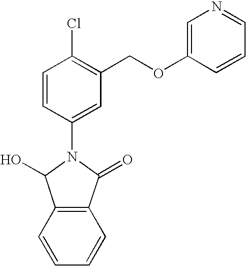 | <200 |
| 74 | 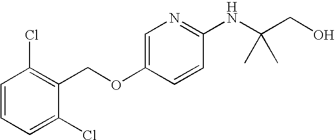 | <200 |
| 75 | 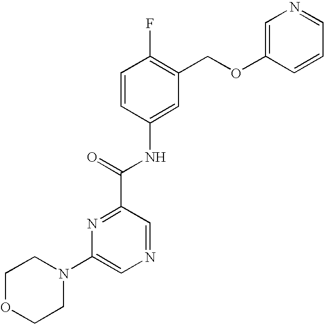 | <20 |
| 76 | 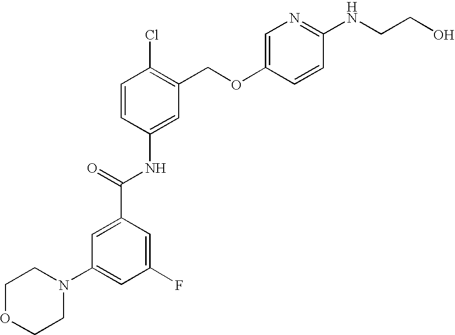 | <2 |
| 77 | 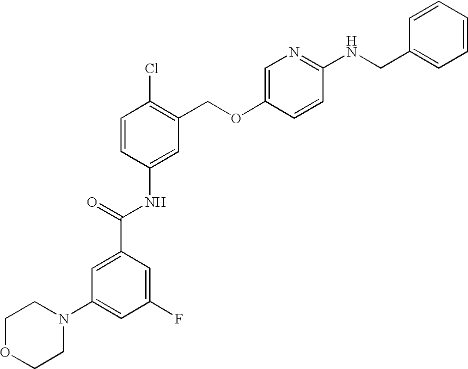 | <2 |
| 78 | 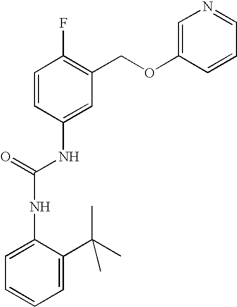 | <20 |
| 79 | 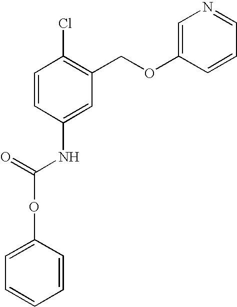 | <20 |
| 80 | 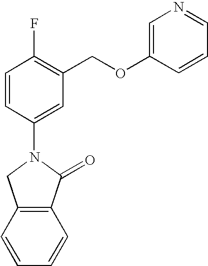 | <1000 |
| 81 | 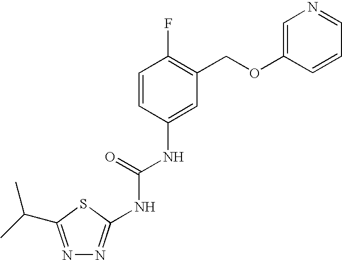 | <20 |

TABLE 2

| Compound | Structure | IC₅₀ (μM) |
|---|---|---|
| 82 | 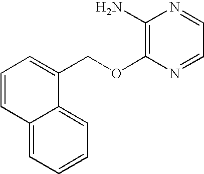 | <1000 |
| 83 | 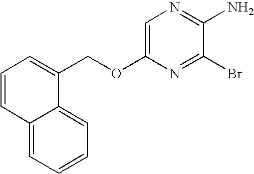 | <1000 |
| 84 | 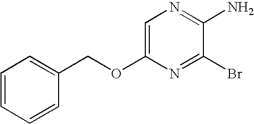 | <1000 |
| 85 | 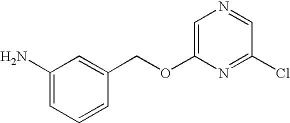 | <1000 |
| 86 | 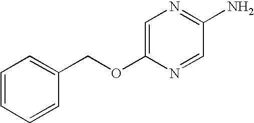 | <1000 |
| 87 | 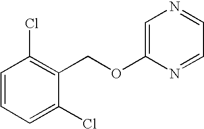 | <200 |
| 88 | 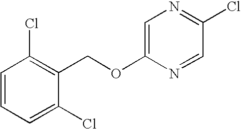 | <1000 |
| 89 | 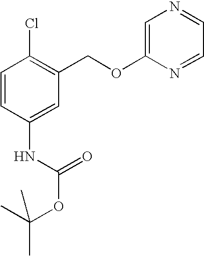 | <1000 |
| 90 | 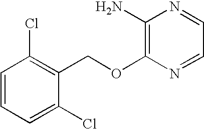 | <1000 |
| 91 | 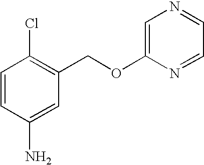 | <1000 |
| 92 | 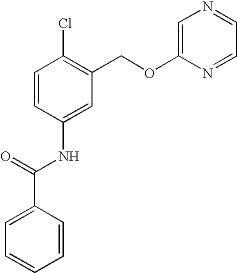 | <20 |
| 93 | 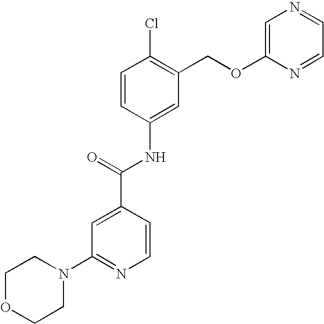 | <2 |
| 94 | 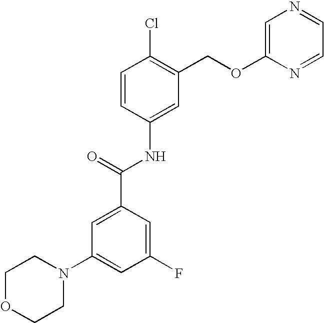 | <2 |
| 95 | 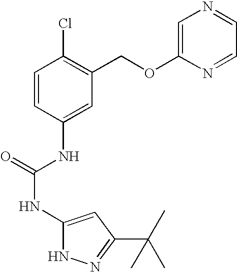 | <20 |
| 96 | 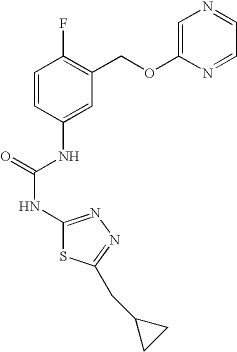 | <2 |
| 97 | 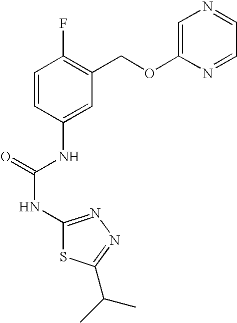 | <20 |
| 98 | 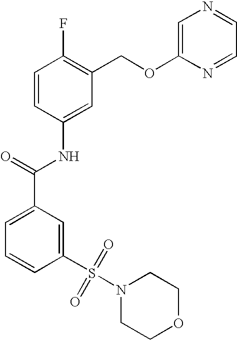 | <2 |
| 99 | 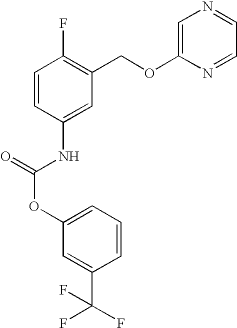 | <20 |
| 100 | 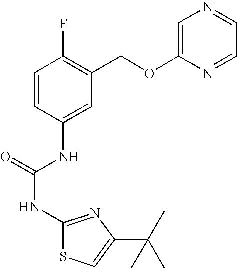 | <20 |
| 101 | 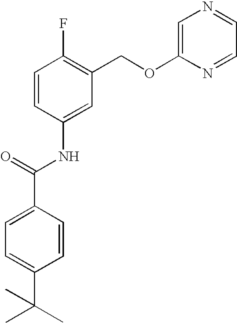 | <20 |
| 102 | 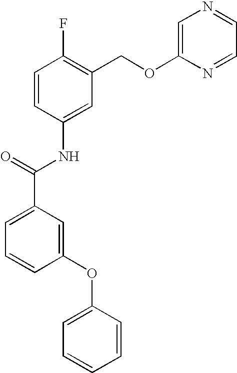 | <2 |
| 103 | 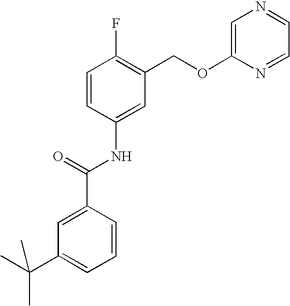 | <2 |
| 104 | 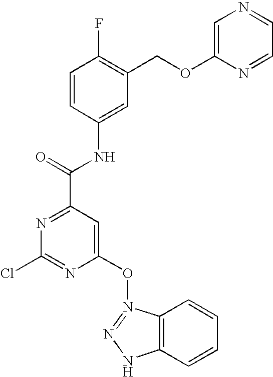 | <1000 |
| 105 | 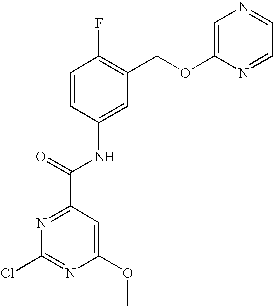 | <1000 |
| 106 | 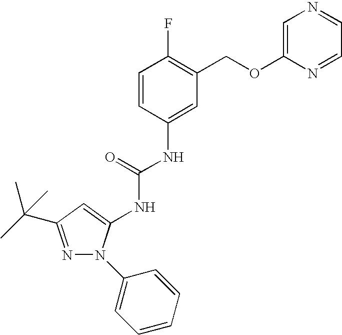 | <2 |
| 107 | 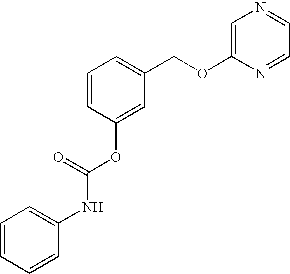 | <200 |
| 108 | 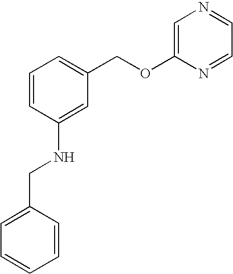 | <1000 |
| 109 | 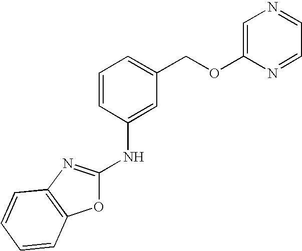 | <200 |
| 110 | 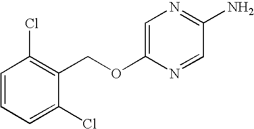 | <200 |
| 111 | 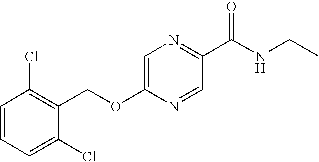 | <1000 |
| 112 | 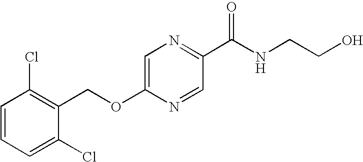 | <1000 |
| 113 | 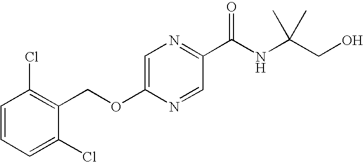 | <20 |
| 114 | 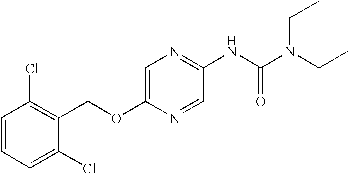 | <200 |
| 115 | 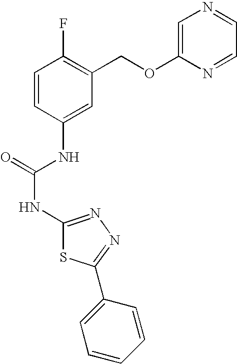 | <200 |
| 116 | 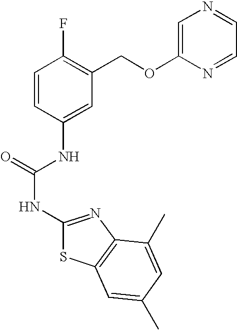 | <1000 |
| 117 | 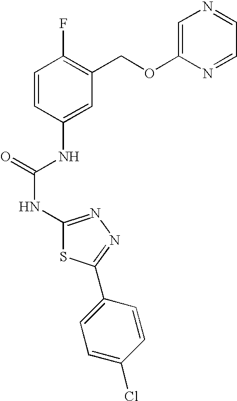 | <200 |
| 118 | 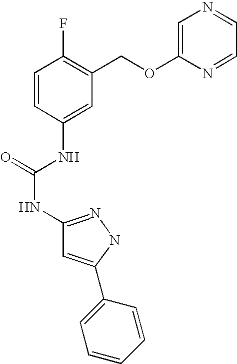 | <2 |
| 119 | 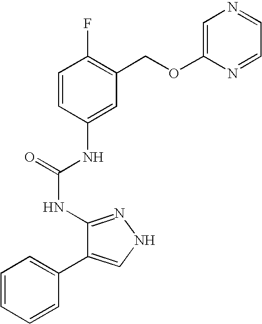 | <20 |
| 120 | 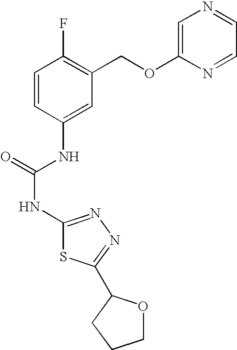 | <2 |
| 121 | 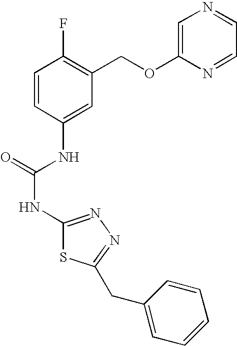 | <2 |
| 122 | 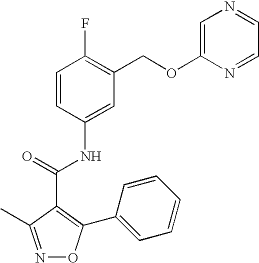 | <200 |
| 123 | 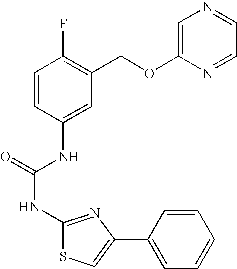 | <20 |
| 124 | 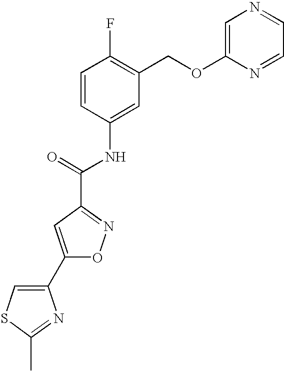 | <200 |
| 125 | 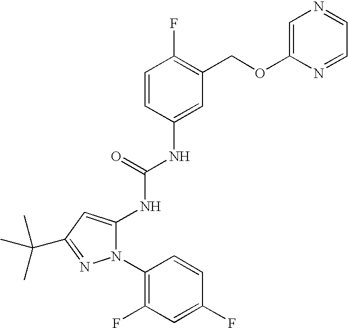 | <2 |
| 126 | 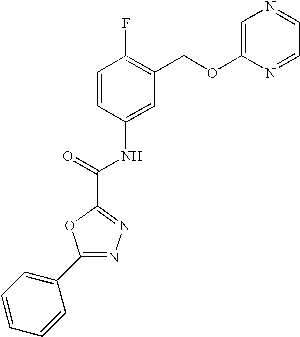 | <2 |
| 127 | 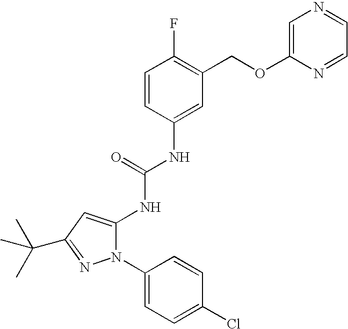 | <2 |
| 128 | 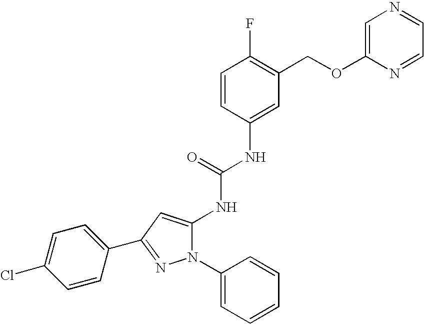 | <2 |
| 129 | 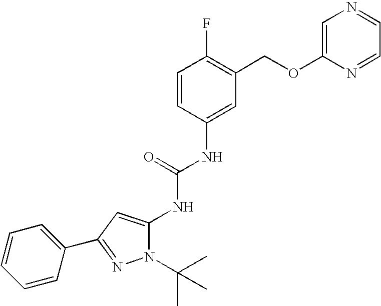 | <20 |
| 130 | 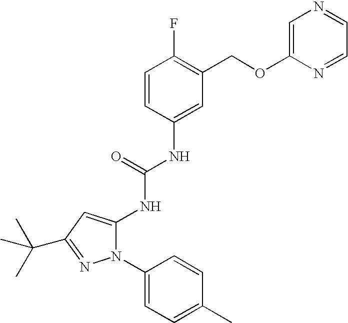 | <2 |
| 131 | 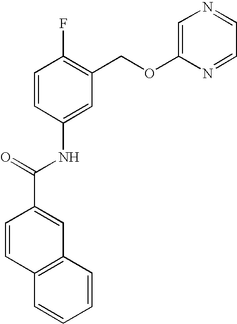 | <20 |
| 132 | 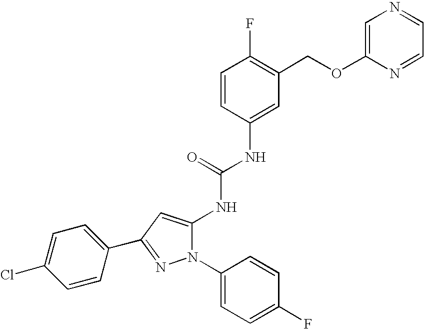 | <2 |
| 133 | 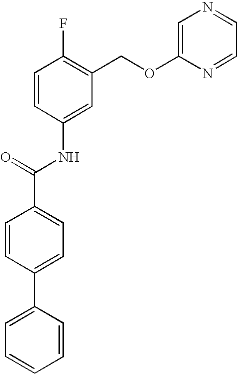 | <200 |
| 134 | 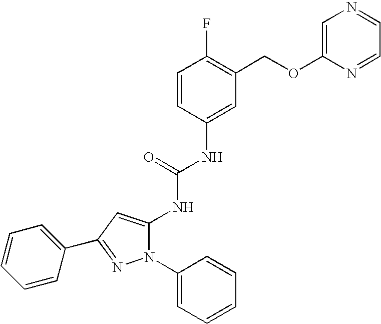 | <2 |
| 135 | 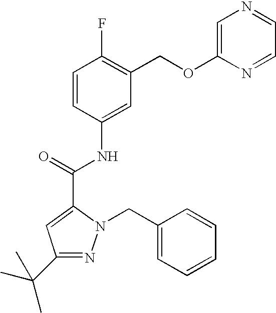 | <20 |
| 136 | 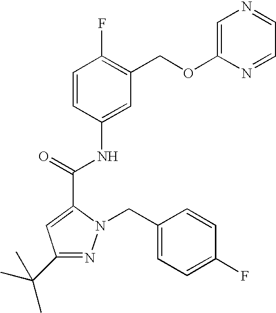 | <20 |
| 137 | 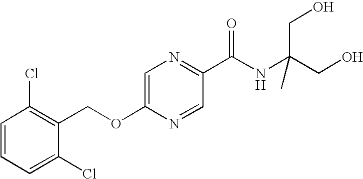 | <2 |
| 138 | 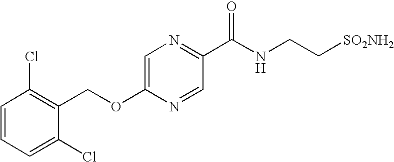 | <1000 |
| 139 | 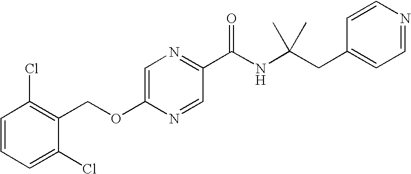 | <20 |
| 140 | 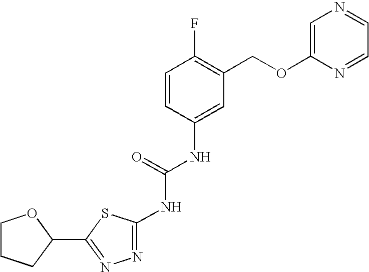 | <2 |
| 141 | 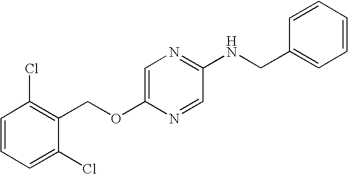 | <20 |
| 142 | 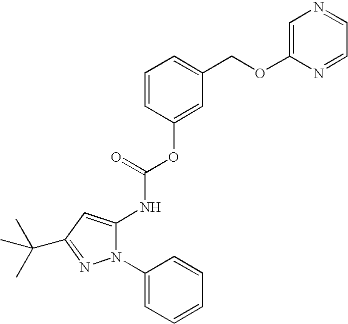 | <200 |
| 143 | 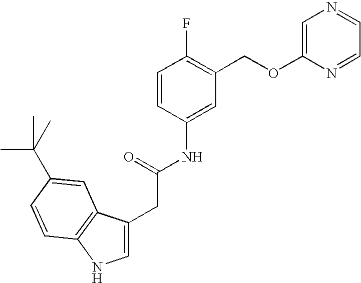 | <200 |
| 144 | 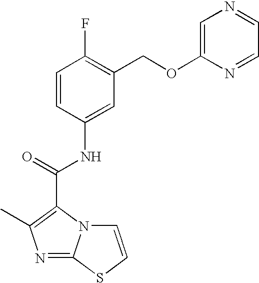 | <2 |
| 145 | 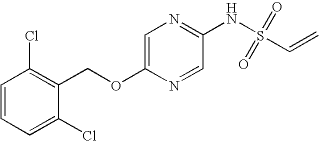 | <200 |
| 146 | 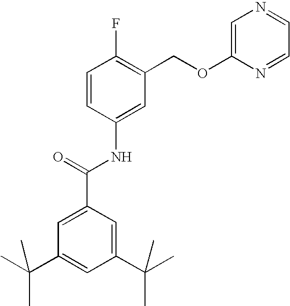 | <2 |
| 147 | 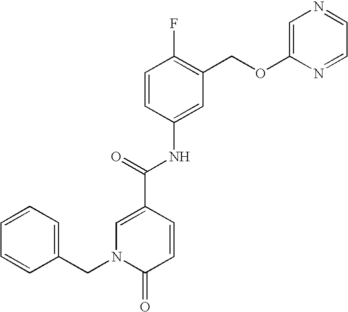 | <20 |
| 148 | 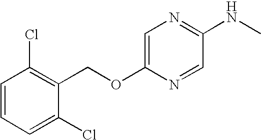 | <200 |
| 149 | 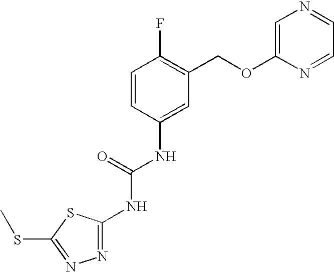 | <200 |
| 150 | 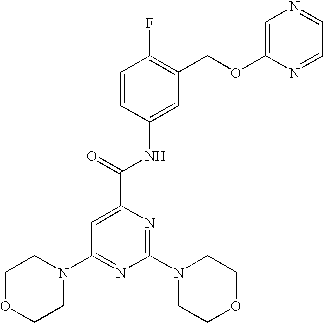 | <2 |
| 151 | 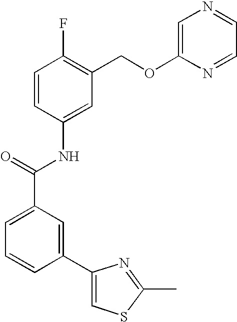 | <2 |
| 152 | 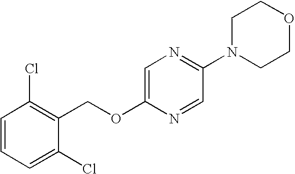 | <200 |
| 153 | 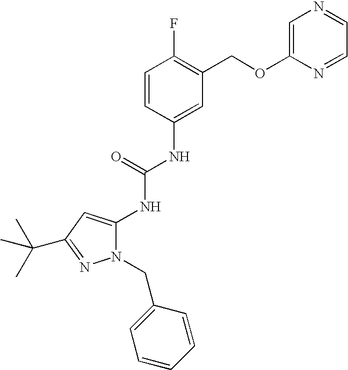 | <2 |
| 154 | 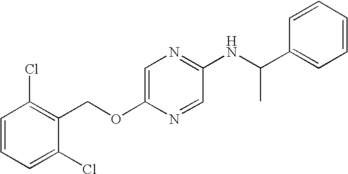 | <20 |
| 155 | 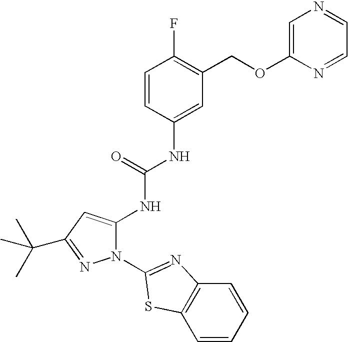 | <20 |
| 156 | 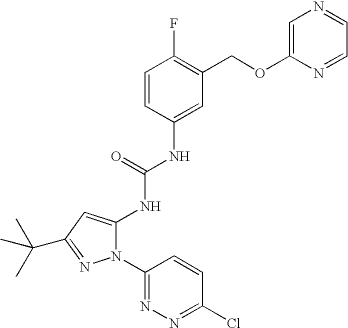 | <2 |
| 157 | 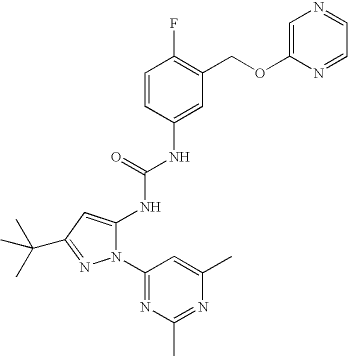 | <2 |
| 158 | 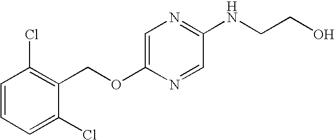 | <200 |
| 159 | 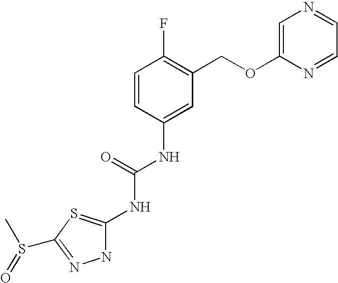 | <1000 |
| 160 | 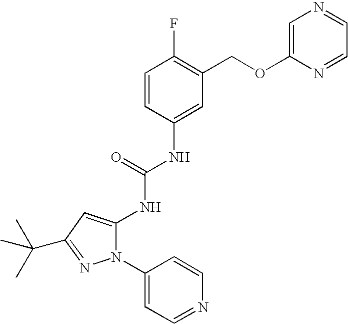 | <2 |
| 161 | 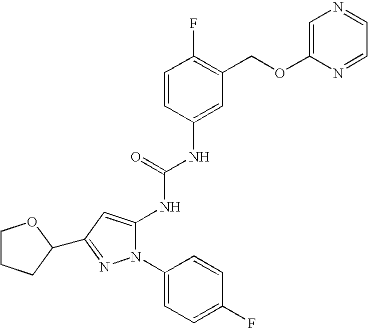 | <2 |
| 162 | 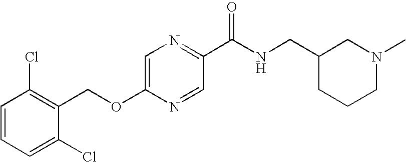 | <1000 |
| 163 | 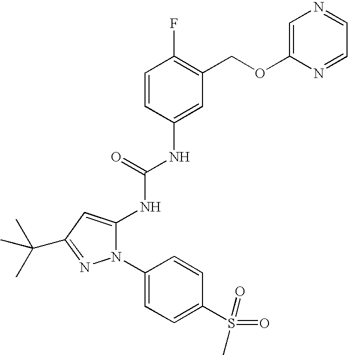 | <2 |
| 164 | 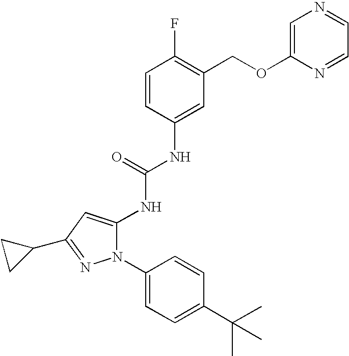 | <200 |
| 165 | 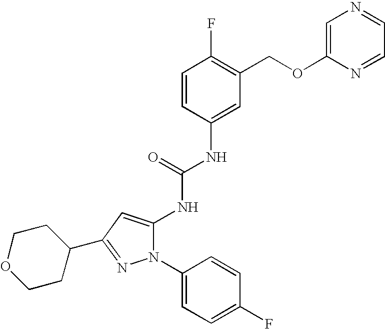 | <2 |
| 166 | 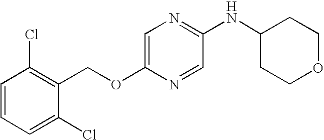 | <200 |
| 167 | 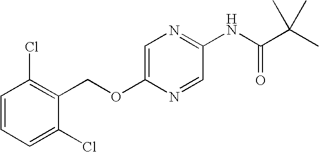 | <200 |
| 168 | 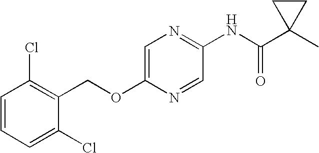 | <200 |
| 169 | 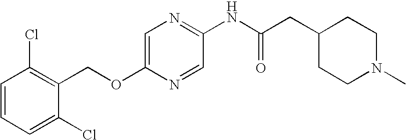 | <200 |
| 170 | 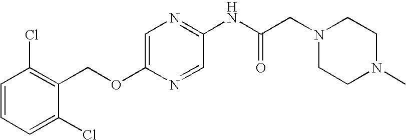 | <20 |
| 171 | 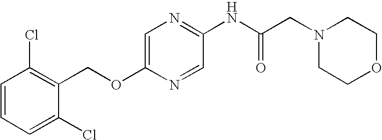 | <200 |
| 172 | 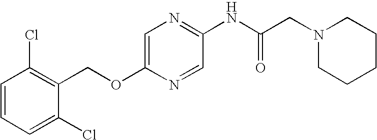 | <200 |
| 173 | 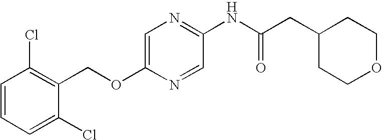 | <200 |
| 174 | 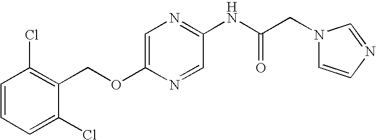 | <20 |
| 175 | 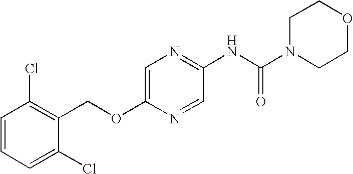 | <2 |
| 176 | 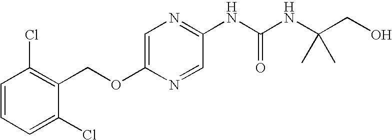 | <2 |
| 177 | 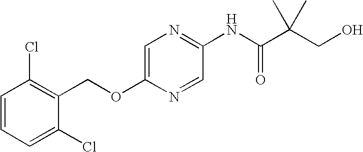 | <200 |

Inhibition of LPS-Induced TNF-α Production in THP-1 Cells The ability of the compounds of this invention to inhibit the TNF-α release was determined using a minor modification of the methods described in Rawlins P., et al., “Inhibition of endotoxin-induced TNF-α production in macrophages by 5Z-7-oxo-zeaenol and other fungal resorcyclic acid lactones,” International J. of Immunopharmacology, 21, 799, (1999).

THP-1 cells, human monocytic leukaemic cell line, ECACC) were maintained in culture medium [RPMI 1640 (Invitrogen) and 2 mM L-Glutamine supplemented with 10% foetal bovine serum (Invitrogen)] at approximately 37° C. in humidified 5% CO₂ in stationary culture.

THP-1 cells were suspended in culture medium containing 50 ng/ml PMA (SIGMA), seeded into a 96-well tissue culture plate (IWAKI) at 1×10⁵ cells/well (100 μl/well) and incubated as described above for approximately 48 hours. The medium was then aspirated, the wells washed twice in Phosphate Buffered Saline and 1 μg/ml LPS (SIGMA) in culture medium was added (200 μl/well).

Test compounds were reconstituted in DMSO (SIGMA) and then diluted with the culture medium such that the final DMSO concentration was 0.1%. Twenty microlitre aliquots of test solution or medium only with DMSO (solvent control) were added to triplicate wells immediately following LPS addition, and incubated for 6 hours as described above. Culture supernatants were collected and the amount of human TNF-α present was determined by ELISA (R&D Systems) performed according to the manufacturer's instructions.

The IC₅₀ was defined as the concentration of the test compound corresponding to half maximal inhibition of the control activity by non-linear regression analysis of their inhibition curves.

The IC₅₀ values for Compound 49, Compound 76 and Compound 94 were found to be 170 nm, 970 nM and 210 nM, respectively.

1. A compound of the formula I: 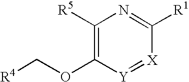
wherein:
—X═Y— is selected from —CR²═CR³— and —CR²═N—;
R¹ is selected from H, halo, NRR′, NHC(═O)R, NHC(═O)NRR′, NH₂SO₂R, and C(═O)NRR′, where R and R′ are independently selected from H and C₁₋₄ alkyl, and are optionally substituted by OH, NH₂, SQ-NH₂, C₅₋₂₀ carboaryl, C₅₋₂₀ heteroaryl and C₃₋₂₀ heterocyclyl, or may together form, with the nitrogen atom to which they are attached, an optionally substituted nitrogen containing C₅₋₇ heterocyclyl group;
R² and R³ (where present) are independently selected from H, optionally substituted C₁₋₇ alkyl, optionally substituted C₅₋₂₀ aryl, optionally substituted C₃₋₂₀ heterocyclyl, halo, amino, amido, hydroxy, ether, thio, thioether, acylamido, ureido and sulfonamino;
R⁴ an optionally substituted C₅₋₂₀ carboaryl or C₅₋₂₀ heteroaryl group; and
R⁵ is selected from R⁵′, halo, NHR^{5′}, C(═O)NHR⁵′, OR^{5′}, SR^{5′}, NHC(═O)R⁵′, NHC(═O)NHR^{5′}, NHS(═O)₂R⁵′, wherein R⁵′ is H or C₁₋₃ alkyl (optionally substituted by halo, NH₂, OH, SH);
and pharmaceutically acceptable salts thereof for use in a method of therapy.

2. A compound according to claim 1, wherein —X═Y— is —CR²═N—.

3. A compound according to claim 1, wherein R⁵ is selected from R^{5′}, halo, NHR^{5′}, OR^{5′}, SR⁵′, wherein R^{5′} is H or C₁₋₃ alkyl, optionally substituted by halo, NH₂, OH, SH.

4. A compound according to claim 3, wherein R⁵ is selected from H and NH₂.

5. A compound according to claim 1, wherein R¹ is selected from H, NRR′, NHC(═O)R, NHC(═O)NRR′, and NH₂SO₂R.

6. A compound according to claim 5, wherein R1 is selected from H and NH₂.

7. A compound according to claim 1, wherein R² and R³ (where present) are independently selected from H, halo, amino, hydroxy and thio.

8. A compound according to claim 7, wherein R² and R³ (where present) are selected from H and halo.

9. A compound according to claim 1, wherein R4 is an optionally substituted C₅₋₁₀ aryl group.

10. A compound according to claim 9, wherein R⁴ is selected from a C₅₋₁₀ carboaryl group and a C₅₋₁₀ heteroaryl group having one or two nitrogen ring atoms.

11. A compound according to claim 10, wherein R⁴ is an optionally substituted phenyl or napthyl group.

12. A compound according to claim 11, wherein R⁴ is a phenyl group substituted with one or two substituents independently selected from halo, ether, C₁₋₇ alkyl, C₅₋₂₀ aryl, amido, acylamido, ureido, carbamate and reverse carbamate.

13. A compound according to claim 1 of either formula IIa formula IIb: 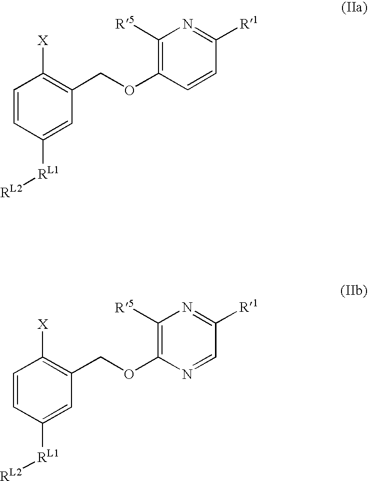
wherein:
R′¹ is selected from H, NR^{C1}R^{C2}, NRC(═O)RC′, NHC(═O)NR^{C1}R^{C2}, NH₂SO₂K^{C1}, and C(═O)NR^{C1}R^{C2}, where R^{C1} and R^{C2} are independently selected from H and C₁₋₄ alkyl, and are optionally substituted by OH, NH₂, C₅₋₂₀ carboaryl, and C₅₋₂₀ heteroaryl, or may together form, with the nitrogen atom to which they are attached, an optionally substituted nitrogen containing C₅₋₇ heterocyclyl group;
R′⁵ is selected from H and NH₂;
X is selected from H and halo;
R^{L1} is selected from —NH—C(═O)—, —NH—C(═O)—NH—, —NH—C(═O)—O— or p1 —O—C(═O)—NH—;
R^{L2} is selected from H, optionally substituted C₅₋₂₀ carboaryl and optionally substituted C₅₋₂₀ heteroaryl, except that R^{L2} cannot be H when R^{L1} is —NH—C(═O)—O—.

14. A compound according to claim 13 of formula IIa.

15. A compound according to claim 14, wherein
R′¹ is selected from H and NR^{C1}R^{C2}.

16. A compound according to claim 15, wherein R′¹ is selected from H and NHR^{C1}.

17. A compound according to claim 14, wherein R′⁵ is H.

18. A compound according to claim 14, wherein X is halo.

19. A compound according to claim 14, wherein R^{L1} is —NH—C(═O)—.

20. A compound according to claim 14, wherein R^{L2} is a C₅₋₂₀ carboaryl or C₅₋₂₀ heteroaryl group.

21. A compound according to claim 13, of formula IIb.

22. A compound according to claim 21, wherein R′¹ is selected from H and NR^{C1}R^{C2}.

23. A compound according to claim 21, wherein R′5 is H.

24. A compound according to claim 21, wherein X is halo.

25. A compound according to claim 21, wherein R^{L1} is —NH—C(═O)—NH—.

26. A compound according to claim 21, wherein R^{L2} is a C₅₋₂₀ carboaryl or C₅₋₂₀ heteroaryl group.

27. A compound of formula IIa or IIb as described in claim 13, or an isomer, salt, solvate or prodrugs thereof.

28. A composition comprising a compound according to claim 1 and a pharmaceutically acceptable carrier or diluent.

29. The use of a compound according to claim 1 for the manufacture of a medicament for use in the treatment of condition ameliorated by the inhibition of p38 MAP kinase.

30. The use according to claim 29, wherein the conditions ameliorated by the inhibition of p38 MAP kinase is an arthritic condition.

31. A method for the treatment of a condition ameliorated by the inhibition of p38 MAP kinase comprising administering to a subject suffering from said a condition ameliorated by the inhibition of p38 MAP kinase a therapeutically-effective amount of a compound according to claim 1.

32. The method according to claim 29, wherein the conditions ameliorated by the inhibition of p38 MAP kinase is an arthritic condition.
